(19) United States
(12) Patent Application Publication
(10) Pub. No.: US 2023/0277698 A1
(43) Pub. Date: Sep. 7, 2023

(54) SILICON-CONTAINING LIGAND COMPOUNDS

(71) Applicant: Technische Universität München, München (DE)

(72) Inventors: Alexander Wurzer, Landshut (DE); Hans-Jürgen Wester, Schweitenkirchen (DE); Sebastian Fischer, Dachau (DE); Jan-Philip Kunert, Augsburg (DE)

(21) Appl. No.: 18/006,072

(22) PCT Filed: Jul. 23, 2021
(86) PCT No.: PCT/EP2021/070686

§ 371 Date: Jan. 19, 2023

(30) Foreign Application Priority Data
Jul. 23, 2020 (EP) 20187504.4

(51) Int. Cl. A61K 51/04; A61P 35/00

(52) U.S. Cl. CPC A61K 51/0497; A61P 35/00

(57) ABSTRACT

Provided is a ligand compound, comprising (a) a targeting group, (b) one or more chelating groups, optionally containing a chelated radioactive or non-radioactive cation, and (c) a group carrying an Si—OH functional moiety. The ligand compound is suitable for use in therapeutic or diagnostic methods, especially in radiotherapy or radiodiagnosis of cancer, such as prostate cancer.

The present invention relates to silicon-containing ligand compounds, comprising, within in a single molecule: (a) a targeting group, (b) one or more chelating groups, optionally containing a chelated nonradioactive or radioactive cation and (c) a group carrying an Si—OH functional moiety.

### Cancer, in Particular Prostate Cancer

In 2017, 9.6 million people are estimated to have died from the various forms of cancer. Every sixth death in the world is due to cancer, making it the second leading cause of death; second only to cardiovascular diseases.

Prostate Cancer (PCa) remained over the last decades the most common malignant disease in men with high incidence for poor survival rates. Due to its overexpression in prostate cancer (Silver et al., Clinical Cancer Research 3, 81-85 (1997)), prostate-specific membrane antigen (PSMA) or glutamate carboxypeptidase II (GCP II) proved its eligibility as excellent target for the development of highly sensitive radiolabelled agents for endoradiotherapy and imaging of PCa (Afshar-Oromieh et al., European journal of nuclear medicine and molecular imaging 42, 197-209 (2015); Benešová et al., Journal of Nuclear Medicine 56, 914-920 (2015); Robu et al., Journal of Nuclear Medicine, jnumed. 116.178939 (2016); Weineisen et al.; Journal of Nuclear Medicine 55, 1083-1083 (2014); Rowe et al., Prostate cancer and prostatic diseases (2016); Maurer et al., Nature Reviews Urology (2016)). Prostate-specific membrane antigen is an extracellular hydrolase whose catalytic center comprises two zinc(II) ions with a bridging hydroxido ligand. It is highly upregulated in metastatic and hormone-refractory prostate carcinomas, but its physiologic expression has also been reported in kidneys, salivary glands, small intestine, brain and, to a low extent, also in healthy prostate tissue. In the intestine, PSMA facilitates absorption of folate by conversion of pteroylpoly-γ-glutamate to pteroylglutamate (folate). In the brain, it hydrolyses N-acetyl-Laspartyl-L-glutamate (NAAG) to N-acetyl-L-aspartate and glutamate.

Prostate cancer is not the only cancer to express PSMA. Nonprostate cancers to demonstrate PSMA expression include breast, lung, colorectal, and renal cell carcinoma.

### Tumor Antigens, in Particular Prostate-Specific Membrane Antigen (PSMA)

In view of the high incidence of cancer mortality there is an ongoing need for the development of novel means and methods for treating and diagnosing tumors. The means and methods for treating and diagnosing tumors can be made specific for tumor cells by specifically targeting tumor antigens. A tumor-specific antigen is a protein or other molecule that is found only or preferentially found on tumor cells and not or significantly less on normal cells. Tumor antigens can help the body to develop an immune response against cancer cells. They may therefore be used as possible targets for a targeted therapy or for an immunotherapy to help boost the body's immune system to kill or weaken the tumor cells. Tumor-specific antigens may also be used in laboratory tests to help diagnose some types of cancer. An example of such a tumor antigen is PSMA.

The general necessary structures of PSMA targeting molecules comprise a binding unit that encompasses a zinc-binding group (such as urea (Zhou et al., Nature Reviews Drug Discovery 4, 1015-1026 (2005)), phosphinate or phosphoramidate) connected to a P1′ glutamate moiety, which warrants high affinity and specificity to PSMA and is typically further connected to an effector functionality (Machulkin et al., Journal of drug targeting, 1-15 (2016)). The effector part is more flexible and to some extent tolerant towards structural modifications. The entrance tunnel accommodates two other prominent structural features, which are important for ligand binding. The first one is an arginine patch, a positively charged area at the wall of the entrance funnel and the mechanistic explanation for the preference of negatively charged functionalities at the P1 position of PSMA. This appears to be the reason for the preferable incorporation of negative charged residues within the ligand-scaffold. An in-depth analysis about the effect of positive charges on PSMA ligands has been, to our knowledge, so far not conducted. Upon binding, the concerted repositioning of the arginine side chains can lead to the opening of an S1 hydrophobic accessory pocket, the second important structure that has been shown to accommodate an iodo-benzyl group of several urea based inhibitors, thus contributing to their high affinity for PSMA (Barinka et al., Journal of medicinal chemistry 51, 7737-7743 (2008)).

Zhang et al. discovered a remote binding site of PSMA, which can be employed for bidentate binding mode (Zhang et al., Journal of the American Chemical Society 132, 12711-12716 (2010)). The so called arene-binding site is a simple structural motif shaped by the side chains of Arg463, Arg511 and Trp541, and is part of the GCPII entrance lid. The engagement of the arene binding site by a distal inhibitor moiety can result in a substantial increase in the inhibitor affinity for PSMA due to avidity effects. PSMA I&T was developed with the intention to interact this way with PSMA, albeit no crystal structure analysis of binding mode is available. A necessary feature according to Zhang et al. is a linker unit (Suberic acid in the case of PSMA I&T) which facilitates an open conformation of the entrance lid of GCPII and thereby enabling the accessibility of the arene-binding site. It was further shown that the structural composition of the linker has a significant impact on the tumor-targeting and biologic activity as well as on imaging contrast and pharmacokinetics (Liu et al., Bioorganic & medicinal chemistry letters 21, 7013-7016 (2011)), properties which are crucial for both high imaging quality and efficient targeted endoradiotherapy.

Two categories of PSMA targeting inhibitors are currently used in clinical settings. On the one side there are tracers with chelating units for radionuclide complexation such as PSMA I&T or related compounds (Kiess et al., The quarterly journal of nuclear medicine and molecular imaging 59, 241 (2015)). On the other side there are small molecules, comprising a targeting unit and effector molecules.

The most often used agents for selective PSMA imaging are PSMA HBED-CC (Eder et al., Bioconjugate chemistry 23, 688-697 (2012)), PSMA-617 (Benešová et al., Journal of Nuclear Medicine 56, 914-920 (2015)) and PSMA I&T (Weineisen et al.; Journal of Nuclear Medicine 55, 1083-1083 (2014)), which are predominantly labelled with ⁶⁸Ga (88.9% β*, E_{β+,max}=1.89 MeV, t_{1/2}=68 min). Among these ⁶⁸Ga-PSMA-HBED-CC (also known as ⁶⁸Ga-PSMA-11), is so far considered as the golden standard for PET imaging of PCa.

WO 2019/020831 A1 discloses conjugate compounds comprising a ligand (which may be a PSMA ligand) in combination with a silicon-fluoride acceptor (SiFA) moiety and one or more chelating groups. The conjugate compounds can be used as dual-mode radiotracer or radiotherapeutics. They are also referred to as radiohybrid. (rh) compounds.

In view of the above, the technical problem underlying the present invention can be seen in providing compounds which can be used as radiodiagnostics or radiotherapeutics, or as precursors thereof, in particular in diagnosis or treatment of prostate cancer, and which are characterized by favourable pharmacological and pharmacokinetic properties.

This technical problem is solved by the subject-matter of the claims.

In accordance with a main aspect thereof, the invention thus provides a ligand compound, comprising (a) a targeting group, such as a PSMA binding group, (b) one or more chelating groups optionally containing a chelated radioactive or non-radioactive cation and (c) a group carrying an Si—OH functional moiety.

It has been found by the present inventors that the compounds in accordance with the present invention are suitable as radiopharmaceuticals, in particular radiodiagnostics, radiotherapeutics, or precursors thereof without the need for a SiFA moiety, while maintaining favourable pharmacological and pharmacokinetic properties. In particular, the compounds in accordance with the invention exhibit a favorable biodistribution. Based on a PSMA binding group as an exemplary targeting group, it is demonstrated in the examples section that the ligand compounds in accordance with the invention are particularly suitable for treating a tumor, noting that PSMA is prostate cancer specific tumor antigen.

As will be discussed in more detail herein below, the compounds in accordance with the present invention are likewise suitable as radiodiagnostics or precursors thereof, noting that characteristic structures related to a disease or disorder which are accessible for a targeting group, like tumor antigens, can be targeted for diagnosis as well as for therapy.

In this context, the inventors have found that the presence of a group carrying an Si—OH functional moiety leads to binding properties of the ligand compounds towards plasma proteins, in particular albumin, which allow the half-life of the compounds in blood plasma to be reduced compared to radiohybrid (rh) ligand compounds containing ¹⁸F bound to a silicon atom and a chelated cold (non-radioactive) metal or ¹⁹F bound to a silicon atom and a chelated radiometal. Thus, for example, an accelerated excretion of the ligand compounds, and/or a favorable dosimetry can be achieved by increasing the tumor-to-kidney ratio. Thus, for example, the presence of the Si—OH (silanol) functional groups in the compounds in accordance with the invention allows their excretion kinetics to be optimized for diverse therapeutic applications. Due to a structural relationship between the SiFA-group-containing rh compounds, the respective therapeutic use of the compounds in accordance with the invention can be suitably linked to a diagnostic step relying on such a SiFA group containing compound which is marked by an ¹⁸F atom.

In accordance with a further aspect, the present invention relates to a pharmaceutical or diagnostic composition comprising or consisting of one or more compounds in accordance with the present invention, as well as a compound in accordance with the invention for use in a method of diagnosing and/or treating cancer, preferably prostate cancer; or neoangiogenesis/angiogenesis.

Embodiments of the present invention are summarized in the following items.
1. A ligand compound, comprising:
   (a) a targeting group,
   (b) one or more chelating groups, optionally containing a chelated radioactive or non-radioactive cation, and
   (c) a group carrying an Si—OH functional moiety.
2. The ligand compound in accordance with item 1, wherein the targeting group is a PSMA binding group.
3. The ligand compound in accordance with item 1 or 2, wherein the group carrying an Si—OH functional moiety is a group of formula (S-1)

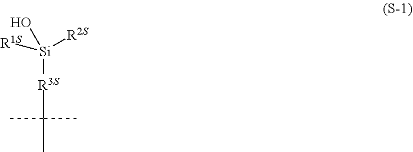

wherein
R^{1S} and R^{2S} are independently a linear or branched C3 to C10 alkyl group, preferably
R^{1S} and R^{2S} are selected from isopropyl and tert-butyl, and more preferably R^{1S} and R^{2S} are tert-butyl;
R^{3S} is a C1 to C20 hydrocarbon group which comprises one or more aromatic and/or aliphatic units, and which optionally comprises up to 3 heteroatoms independently selected from O, N and S, preferably R^{3S} is a C6 to C10 hydrocarbon group which comprises an aromatic ring and which may comprise one or more aliphatic units; more preferably R^{3S} is a phenylene group or a benzyl group carrying the Si atom shown in the formula attached as a substituent to its aromatic ring, and most preferably, R^{3S} is a phenylene group, and the Si atom and the bond marked by the dashed line are in a para-position to each other, or a benzyl group wherein the bond with the dashed line is formed by the —CH₂— moiety at the benzyl group, and the Si atom attached as a substituent to the aromatic ring and the —CH₂— moiety of the benzyl group are in a para-position to each other;
and wherein the group carrying an Si—OH functional moiety of formula (S-1) is attached to the remainder of the compound via the bond marked by the dashed line.
4. The PSMA ligand compound in accordance with item 3, wherein the group carrying an Si—OH functional moiety is a group of formula (S-2) or (S-3)

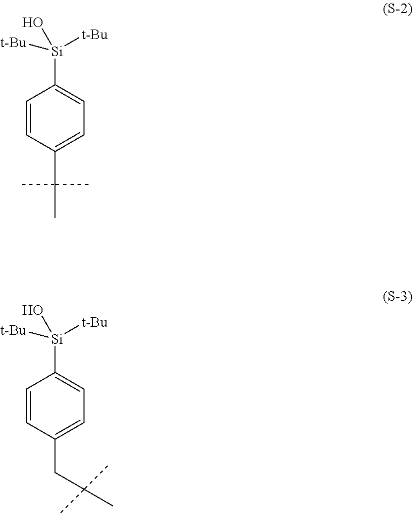

wherein t-Bu indicates a tert-butyl group,
and wherein the group carrying an Si—OH functional moiety of formula (S-2) and (S-3) is attached to the remainder of the compound via the bond marked by the dashed line.
5. The ligand compound in accordance with any of items 1 to 4, wherein the targeting group is a PSMA binding group of formula (P-1) or a pharmaceutically acceptable salt thereof

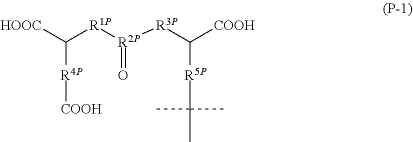

wherein:
R^{1P} is CH₂, NH or O, preferably NH;
R^{3P} is CH₂, NH or O, preferably NH;
R^{2P} is C or P(OH), preferably C;
R^{4P} is selected from
a group —(CH₂)ₘ—, wherein m is an integer of 2 to 6, preferably 2 to 4, more preferably 2, and
a group *—(CH₂)ₚ—NH—C(O)—, wherein p is an integer of 1 to 5, preferably 1 to 3, more preferably 1, and the bond marked with * faces upwards from R^{4P} in formula (P-1);
R^{5P} is selected from
a group —(CH₂)ₙ—, wherein n is an integer of 1 to 6, preferably 2 to 4, more preferably 2 or 4, and
a group *—(CH₂)_{q}—NH—C(O)—, wherein q is an integer of 1 to 5, preferably 1 to 3, more preferably 1, and the bond marked with * faces upwards from R^{5P} in formula (P-1);
and wherein the PSMA binding group is attached to the remainder of the compound via the bond marked by the dashed line.
6. The ligand compound in accordance with any of items 1 to 5, wherein the targeting group is a PSMA binding group of formula (P-2) or a pharmaceutically acceptable salt thereof

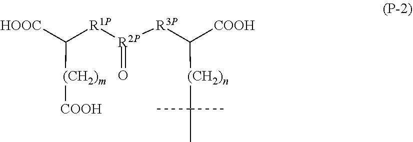

wherein:
m is an integer of 2 to 6, preferably 2 to 4, more preferably 2;
n is an integer of 1 to 6, preferably 2 to 4, more preferably 2 or 4;
R^{1P} is CH₂, NH or O, preferably NH;
R^{3P} is CH₂, NH or O, preferably NH;
R^{2P} is C or P(OH), preferably C;
and wherein the PSMA binding group is attached to the remainder of the compound via the bond marked by the dashed line.
7. The ligand compound in accordance with item 5 or 6, wherein R^{1P} is NH, R^{3P} is NH, and R^{2P} is C.
8. The ligand compound in accordance with any of items 1 to 7, wherein the targeting group is a PSMA binding group of formula (P-3), or a pharmaceutically acceptable salt thereof:

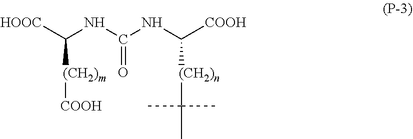

wherein:
m is an integer of 2 to 6, preferably 2 to 4, more preferably 2;
n is an integer of 1 to 6, preferably 2 to 4, more preferably 2 or 4;
and wherein the PSMA binding group is attached to the remainder of the compound via the bond marked by the dashed line.
9. The ligand compound in accordance with any of items 5 to 8, wherein m is 2.
10. The ligand compound in accordance with any of items 5 to 9, wherein n is 2 or 4.
11. The ligand compound in accordance with any of items 1 to 10, wherein the chelating group comprises at least one of
   (i) a macrocyclic ring structure with 8 to 20 ring atoms of which 2 or more, preferably 3 or more, are selected from oxygen atoms and nitrogen atoms; and
   (ii) an acyclic, open chain chelating structure with 8 to 20 main chain atoms of which 2 or more, preferably 3 or more are heteroatoms selected from oxygen atoms and nitrogen atoms.
12. The ligand compound in accordance with any of items 1 to 11, wherein the chelating group is a residue of a chelating agent selected from bis(carboxymethyl)-1,4,8,11-tetraazabicyclo[6.6.2]hexadecane (CBTE2a), cyclohexyl-1,2-diaminetetraacetic acid (CDTA), 4-(1,4,8,11-tetraazacyclotetradec-1-yl)-methylbenzoic acid (CPTA), N′-[5-[acetyl(hydroxy)amino]pentyl]-N-[5-[[4-[5-aminopentyl-(hydroxy)amino]-4-oxobutanoyl]amino]pentyl]-N-hydroxybutandiamide (DFO), 4,11-bis(carboxymethyl)-1,4,8,11-tetraazabicycle[6.6.2]hexadecan (DO2A) 1,4,7,10-tetraazacyclododecan-N,N′,N″,N″′-tetraacetic acid (DOTA), 2-[1,4,7,10-tetraazacyclododecane-4,7,10-triacetic acid]-pentanedioic acid (DOTAGA or DOTA-GA), N,N′-dipyridoxylethylendiamine-N,N′-diacetate-5,5′-bis(phosphat) (DPDP), diethylenetriaminepentaacetic acid (DTPA), ethylenediamine-N,N′-tetraacetic acid (EDTA), ethyleneglykol-O,O-bis(2-aminoethyl)-N,N,N′,N′-tetraacetic acid (EGTA), N,N-bis(hydroxybenzyl)-ethylenediamine-N,N′-diacetic acid (HBED), hydroxyethyldiaminetriacetic acid (HEDTA), 1-(p-nitrobenzyl)-1,4,7,10-tetraazacyclo-decan-4,7,10-triacetate (HP-DOA3), 6-hydrazinyl-N-methylpyridine-3-carboxamide (HYNIC), 1,4,7-triazacyclononan-1-succinic acid-4,7-diacetic acid (NODASA), 1-(1-carboxy-3-carboxypropyl)-4,7-(carboxy)-1,4,7-triazacyclononane (NODAGA), 1,4,7-triazacyclononanetriacetic acid (NOTA), 4,11-bis(carboxymethyl)-1,4,8,11-tetraaza-bicyclo[6.6.2]hexadecane (TE2A), 1,4,8,11-tetraazacyclododecane-1,4,8,11-tetra-acetic acid (TETA), terpyridine-bis(methyleneamine) tetraacetic acid (TMT), 1,4,7,10-tetraazacyclotridecan-N,N′,N″,N″′-tetraacetic acid (TRITA), and triethylenetetra-aminehexaacetic acid (TTHA), N,N′-bis[(6-carboxy-2-pyridil)methyl]-4,13-diaza-18-crown-6 (H₂macropa), 4-amino-4-{2-[(3-hydroxy-1,6-dimethyl-4-oxo-1,4-dihydro-pyridin-2-ylmethyl)-carbamoyl]-ethyl} heptanedioic acid bis-[(3-hydroxy-1,6-dimethyl-4-oxo-1,4-dihydro-pyridin-2-ylmethyl)-amide] (THP), 1,4,7-triazacyclononane-1,4,7-tris[methylene(2-carboxyethyl)phosphinic acid (TRAP), 2-(4,7,10-tris(2-amino-2-oxoethyl)-1,4,7,10-tetraazacyclododecan-1-yl)acetic acid (DO3AM), and 1,4,7,10-tetraazacyclododecane-1,4,7,10-tetrakis[methylene(2-carboxyethylphosphinic acid)](DOTPI), S-2-(4-isothiocyanatobenzyl)-1,4,7,10-tetraazacyclododecane tetraacetic acid, and from pharmaceutically acceptable salts thereof,
   which residue is bound to the remainder of the compound via an ester bond, an amide bond, or a thiourea bond, preferably an amide bond, said bond being formed using a functional group contained in the chelating agent,
   and wherein the chelating group optionally contains a chelated radioactive or non-radioactive cation.
13. The ligand compound of item 11, wherein the chelating agent contains a carboxy group, and the residue of the chelating agent is bound to the remainder of the compound via an amide bond formed using the carboxy group.
14. The ligand compound of any of items 1 to 13, wherein the chelating group is selected from a group of the formula (CH-1) or (CH-2), or a pharmaceutically acceptable salt thereof

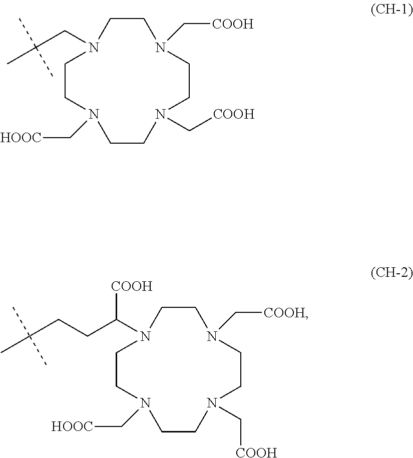

which chelating group is attached by the bond marked by the dashed line to the remainder of the compound via an ester or an amide bond, preferably an amide bond, and wherein the chelating group optionally contains a chelated radioactive or non-radioactive cation.
15. The ligand compound in accordance with any of items 1 to 14, wherein the chelating group contains a chelated cation selected from the cations of ⁴³Sc, ⁴⁴Sc, ⁴⁷Sc, ⁵¹Cr, ^{52m}Mn, ⁵⁸Co, ⁵²Fe, ⁵⁶Ni, ⁵⁷Ni, ⁶²Cu, ⁶⁴Cu, ⁶⁷Cu, ⁶⁶Ga, ⁶⁸Ga, ⁶⁷Ga, ⁸⁹Zr, ⁹⁰Y, ⁸⁶Y, ⁹⁴mTc, ^{99m}Tc, ⁹⁷Ru, ¹⁰⁵Rh, ¹⁰⁹Pd, ¹¹¹Ag, ^{110m}In, ¹¹¹In, ^{113m}In, ^{114m}In, ^{117m}Sn, ¹²¹Sn, ¹²⁷Te, ¹⁴²Pr, ¹⁴³Pr, ¹⁴⁷Nd, ¹⁴⁹Gd, ¹⁴⁹Pm, ¹⁵¹Pm, ¹⁴⁹Tb, ¹⁵²Tb, ¹⁵⁵Tb, ¹⁵³Sm, ¹⁵⁶Eu, ¹⁵⁷Gd, ¹⁶¹Tb, ¹⁶⁴Tb, ¹⁶¹Ho, ¹⁶⁶Ho, ¹⁵⁷Dy, ¹⁶⁵Dy, ¹⁶⁶Dy, ¹⁶⁰Er, ¹⁶⁵Er, ¹⁶⁹Er, ¹⁷¹Er, ¹⁶⁶Yb, ¹⁶⁹Yb, ¹⁷⁵Yb, ¹⁶⁷Tm, ¹⁷²Tm, ^{nat}Lu, ¹⁷⁷Lu, ¹⁸⁶Re, ¹⁸⁸Re, ¹⁸⁸W, ¹⁹¹Pt, ^{195m}Pt, ¹⁹⁴Ir, ¹⁹⁷Hg, ¹⁹⁸Au, ¹⁹⁹Au, ²¹²Pb, ²⁰³Pb, ²¹¹At, ²¹²Bi, ²¹³Bi, ²²³Ra, ²²⁴Ra, ²²⁵Ac, and ²²⁷Th, or a cationic molecule comprising ¹⁸F, such as ¹⁸F-[AIF]²⁺.
16. The ligand compound in accordance with any of items 1 to 15, wherein the chelating group contains a chelated Ga cation or a chelated Lu cation.
17. The ligand compound in accordance with any of items 1 to 16, wherein the chelating group contains a chelated radioactive cation.
18. The ligand compound in accordance with item 16 or 17, wherein the chelating group contains a chelated ¹⁷⁷Lu cation.
19. The ligand compound in accordance with item 1, which is a PSMA ligand compound of formula (I) or a pharmaceutically acceptable salt thereof

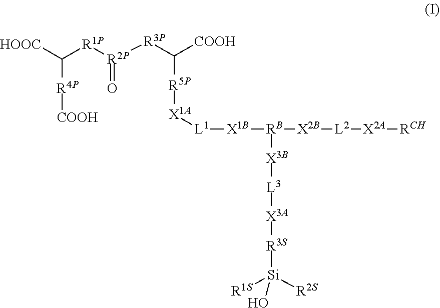

wherein, in formula (I),
   R^{1P} is CH₂, NH or O, preferably NH;
   R^{3P} is CH₂, NH or O, preferably NH;
   R^{2P} is C or P(OH), preferably C;
   R^{4P} is selected from
a group —(CH₂)ₘ—, wherein m is an integer of 2 to 6, preferably 2 to 4, more preferably 2, and
a group *—(CH₂)ₚ—NH—C(O)—, wherein p is an integer of 1 to 5, preferably 1 to 3, more preferably 1, and the bond marked with * faces upwards from R^{4P} in formula (I);
   R^{1P} is selected from
a group —(CH₂)ₙ—, wherein n is an integer of 1 to 6, preferably 2 to 4, more preferably 2 or 4, and
a group *—(CH₂)_{q}—NH—C(O)—, wherein q is an integer of 1 to 5, preferably 1 to 3, more preferably 1, and the bond marked with * faces upwards from R^{1P} in formula (I);
   X^{1A} is selected from a covalent bond, an amide bond, an ether bond, a thioether bond, an ester bond, a thioester bond, a urea bond, a thiourea bond and an amine bond, preferably from an amide bond and an ester bond, and is more preferably an amide bond;
   L¹ is a divalent linking group;
   X^{1B} is selected from a covalent bond, an amide bond, an ether bond, a thioether bond, an ester bond, a thioester bond, a urea bond, a thiourea bond and an amine bond, preferably from an amide bond and an ester bond, and is more preferably an amide bond;
   R^{B} is a trivalent linking group;
   X^{2A} is selected from a covalent bond, an amide bond, an ether bond, a thioether bond, an ester bond, a thioester bond, a urea bond, a thiourea bond and an amine bond, preferably from an amide bond and an ester bond, and is more preferably an amide bond;
   L² is a divalent linking group;
   X^{2B} is selected from a covalent bond, an amide bond, an ether bond, a thioether bond, an ester bond, a thioester bond, a urea bond, a thiourea bond, and an amine bond, preferably from an amide bond and an ester bond, and is more preferably an amide bond;
   or —X^{2B}-L² is absent, such that X^{2A} is directly linked to R^{B}
   R^{CH} is a chelating group, optionally containing a chelated radioactive or non-radioactive cation;
   X^{3A} is selected from a covalent bond, an amide bond, an ether bond, a thioether bond, an ester bond, a thioester bond, a urea bond, a thiourea bond, an amine bond, and a dialkyl ammonium group —NR₂⁺—, wherein the groups R are each an alkyl group, preferably a methyl group, and is preferably selected from an amide bond, an ester bond, and a dialkyl ammonium group —NR₂⁺—, wherein the groups R are each an alkyl group, preferably a methyl group, and is more preferably an amide bond;
   L³ is a divalent linking group;
   X^{3B} is selected from a covalent bond, an amide bond, an ether bond, a thioether bond, an ester bond, a thioester bond, a urea bond, a thiourea bond and an amine bond, preferably from an amide bond and an ester bond, and is more preferably an amide bond;
   or —X^{3B}-L³ is absent, such that X^{3A} is directly linked to R^{B};
   R^{1S} and R^{2S} are independently a linear or branched C3 to C10 alkyl group, preferably R^{1S} and R^{2S} are selected from isopropyl and tert-butyl, and more preferably R^{1S} and R^{2S} are tert-butyl; and
   R^{3S} is a C1 to C20 hydrocarbon group which comprises one or more aromatic and/or aliphatic units, and which optionally comprises up to 3 heteroatoms selected from O and S, preferably R^{3S} is a C6 to C10 hydrocarbon group which comprises an aromatic ring and which may comprise one or more aliphatic units; more preferably R^{3S} is a phenylene group or a benzyl group carrying the Si atom shown in the formula attached as a substituent to its aromatic ring, and most preferably, R^{3S} is a phenylene group and the Si atom and X^{3A} are in a para-position to each other, or a benzyl group wherein the bond with X^{3A} is formed by the —CH₂— moiety at the benzyl group, and wherein the Si atom attached as a substituent to the aromatic ring and the —CH₂— moiety of the benzyl group are in a para-position to each other.
20. The ligand compound in accordance with item 19, wherein R^{1P} is CH₂, R^{3P} is CH₂, and R^{2P} is C.
21. The ligand compound in accordance with item 19 or 20, wherein
   R^{4P} is a group —(CH₂)ₘ—, wherein m is an integer of 2 to 6, preferably 2 to 4, more preferably 2, and
   R^{5P} is a group —(CH₂)ₙ—, wherein n is an integer of 1 to 6, preferably 2 to 4, more preferably 2 or 4.
22. The ligand compound in accordance with any of items 19 to 21, wherein m is 2.
23. The ligand compound in accordance with any of items 19 to 22, wherein n is 2 or 4.
24. The ligand compound in accordance with any of items 19 to 23, wherein R^{1S} and R^{2S} are both tert-butyl, and R^{3S} is a phenylene group and the Si atom and X^{3A} are in a para-position on the phenylene group, or a benzyl group carrying the Si atom shown in the formula attached as a substituent to its aromatic ring, wherein the bond with X^{3A} is formed with the —CH₂— moiety of the benzyl group, and the Si atom and the —CH₂— moiety of the benzyl group are in a para-position to each other.
25. The ligand compound in accordance with any of items 19 to 24, wherein
   X^{1A}, X^{1B}, and X^{2A} are independently selected from an ester bond and an amide bond,
   X^{3A} is selected from an amide bond, an ester bond, and a dialkyl ammonium group —NR₂*—, wherein the groups R are each an alkyl group, preferably a methyl group,
   X^{2B}-L² is absent, or X^{2B} is selected from an ester bond and an amide bond, and
   X^{3B}-L³ is absent, or X^{3B} is selected from an ester bond and an amide bond.
26. The ligand compound in accordance with item 25, wherein
   X^{1A}, X^{1B}, and X^{2A} are amide bonds,
   X^{3A} is selected from an amide bond and a dialkyl ammonium group —NR₂⁻—, wherein the groups R are each an alkyl group, preferably a methyl group, and X^{3A} is more preferably an amide bond,
   X^{2B}-L² is absent, or X²B is an amide bond, and
   X^{3B}-L³ is absent, or X^{3B} is an amide bond.
27. The ligand compound in accordance with any of items 19 to 26, wherein the chelating group comprises at least one of
   (i) a macrocyclic ring structure with 8 to 20 ring atoms of which 2 or more, preferably 3 or more, are selected from oxygen atoms and nitrogen atoms; and
   (ii) an acyclic, open chain chelating structure with 8 to 20 main chain atoms of which 2 or more, preferably 3 or more are heteroatoms selected from oxygen atoms and nitrogen atoms.
28. The ligand compound in accordance with any of items 19 to 27, wherein X^{2A} is an ester bond, an amide bond or a thiourea bond, more preferably an amide bond, wherein the chelating group R^{CH} is a residue of a chelating agent selected from bis(carboxymethyl)-1,4,8,11-tetraazabicyclo[6.6.2]hexadecane (CBTE2a), cyclohexyl-1,2-diaminetetraacetic acid (CDTA), 4-(1,4,8,11-tetraazacyclotetradec-1-yl)-methylbenzoic acid (CPTA), N′-[5-[acetyl(hydroxy)amino]pentyl]-N-[5-[[4-[5-aminopentyl-(hydroxy)amino]-4-oxobutanoyl]amino]pentyl]-N-hydroxybutandiamide (DFO), 4,11-bis(carboxymethyl)-1,4,8,11-tetraazabicycle[6.6.2]hexadecan (DO2A) 1,4,7,10-tetraazacyclododecan-N,N′,N″,N″′-tetraacetic acid (DOTA), 2-[1,4,7,10-tetraazacyclododecane-4,7,10-triacetic acid]-pentanedioic acid (DOTAGA or DOTA-GA), N,N′-dipyridoxylethylendiamine-N,N′-diacetate-5,5′-bis(phosphat) (DPDP), diethylenetriaminepentaacetic acid (DTPA), ethylenediamine-N,N′-tetraacetic acid (EDTA), ethyleneglykol-O,O-bis(2-aminoethyl)-N,N,N′,N′-tetraacetic acid (EGTA), N,N-bis(hydroxybenzyl)-ethylenediamine-N,N′-diacetic acid (HBED), hydroxyethyldiaminetriacetic acid (HEDTA), 1-(p-nitrobenzyl)-1,4,7,10-tetraazacyclo-decan-4,7,10-triacetate (HP-DOA3), 6-hydrazinyl-N-methylpyridine-3-n (HYNIC), 1,4,7-triazacyclononan-1-succinic acid-4,7-diacetic acid (NODASA), 1-(1-carboxy-3-carboxypropyl)-4,7-(carbooxy)-1,4,7-triazacyclononane (NODAGA), 1,4,7-triazacyclononanetriacetic acid (NOTA), 4,11-bis(carboxymethyl)-1,4,8,11-tetraaza-bicyclo[6.6.2]hexadecane (TE2A), 1,4,8,11-tetraazacyclododecane-1,4,8,11-tetra-acetic acid (TETA), terpyridine-bis(methyleneamine) tetraacetic acid (TMT), 1,4,7,10-tetraazacyclotridecan-N,N′,N″,N″′-tetraacetic acid (TRITA), and triethylenetetra-aminehexaacetic acid (TTHA), N,N′-bis[(6-carboxy-2-pyridil)methyl]-4,13-diaza-18-crown-6 (H₂macropa), 4-amino-4-{2-[(3-hydroxy-1,6-dimethyl-4-oxo-1,4-dihydro-pyridin-2-ylmethyl)-carbamoyl]-ethyl} heptanedioic acid bis-[(3-hydroxy-1,6-dimethyl-4-oxo-1,4-dihydro-pyridin-2-ylmethyl)-amide] (THP), 1,4,7-triazacyclononane-1,4,7-tris[methylene(2-carboxyethyl)phosphinic acid (TRAP), 2-(4,7,10-tris(2-amino-2-oxoethyl)-1,4,7,10-tetraazacyclododecan-1-yl)acetic acid (DO3AM), 1,4,7,10-tetraazacyclododecane-1,4,7,10-tetrakis[methylene(2-carboxyethylphosphinic acid)](DOTPI), and S-2-(4-isothiocyanatobenzyl)-1,4,7,10-tetraazacyclododecane tetraacetic acid;
   and the bond X^{2A} is formed using a functional group contained in the chelating agent, and wherein the chelating group optionally contains a chelated radioactive or non-radioactive cation.
29. The ligand compound of item 28, wherein the chelating agent contains a carboxy group, X^{2A} is an amide bond, and the amide bond X^{2A} is formed using the carboxy group contained in the chelating agent.
30. The ligand compound in accordance with item 28 or 29, wherein the chelating agent is selected from DOTA, DOTAGA and TRAP, more preferably from DOTA and DOTAGA.
31. The ligand compound in accordance with any of items 19 to 30, wherein —X^{2A}—R^{CH} is a group of the formula (XCH-1) or (XCH-2)

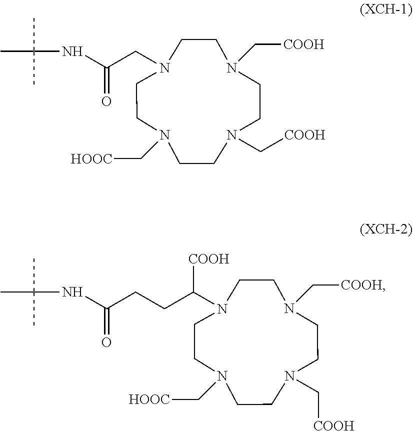

and is attached to the remainder of the compound via the bond marked by the dashed line, and wherein the chelating group optionally contains a chelated radioactive or non-radioactive cation.
32. The ligand compound in accordance with any of items 19 to 31, wherein the chelating group contains a chelated cation selected from the cations of ⁴³Sc, ⁴⁴Sc, ⁴⁷Sc, ⁵¹Cr, ^{52m}Mn, ⁵⁸Co, ⁵²Fe, ⁵⁶Ni, ⁵⁷Ni, ⁶²Cu, ⁶⁴Cu, ⁶⁷Cu, ⁶⁶Ga, ⁶⁸Ga, ⁶⁷Ga, ⁸⁹Zr, ⁹⁰Y, ⁸⁶Y, ⁹⁴mTc, ^{99m}Tc, ⁹⁷Ru, ¹⁰⁵Rh, ¹⁰⁹Pd, ¹¹¹Ag, ^{110m}In, ¹¹¹In, ^{113m}In, ^{114m}In, ^{117m}Sn, ¹²¹Sn, ¹²⁷Te, ¹⁴²Pr, ¹⁴³Pr, ¹⁴⁷Nd, ¹⁴⁹Gd, ¹⁴⁹Pm, ¹⁵¹Pm, ¹⁴⁹Tb, ¹⁵²Tb, ¹⁵⁵Tb, ¹⁵³Sm, ¹⁵⁶Eu, ¹⁵⁷Gd, ¹⁶¹Tb, ¹⁶⁴Tb, ¹⁶¹Ho, ¹⁶⁶Ho, ¹⁵⁷Dy, ¹⁶⁵Dy, ¹⁶⁶Dy, ¹⁶⁰Er, ¹⁶⁵Er, ¹⁶⁹Er, ¹⁷¹Er, ¹⁶⁶Yb, ¹⁶⁹Yb, ¹⁷⁵Yb, ¹⁶⁷Tm, ¹⁷²Tm, ^{nat}Lu, ¹⁷⁷Lu, ¹⁸⁶Re, ¹⁸⁸Re, ¹⁸⁸W, ¹⁹¹Pt, ^{195m}Pt, ¹⁹⁴Ir, ¹⁹⁷Hg, ¹⁹⁸Au, ¹⁹⁹Au, ²¹²Pb, ²⁰³Pb, ²¹¹At, ²¹²Bi, ²¹³Bi, ²²³Ra, ²²⁴Ra, ²²⁵Ac, and ²²⁷Th, or a cationic molecule comprising ¹⁸F, such as ¹⁸F-[AIF]²⁺.
33. The ligand compound in accordance with any of items 19 to 32, wherein the chelating group contains a chelated Ga cation or a chelated Lu cation.
34. The ligand compound in accordance with any of items 19 to 33, wherein the chelating group contains a chelated radioactive cation.
35. The ligand compound in accordance with item 34, wherein the chelating group contains a chelated ¹⁷⁷Lu cation.
36. The ligand compound in accordance with any of items 19 to 35, wherein L¹ comprises two or more subunits which are bonded to each other to form a chain of subunits between X^{1A} and X^{1B},
   wherein the bond(s) between the subunits in the chain of subunits is (are) independently selected for each occurrence from an amide bond, an ether bond, a thioether bond, an ester bond, a thioester bond, a urea bond, a thiourea bond and an amine bond.
37. The ligand compound in accordance with item 36, wherein the bond(s) between the subunits in the chain of subunits is (are) independently selected for each occurrence from an ether bond, an ester bond and an amide bond, and are more preferably an amide bond.
38. The ligand compound in accordance with item 36 or 37, wherein L¹ comprises 2 to 20 subunits, more preferably 2 to 15 subunits, still more preferably 2 to 12 subunits.
39. The ligand compound in accordance with any of items 19 to 38, wherein L¹ comprises 6 to 40 carbon atoms.
40. The ligand compound in accordance with any of items 36 to 39, wherein the subunits are independently selected from an alkanediyl unit, a cycloalkanediyl unit, a phenylene unit, an alkanediyl-cycloalkanediyl unit, an alkanediyl-cycloalkanediyl-alkanediyl unit, an alkanediyl-phenylene unit, and an alkanediyl-phenylene-alkanediyl unit, and wherein the chain of subunits is optionally substituted by one or more substituents independently selected from —OH, —OCH₃, —COOH, —COOCH₃, —NH₂, —CONH₂, —NHC(O)NH₂, —NHC(NH)NH₂, and aryl.
41. The ligand compound in accordance with item 40, wherein the chain of subunits of L¹ carries no substituent, or is substituted by one, two or three substituents independently selected from —OH, —OCH₃, —COOH, —COOCH₃, —NH₂, —CONH₂, —NHC(O)NH₂ and —NHC(NH)NH₂ and zero or one aryl substituent.
42. The ligand compound in accordance with any of items 36 to 39, wherein the subunits are independently selected from an alkanediyl unit, a cycloalkanediyl unit, a phenylene unit, an alkanediyl-cycloalkanediyl unit, an alkanediyl-cycloalkanediyl-alkanediyl unit, an alkanediyl-phenylene unit, and an alkanediyl-phenylene-alkanediyl unit, wherein the alkanediyl groups are linear alkanediyl groups, and wherein the chain of subunits is optionally substituted by one or more substituents independently selected from —OH, —OCH₃, —COOH, —COOCH₃, —NH₂, —CONH₂, —NHC(O)NH₂, —NHC(NH)NH₂, aralkyl and aryl.
43. The ligand compound in accordance with item 42, wherein the chain of subunits of L¹ carries no substituent, or is substituted by one, two or three substituents independently selected from —OH, —OCH₃, —COOH, —COOCH₃, —NH₂, —CONH₂, —NHC(O)NH₂ and —NHC(NH)NH₂ and zero or one substituent selected from aralkyl and aryl.
44. The ligand compound in accordance with any of items 38 to 41, wherein L¹ comprises not more than one subunit selected from the cycloalkanediyl unit, the phenylene unit, the alkanediyl-cycloalkanediyl unit, the alkanediyl-cycloalkanediyl-alkanediyl unit, the alkanediyl-phenylene unit, and the alkanediyl-phenylene-alkanediyl unit.
45. The ligand compound in accordance with any of items 40 to 44, wherein the chain of subunits comprises 6 to 40 carbon atoms, more preferably 6 to 30 carbon atoms, without carbon atoms contained in the optional substituents.
46. The ligand compound in accordance with any of items 19 to 45, wherein the group —X^{1A}-L¹-X^{1B}— in formula (I) is a group of any of the formulae (L-1) to (L-6):

*—NH—C(O)—R^{1L}—C(O)—NH—R^{2L}—NH—C(O)— (L-1)

*—C(O)—NH—R^{3L}—NH—C(O)—R^{4L}—C(O)—NH—R^{5L}—NH—C(O)— (L-2)

*—C(O)—NH—R^{6L}—NH—C(O)—R^{7L}—NH—C(O)—R^{8L}—NH—C(O)—R^{9L}—NH—C(O)— (L-3)

*—C(O)—NH—R^{10L}—NH—C(O)—R^{11L}—NH—C(O)— (L-4)

*—C(O)—NH—R^{12L}—NH—C(O)—R^{13L}—C(O)—NH—R^{14L}—NH—C(O)—R^{15L}—NH—C(O)— (L-5)

*—C(O)—NH—R^{16L}—C(O)—NH—R^{17L}—C(O)—NH—R^{18L}—C(O)—NH— (L-6)
wherein each of R^{1L} to R^{18L} is independently an alkanediyl group containing 1 to 8 carbon atoms, preferably a linear alkanediyl group containing 1 to 8 carbon atoms,
wherein each of R^{1L} to R^{18L} may be substituted by one or more substitutents independently selected from —OH, —OCH₃, —COOH, —COOCH₃, —NH₂, —CONH₂, —NHC(O)NH₂, and —NHC(NH)NH₂;
and wherein * marks the bond corresponding to the X^{1A} terminal bond of —X^{1A}-L¹-X^{1B}—.
47. The ligand compound in accordance with any of items 19 to 45, wherein the group —X^{1A}-L¹-X^{1B}— in formula (I) is a group of the formula (L-7):

*—NH—C(O)—R^{19L}—NH—C(O)—R^{20L}—NH—C(O)—R^{21L}—NH—C(O)— (L-7)
wherein R^{19L} is an alkanediyl group containing 1 to 8 carbon atoms, preferably a linear alkanediyl group containing 1 to 8 carbon atoms;
R^{20L} is an alkanediyl group containing 1 to 8 carbon atoms, a cycloalkanediyl group containing 3 to 6 carbon atoms or an alkanediyl-cycloalkanediyl group containing 5 to 8 carbon atoms,
wherein each of R^{19L} an R^{20L} may be substituted by one or more substituents independently selected from —OH, —OCH₃, —COOH, —COOCH₃, —NH₂, —CONH₂, —NHC(O)NH₂, —NHC(NH)NH₂, aryl and aralkyl;
R^{21L} is an alkanediyl group containing 2 to 26 carbon atoms, preferably a linear alkanediyl group containing 2 to 26 carbon atoms, wherein one or more —CH₂— groups may be replaced by —O—;
and wherein * marks the bond corresponding to the X^{1A} terminal bond of —X^{1A}-L¹-X^{1B}—.
48. The ligand compound in accordance with item 46,
   wherein, in formula (L-1), the total number of carbon atoms in R^{1L} and R^{2L} is 6 to 16, without carbon atoms contained in optional substituents that may be carried by the alkanediyl groups, and
   wherein in formula (L-2), the total number of carbon atoms in R^{3L} to R^{5L} is 6 to 16, without carbon atoms contained in optional substituents.
49. The ligand compound in accordance with any of items 19 to 48, wherein L¹ carries one or more substituents, and wherein at least one substituent is a substituent selected from —OH, —OCH₃, —COOH, —COOCH₃, —NH₂, —CONH₂, —NHC(O)NH₂, and —NHC(NH)NH₂, more preferably wherein at least one substituent is —COOH.
50. The ligand compound in accordance with item 46, wherein the group —X^{1A}-L¹-X^{1B}_in formula (I) is a group of the formula (L-8) or (L-9):

*—NH—C(O)—R^{22L}—C(O)—NH—R^{23L}—CH(COOH)—NH—C(O)— (L-8)

*—C(O)—NH—CH(COOH)—R^{24L}—NH—C(O)—R^{25L}—C(O)—NH—R^{26L}—CH(COOH)—NH—C(O)— (L-9)
wherein each of R^{22L} to R^{26L} is independently an alkanediyl group containing 1 to 6 carbon atoms, preferably a linear alkanediyl group containing 1 to 6 carbon atoms,
and wherein * marks the bond corresponding to the X^{1A} terminal bond of —X^{1A}-L¹-X^{1B}—.
51. The ligand compound in accordance with item 50,
   wherein the total number of carbon atoms in R^{22L} and R^{23L} is 6 to 12, more preferably 8 to 10, and
   wherein the total number of carbon atoms in R^{24L}, R^{25L} and R^{26L} is 6 to 12, more preferably 8 to 10.
52. The ligand compound in accordance with any of items 19 to 51,
   wherein —X^{2B}-L² is absent, or wherein the group L² is an alkanediyl group, preferably a linear alkanediyl group,
   which may be substituted by one or more substituents independently selected from —OH, —OCH₃, —COOH, —COOCH₃, —NH₂, —CONH₂, —NHC(O)NH₂ and —NHC(NH)NH₂;
   and wherein one or more covalent bonds between carbon atoms in the alkanedlyl group may be independently replaced by a bond selected from an amide bond, an ether bond, a thioether bond, an ester bond, a thioester bond, a urea bond, a thiourea bond and an amine bond, preferably by a bond selected from an ether bond, an ester bond and an amide bond, and more preferably by an amide bond.
53. The ligand compound in accordance with item 52, wherein if L² is present, the alkanediyl group contains 2 to 12 carbon atoms, more preferably 2 to 8 carbon atoms.
54. The ligand compound in accordance with item 52 or 53, wherein, if L² is present, it carries no substituent or is substituted by one or two substituents independently selected from —OH, —COOH, and —NH₂.
55. The ligand compound in accordance with any of items 52 to 54, wherein, if L² is present, zero, one, or two covalent bonds between carbon atoms are independently replaced by a bond selected from an amide bond, an ether bond, a thioether bond, an ester bond, a thioester bond, a urea bond, a thiourea bond, and an amine bond, preferably by a bond selected from an ether bond, an ester bond and an amide bond, and more preferably by an amide bond.
56. The ligand compound in accordance with any of items 19 to 55, wherein —X^{3B}-L³ is absent, or wherein the group L³ is an alkanediyl group, preferably a linear alkanediyl group,
   which may be substituted by one or more substituents independently selected from —OH, —OCH₃, —COOH, —COOCH₃, —NH₂, —CONH₂, —NHC(O)NH₂ and —NHC(NH)NH₂; and wherein one or more covalent bonds between carbon atoms in the alkanediyl group may be independently replaced by a bond selected from an amide bond, an ether bond, a thioether bond, an ester bond, a thioester bond, a urea bond, a thiourea bond and an amine bond, preferably by a bond selected from an ether bond, an ester bond and an amide bond, and more preferably by an amide bond.
57. The ligand compound in accordance with item 56, wherein, if L³ is present, the alkanediyl group contains 2 to 12 carbon atoms, more preferably 2 to 8 carbon atoms.
58. The ligand compound in accordance with item 56 or 57, wherein, if L³ is present, it carries no substituent or is substituted by one or two substituents independently selected from —OH, —COOH, and —NH₂.
59. The ligand compound in accordance with any of items 56 to 58, wherein, if L³ is present, zero, one, or two covalent bonds between carbon atoms are independently replaced by a bond selected from an amide bond, an ether bond, a thioether bond, an ester bond, a thioester bond, a urea bond and an amine bond, preferably by a bond selected from an ether bond, an ester bond and an amide bond, and more preferably by an amide bond.
60. The ligand compound in accordance with any of items 19 to 59, wherein —X^{2B}-L² is absent and —X^{3B}-L³ is absent.
61. The ligand compound in accordance with any of items 19 to 56, wherein —X^{3B}-L³-X^{3A}— is a group of the formula —NHC(O)—CH₂—N(CH₃)₂⁺—, and R^{3S} is a benzyl group wherein the bond with X^{3A} is formed by the —CH₂— moiety at the benzyl group, and wherein the Si atom attached as a substituent to the aromatic ring of the benzyl group and the —CH₂— moiety of the benzyl group are in a para-position to each other.
62. The ligand compound in accordance with any of items 19 to 61, wherein R^{B} in formula (I) is a group of the formula (B-1):

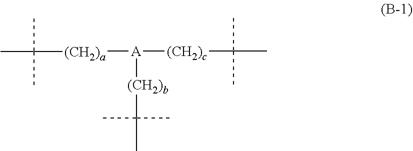

wherein
A is selected from N, CR^{B1} wherein R^{B1} is H or C₁-C₆ alkyl, and a 5 to 7 membered carbocyclic or heterocyclic group; preferably A is selected from N and CH, and more preferably A is CH;
the bond marked by the dashed line at (CH₂)ₐ is formed with X^{1B}, and a is an integer of 0 to 4, preferably 0 or 1, and most preferably 0;
the bond marked by the dashed line at (CH₂)_{b} is formed with X^{3B}, if present, and otherwise with X^{3A}, and b is an integer of 0 to 4, preferably of 0 to 2, and more preferably 0 or 1;
and the bond marked by the dashed line at (CH₂)_{c} is formed with X^{2B}, if present, and otherwise with X^{2A}, and c is an integer of 0 to 4, preferably of 0 to 2, and more preferably 0 or 1.
63. The ligand compound in accordance with item 62, wherein R^{B} in formula (I) is a group of the formula (B-2):

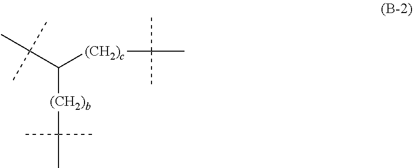

wherein
the bond marked by the dashed line at (CH₂)_{b} is formed with X^{3B}, if present, and otherwise with X^{3A}, and b is an integer of 0 to 4, preferably of 0 to 2, and more preferably 0 or 1;
the bond marked by the dashed line at (CH₂)_{c} is formed with X^{2B}, if present, and otherwise with X^{2A}, and c is an integer of 0 to 4, preferably of 0 to 2, and more preferably 0 or 1,
and the remaining bond marked by the dashed line is formed with X^{1B}.
64. The ligand compound in accordance with any of items 19 to 63, which is a compound of formula (II) or a pharmaceutically acceptable salt thereof

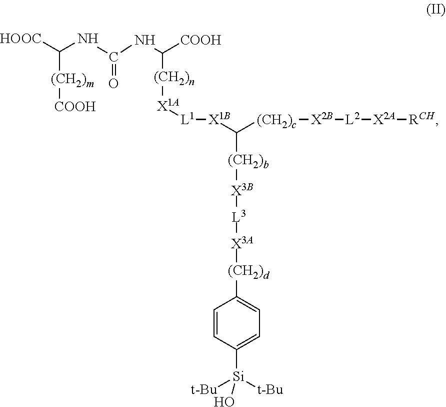

wherein, in formula (II), m, n, b, c, X^{1A}, L¹, X^{1B}, X^{2B}, L², X^{2A}, R^{CH}, X^{3A}, L³ and X^{3B} are defined as in the preceding items 19 to 62, and d is 0 or 1.
65. A therapeutic composition comprising or consisting of one or more ligand compounds in accordance with any of items 1 to 64.
66. A ligand compound in accordance with any of items 1 to 64 or a therapeutic composition in accordance with item 65 for use in a therapeutic method.
67. The ligand compound in accordance with any of items 1 to 64 or the therapeutic composition in accordance with item 65 for use in treating cancer.
68. The ligand compound or the pharmaceutical composition for use in accordance with item 67, wherein the cancer is prostate cancer.
69. The ligand compound in accordance with any of items 1 to 64 or the pharmaceutical composition in accordance with item 65 for use in treating neoangiogenesis.
70. A diagnostic composition comprising or consisting of one or more ligand compounds in accordance with any of items 1 to 64.
71. A ligand compound in accordance with any of items 1 to 64 or a diagnostic composition in accordance with item 70 for use in a method of diagnosis in vivo of a disease or disorder.
72. The ligand compound in accordance with any of items 1 to 64 or the diagnostic composition in accordance with item 70 for use in a method of diagnosis in vivo of cancer.
73. The ligand compound or the diagnostic composition for use in accordance with item 72, wherein the cancer is prostate cancer.
74. The ligand compound in accordance with any of items 1 to 64 or the diagnostic composition in accordance with item 70 for use in a method of diagnosis in vivo of neoangiogenesis.

As noted above, the present invention provides a ligand compound, comprising: (a) a targeting group, (b) one or more chelating groups, optionally containing a chelated radioactive or non-radioactive cation, and (c) a group carrying an Si—OH functional moiety. As will be understood, these groups are combined within the same compound, i.e. within one molecule.

The group carrying an Si—OH functional moiety is a group comprising a silicon atom, and a hydroxy substituent bound via a covalent bond to the silicon atom. Favorably, the Si—OH bond is stable e.g. under in vivo conditions when the ligand compounds in accordance with the invention are administered to a subject, and also under the reaction conditions used for forming a chelate containing a radioactive or non-radioactive cation with the ligand compounds in accordance with the invention. Further substituents attached to the silicon atom are not particularly restricted, typically these substituents are hydrocarbyl substituents.

Preferably, the group carrying an Si—OH functional moiety is a group of formula (S-1)

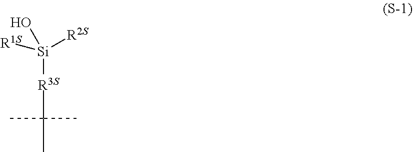

wherein
R^{1S} and R^{2S} are independently a linear or branched C3 to C10 alkyl group, preferably R^{1S} and R^{2S} are selected from isopropyl and tert-butyl, and more preferably R^{1S} and R^{2S} are tert-butyl;
R^{3S} is a C1 to C20 hydrocarbon group which comprises one or more aromatic and/or aliphatic units, and which optionally comprises up to 3 heteroatoms independently selected from O, N and S, preferably R^{3S} is a C6 to C10 hydrocarbon group which comprises an aromatic ring, such as a phenyl ring, and which may comprise one or more aliphatic units; more preferably R^{3S} is a phenylene group or a benzyl group carrying the Si atom shown in the formula attached as a substituent to its aromatic ring, and most preferably, R^{3S} is a phenylene group, and the Si atom and the bond marked by the dashed line are in a para-position to each other, or a benzyl group wherein the bond with the dashed line is formed by the —CH₂— moiety at the benzyl group, and the Si atom attached as a substituent to the aromatic ring and the —CH₂— moiety of the benzyl group are in a para-position to each other;
and wherein the group carrying an Si—OH functional moiety of formula (S-1) is attached to the remainder of the compound via the bond marked by the dashed line.

As will be understood, the definition according to which R^{3S} is a C1 to C20 hydrocarbon group which comprises one or more aromatic and/or aliphatic units, encompasses the case that R^{3S} is an aromatic group, that R^{3S} is an aliphatic group, and that R^{3S} combines an one or more aromatic groups, and one or more aliphatic groups, while the limitation in terms of the number of carbon atoms needs to be observed. As noted above, R^{3S} optionally comprises up to 3 heteroatoms independently selected from O, N and S, which may be interspersed in the hydrocarbon group between carbon atoms.

More preferably, the group carrying an Si—OH functional moiety is a group of formula (S-2) or (S-3)

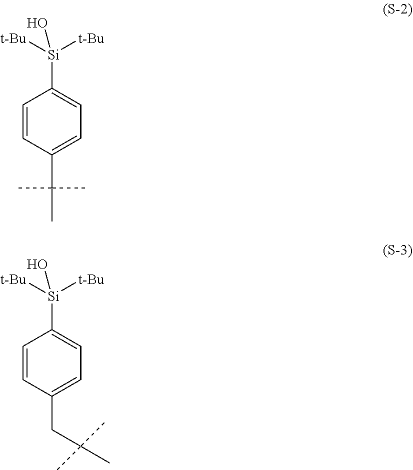

wherein t-Bu indicates a tert-butyl group,
and wherein the group carrying an Si—OH functional moiety of formula (S-2) or (S-3) is attached to the remainder of the compound via the bond marked by the dashed line.

As discussed herein above, the compounds in accordance with the present invention are suitable as radiopharmaceuticals or precursors thereof, in particular as radiotherapeutics or precursors thereof, or as radiodiagnostics or precursors thereof. Accordingly, a targeting group in accordance with the present invention is generally a group that is capable of binding to a therapeutic or diagnostic target. A diagnostic target shall mean any target which allows for binding a disease or the risk of developing a disease. A therapeutic target is a target which allows for treating or preventing a disease or disease state, if targeted. As will be understood by the skilled reader, the two terms are not mutually exclusive, i.e. a target may be of interest both for therapy and for diagnosis.

The therapeutic or diagnostic target is preferably a tumor antigen. A tumor antigen is an antigenic substance produced by tumor cells, i.e., it triggers an immune response in the host. Tumor antigens are useful tumor markers in identifying tumor cells with diagnostic tests. Tumor antigens are also potential targets for a cancer therapy. A tumor antigen may be a tumor-specific antigens (TSA), which is present only on tumor cells and not on any other cells or a tumor-associated antigen (TAA), which is present on some tumor cells and also on some normal cells.

Non-limiting but preferred examples of tumor antigens are PSMA (prostate tumor). alphafetoprotein (AFP) (germ cell tumors and hepatocellular carcinoma), carcinoembryonic antigen (CEA) (bowel cancers, occasional lung or breast cancer), CA-125 (ovarian cancer), MUC-1 (breast cancer), epithelial tumor antigen (ETA) (breast cancer), tyrosinase (malignant melanoma), melanoma-associated antigen (MAGE) (malignant melanoma) and abnormal products of ras, p53 (various tumors).

Among this list of tumor antigens, PSMA is preferred.

The targeting group is therefore preferably a group that is capable of binding to a tumor antigen, more preferably a PSMA binding group. The PSMA binding group is a group which is able to bind with high affinity to PSMA. Suitable PSMA binding groups (also referred to as PSMA ligands) are described in the literature as discussed above, and can be incorporated as targeting group into the ligand compound in accordance with the present invention.

Preferably, the targeting group is a PSMA binding group of formula (P-1) or a pharmaceutically acceptable salt thereof

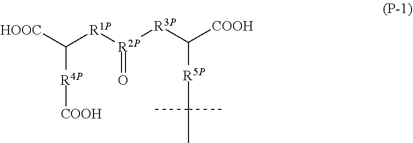

wherein:
R^{1P} is CH₂, NH or O, preferably NH;
R^{3P} is CH₂, NH or O, preferably NH;
R^{2P} is C or P(OH), preferably C;
R^{4P} is selected from
a group —(CH₂)ₘ—, wherein m is an integer of 2 to 6, preferably 2 to 4, more preferably 2, and
a group *—(CH₂)ₚ—NH—C(O)—, wherein p is an integer of 1 to 5, preferably 1 to 3, more preferably 1, and the bond marked with * faces upwards from R^{4P} in formula (P-1);
R^{5P} is selected from
a group —(CH₂)ₙ—, wherein n is an integer of 1 to 6, preferably 2 to 4, more preferably 2 or 4, and
a group *—(CH₂)_{q}—NH—C(O)—, wherein q is an integer of 1 to 5, preferably 1 to 3, more preferably 1, and the bond marked with * faces upwards from R^{5P} in formula (P-1);
and wherein the PSMA binding group is attached to the remainder of the compound via the bond marked by the dashed line.

The PSMA binding group is more preferably a group of formula (P-2) or a pharmaceutically acceptable salt thereof

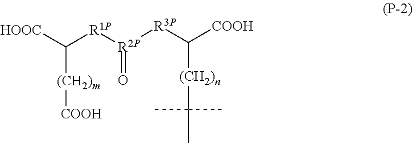

wherein:
m is an integer of 2 to 6, preferably 2 to 4, more preferably 2;
n is an integer of 1 to 6, preferably 2 to 4, more preferably 2 or 4;
R^{1P} is CH₂, NH or O, preferably NH;
R^{3P} is CH₂, NH or O, preferably NH;
R^{2P} is C or P(OH), preferably C;
and wherein the PSMA binding group is attached to the remainder of the compound via the bond marked by the dashed line.

As will be understood, in formula (P-1) and (P-2) it is preferred that R^{1P} is NH, R^{3P} is NH, and R^{2P} is C, and it is still more preferred that R^{1P} is NH, R^{3P} is NH, and R^{2P} is C, and m is 2.

It is particularly preferred that the PSMA binding group is a group of formula (P-3), or a pharmaceutically acceptable salt thereof:

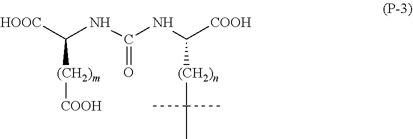

wherein:
m is an integer of 2 to 6, preferably 2 to 4, more preferably 2;
n is an integer of 2 to 6, preferably 2 to 4, more preferably 2 or 4;
and wherein the PSMA binding group is attached to the remainder of the compound via the bond marked by the dashed line.

Also in formula (P-3), it is further preferred that m is 2, and still further preferred that m is 2 and n is 2.

The ligand compound further comprises one or more, such as one, two or three, preferably one, chelating group(s). The chelating group(s) are suitable to bind, by chelate bonding, a cation. Thus, the chelating group(s) contained in the compounds in accordance with the invention optionally contain(s) a chelated radioactive or non-radioactive cation, i.e. the compounds in accordance with the invention may comprise a chelate formed by the chelating group and a chelated radioactive or non-radioactive cation. Metal- or cation-chelating compounds, e.g. macrocyclic or acyclic compounds, which are suitable to provide a chelating group, are well-known in the art and available from a number of manufacturers. While the chelating group in accordance with the present invention is not particularly limited, it is understood that numerous moieties can be used in an off-the-shelf manner by a skilled person without further ado.

A preferred chelating group comprises at least one of the following (i) and (ii).
(i) A macrocyclic ring structure with 8 to 20 ring atoms of which 2 or more, more preferably 3 or more, are selected from oxygen atoms or nitrogen atoms. Preferably, 6 or less ring atoms are selected from oxygen atoms or nitrogen atoms. Especially preferred is that 3 or 4 ring atoms are nitrogen atoms or oxygen atoms. Among the oxygen and nitrogen atoms, preference is given to the nitrogen atoms. In combination with the macrocyclic ring structure, the preferred chelating group may comprise 2 or more, such as 2 to 6, preferably 2 to 4, carboxy groups and/or hydroxy groups. Among the carboxy groups and the hydroxy groups, preference is given to the carboxy groups.
(ii) An acyclic, open chain chelating structure with 8 to 20 main chain (back bone) atoms of which 2 or more, more preferably 3 or more are heteroatoms selected from oxygen atoms or nitrogen atoms. Preferably, 6 or less back bone atoms are selected from oxygen atoms or nitrogen atoms. Among the oxygen and nitrogen atoms, preference is given to the nitrogen atoms. More preferably, the open chain chelating structure is a structure which comprises a combination of 2 or more, more preferably 3 or more heteroatoms selected from oxygen atoms or nitrogen atoms, and 2 or more, such as 2 to 6, preferably 2 to 4, carboxy groups and/or hydroxy groups. Among the carboxy groups and the hydroxy groups, preference is given to the carboxy groups.

More preferably, the chelating group is a residue of a chelating agent selected from bis(carboxymethyl)-1,4,8,11-tetraazabicyclo[6.6.2]hexadecane (CBTE2a), cyclohexyl-1,2-diaminetetraacetic acid (CDTA), 4-(1,4,8,11-tetraazacyclotetradec-1-yl)-methylbenzoic acid (CPTA), N′-[5-[acetyl(hydroxy)amino]pentyl]-N-[5-[[4-[5-aminopentyl-(hydroxy)amino]-4-oxobutanoyl]amino]pentyl]-N-hydroxybutandiamide (DFO), 4,11-bis(carboxymethyl)-1,4,8,11-tetraazabicycle[6.6.2]hexadecan (DO2A) 1,4,7,10-tetraazacyclododecan-N,N′,N,N″′-tetraacetic acid (DOTA), 2-[1,4,7,10-tetraazacyclododecane-4,7,10-triacetic acid]-pentanedioic acid (DOTAGA), N,N′-dipyridoxylethylendiamine-N,N′-diacetate-5,5′-bis(phosphat) (DPDP), diethylenetriaminepentaacetic acid (DTPA), ethylenediamine-N,N′-tetraacetic acid (EDTA), ethyleneglykol-O,O-bis(2-aminoethyl)-N,N,N′,N′-tetraacetic acid (EGTA), N,N-bis(hydroxybenzyl)-ethylenediamine-N,N′-diacetic acid (HBED), hydroxyethyldiaminetriacetic acid (HEDTA), 1-(p-nitrobenzyl)-1,4,7,10-tetraazacyclodecan-4,7,10-triacetate (HP-DOA3), 6-hydrazinyl-N-methylpyridine-3-carboxamide (HYNIC), 1,4,7-triazacyclononan-1-succinic acid-4,7-diacetic acid (NODASA), 1-(1-carboxy-3-carboxypropyl)-4,7-(carboxy)-1,4,7-triazacyclononane (NODAGA), 1,4,7-triazacyclononanetriacetic acid (NOTA), 4,11-bis(carboxymethyl)-1,4,8,11-tetraazabicyclo[6.6.2]hexadecane (TE2A), 1,4,8,11-tetraazacyclododecane-1,4,8,11-tetraacetic acid (TETA), terpyridine-bis(methyleneamine) tetraacetic acid (TMT), 1,4,7,10-tetraazacyclotridecan-N,N′,N″,N″′-tetraacetic acid (TRITA), and triethylenetetraaminehexaacetic acid (TTHA), N,N′-bis[(6-carboxy-2-pyridil)methyl]-4,13-diaza-18-crown-6 (H₂macropa), 4-amino-4-{2-[(3-hydroxy-1,6-dimethyl-4-oxo-1,4-dihydro-pyridin-2-ylmethyl)-carbamoyl]-ethyl} heptanedioic acid bis-[(3-hydroxy-1,6-dimethyl-4-oxo-1,4-dihydro-pyridin-2-ylmethyl)-amide] (THP), 1,4,7-triazacyclononane-1,4,7-tris[methylene(2-carboxyethyl)phosphinic acid (TRAP), 2-(4,7,10-tris(2-amino-2-oxoethyl)-1,4,7,10-tetraazacyclododecan-1-yl)acetic acid (DO3AM), 1,4,7,10-tetraazacyclododecane-1,4,7,10-tetrakis[methylene(2-carboxyethylphosphinic acid)] (DOTPI) and S-2-(4-isothiocyanatobenzyl)-1,4,7,10-tetraazacyclododecane tetraacetic acid, and from pharmaceutically acceptable salts thereof. As noted above, the chelating group optionally contains a chelated radioactive or non-radioactive cation.

The residue of the above chelating agents is typically bound to the remainder of the compound via an ester bond (i.e. —C(O)—O—), an amide bond (i.e. preferably —C(O)—NH—), or a thiourea bond (—NH—C(S)—NH—), preferably via an amide bond. The ester bond or amide bond can be formed using a carboxy group or an amide group contained in the chelating agent. The thiourea bond can be formed using an isothiocycanato group provided by the chelating agent. In other words, the ligand compound preferably comprises a residue which is derived from one of the above preferred chelating agents by incorporating the chelating agent into the compound by forming an ester bond, an amide bond or a thiourea bond, preferably an amide bond, using a carboxy group or an amide group contained in the chelating agent. As will be understood by the skilled reader a bond can be formed using a functional group by reacting the functional group, e.g. a carboxy group or an activated derivative thereof, with a suitable reaction partner, e.g. an amino group.

Further preferred for providing the chelating group in the ligand compound in accordance with the invention is a chelating agent which contains a carboxy group, and the residue obtained from the chelating agent is bound to the remainder of the compound via an amide bond that is formed using the carboxy group.

More preferred among these chelating agents are DOTA, DOTAGA (also known as DOTA-GA), and TRAP, and still more preferred are DOTA and DOTAGA.

In line with the above, the chelating group is preferably selected from a group of the formula (CH-1) or (CH-2), or a pharmaceutically acceptable salt thereof

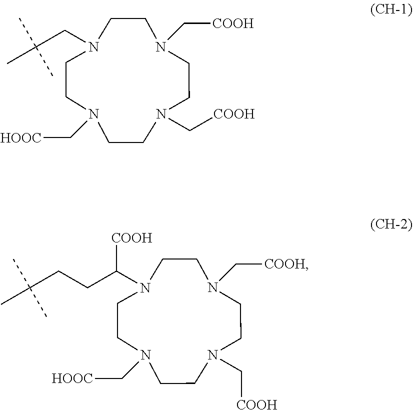

which chelating group is attached by the bond marked by the dashed line to the remainder of the compound, preferably via an ester or an amide bond, more preferably an amide bond, and optionally contains a chelated radioactive or non-radioactive cation.

The chelating group optionally contains a chelated radioactive or non-radioactive cation. The cation may be a metal cation or a non-metallic cation. Preferably, the cation is a radioactive cation. It is also preferred that the cation is a metal cation. Thus, radioactive metal cations are particularly preferred.

Exemplary cations which may be contained as chelated cations in the chelating group are selected from the cations of ⁴³Sc, ⁴⁴Sc, ⁴⁷Sc, ⁵¹Cr, ^{52m}Mn, ⁵⁸Co, ⁵²Fe, ⁵⁶Ni, ⁵⁷Ni, ⁶²Cu, ⁶⁴Cu, ⁶⁷Cu, ⁶⁶Ga, ⁶⁸Ga, ⁶⁷Ga, ⁸⁹Zr, ⁹⁰Y, ⁸⁶Y, ^{94m}Tc, ^{99m}Tc, ⁹⁷Ru, ¹⁰⁵Rh, ¹⁰⁹Pd, ¹¹¹Ag, ^{110m}In, ¹¹¹In, ^{113m}In, ^{114m}In, ^{117m}Sn, ¹²¹Sn, ¹²⁷Te, ¹⁴²Pr, ¹⁴³Pr, ¹⁴⁷Nd, ¹⁴⁹Gd, ¹⁴⁹Pm, ¹⁵¹Pm, ¹⁴⁹Tb, ¹⁵²Tb, ¹⁵⁵Tb, ¹⁵³Sm, ¹⁵⁶Eu, ¹⁵⁷Gd, ¹⁶¹Tb, ¹⁶⁴Tb, ¹⁶¹Ho, ¹⁶⁶Ho, ¹⁵⁷Dy, ¹⁶⁵Dy, ¹⁶⁶Dy, ¹⁶⁰Er, ¹⁶⁵Er, ¹⁶⁹Er, ¹⁷¹Er, ¹⁶⁶Yb, ¹⁶⁹Yb, ¹⁷⁵Yb, ¹⁶⁷Tm, ¹⁷²Tm, ^{nat}Lu, ¹⁷⁷Lu, ¹⁸⁶Re, ¹⁸⁸Re, ¹⁸⁸W, ¹⁹¹Pt, ^{195m}Pt, ¹⁹⁴Ir, ¹⁹⁷Hg, ¹⁹⁸Au, ¹⁹⁹Au, ²¹²Pb, ²⁰³Pb, ²¹¹At, ²¹²Bi, ²¹³Bi, ²²³Ra, ²²⁴Ra, ²²⁵Ac, and ²²⁷Th, or a cationic molecule comprising ¹⁸F, such as ¹⁸F-[AIF]²⁺.

Preferred cations which may be contained as chelated cations in the chelating groups are Ga cations or Lu cations, e.g. ⁶⁸Ga or ¹⁷⁷Lu.

A particularly preferred example of a cation which may be contained as chelated cation in the chelating group is a cation of ¹⁷⁷Lu.

In line with the above, the ligand compound in accordance with the invention is preferably a PSMA ligand compound of formula (I) or a pharmaceutically acceptable salt thereof:

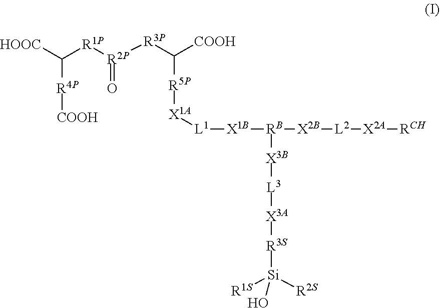

wherein, in formula (I),
   R^{1P} is CH₂, NH or O, preferably NH;
   R^{3P} is CH₂, NH or O, preferably NH;
   R^{2P} is C or P(OH), preferably C;
   R^{4P} is selected from
a group —(CH₂)ₘ—, wherein m is an integer of 2 to 6, preferably 2 to 4, more preferably 2, and
a group *—(CH₂)ₚ—NH—C(O)—, wherein p is an integer of 1 to 5, preferably 1 to 3, more preferably 1, and the bond marked with * faces upwards from R^{4P} in formula (I);
   R^{5P} is selected from
a group —(CH₂)ₙ—, wherein n is an integer of 1 to 6, preferably 2 to 4, more preferably 2 or 4, and
a group *—(CH₂)_{q}—NH—C(O)—, wherein q is an integer of 1 to 5, preferably 1 to 3, more preferably 1, and the bond marked with * faces upwards from R^{5P} in formula (I);
X^{1A} is selected from a covalent bond, an amide bond, an ether bond, a thioether bond, an ester bond, a thioester bond, a urea bond, a thiourea bond and an amine bond, preferably from an amide bond and an ester bond, and is more preferably an amide bond;
L¹ is a divalent linking group;
X^{1B} is selected from a covalent bond, an amide bond, an ether bond, a thioether bond, an ester bond, a thioester bond, a urea bond, a thiourea bond and an amine bond, preferably from an amide bond and an ester bond, and is more preferably an amide bond;
R^{B} is a trivalent linking group;
X^{2A} is selected from a covalent bond, an amide bond, an ether bond, a thioether bond, an ester bond, a thioester bond, a urea bond, a thiourea bond and an amine bond, preferably from an amide bond and an ester bond, and is more preferably an amide bond;
L² is a divalent linking group;
X^{2B} is selected from a covalent bond, an amide bond, an ether bond, a thioether bond, an ester bond, a thioester bond, a urea bond, a thiourea bond and an amine bond, preferably from an amide bond and an ester bond, and is more preferably an amide bond;
or —X^{2B}-L² is absent, such that X^{2A} is directly linked to R^{B}
R^{CH} is a chelating group, optionally containing a chelated radioactive or non-radioactive cation;
X^{3A} is selected from a covalent bond, an amide bond, an ether bond, a thioether bond, an ester bond, a thioester bond, a urea bond, a thiourea bond, an amine bond and a dialkyl ammonium group —NR₂₊—, wherein the groups R are each an alkyl group, preferably a methyl group, and is preferably selected from an amide bond, an ester bond, and a dialkyl ammonium group —NR₂⁺—, wherein the groups R are each an alkyl group, preferably a methyl group, and is more preferably an amide bond;
L³ is a divalent linking group;
X^{3B} is selected from a covalent bond, an amide bond, an ether bond, a thioether bond, an ester bond, a thioester bond, a urea bond, a thiourea bond and an amine bond, preferably from an amide bond and an ester bond, and is more preferably an amide bond;
or —X^{3B}-L³ is absent, such that X^{3A} is directly linked to R^{B};
R^{1S} and R^{2S} are independently a linear or branched C3 to C10 alkyl group, preferably R^{1S} and R^{2S} are selected from isopropyl and tert-butyl, and more preferably R^{1S} and R^{2S} are tert-butyl; and
R^{3S} is a C1 to C20 hydrocarbon group which comprises one or more aromatic and/or aliphatic units, and which optionally comprises up to 3 heteroatoms selected from O, N and S, preferably R^{3S} is a C6 to C10 hydrocarbon group which comprises an aromatic ring and which may comprise one or more aliphatic units. More preferably R^{3S} is selected from (i) a phenylene group and (ii) a benzyl group, which benzyl group carries the Si atom shown in the formula attached as a substituent to its aromatic ring. Most preferably, R^{3S} is selected from (i) a phenylene group, and the Si atom and X^{3A} attached in the formula to R^{3S} are in para-position to each other on the phenylene group, and (ii) a benzyl group wherein the bond with X^{3A} is formed by the —CH₂— moiety at the benzyl group, and wherein the Si atom attached as a substituent to the aromatic ring and the —CH₂— moiety of the benzyl group are in a para-position to each other on the aromatic ring of the benzyl group.

Unless specifically indicated otherwise by the definitions provided for the variables in the formula, it will be understood that the explanations provided for the components targeting group (a), the chelating group (c) and the group carrying an Si—OH (c) above continue to apply for the ligand compound of formula (I) and its preferred embodiments.

Moreover, as indicated above, the ligand compound in accordance with the invention is preferably a compound of formula (I) or a pharmaceutically acceptable salt thereof. Thus, to the extent that any group or atom used in the definition of a variable of formula (I) allows such a salt to be formed, it will be understood that this salt is also encompassed as a ligand compound in accordance with the invention.

As will be understood by the skilled reader, variables or moieties in a formula linked by an amide bond are linked by the group —C(O)—NR—, wherein R is H or a hydrocarbon group, preferably H or C1-6 alkyl, and most preferably H, variables or moieties in a formula linked by an ether bond are linked by the group —O—, variables or moieties in a formula linked by a thioether bond are linked by the group —S—, variables or moieties in a formula linked by an ester bond are linked by the group —C(O)—O—, variables or moieties in a formula linked by an thioester bond are linked by the group —C(O)—S—, variables or moieties in a formula linked by a urea bond are linked by the group —NH—C(O)—NH—, variables or moieties in a formula linked by a thiourea bond are linked by the group —NH—C(S)—NH— and variables or moieties in a formula linked by an amine bond are linked by the group —NR—, wherein R is H or an hydrocarbon group, preferably H or C₁₋₆ alkyl, and most preferably H.

In formula (I), it is preferred that R^{1P} is NH, R^{3P} is NH, and R^{2P} is C. It is further preferred that R^{1P} is NH, R^{3P} is NH, R^{2P} is C.

R^{4P} is preferably the group —(CH₂)ₘ—, wherein m is an integer of 2 to 6, preferably 2 to 4, more preferably 2, and

R^{5P} is preferably the group —(CH₂)ₙ—, wherein n is an integer of 1 to 6, preferably 2 to 4, more preferably 2 or 4.

m is preferably 2, and n is preferably 2 or 4.

Thus, it is further preferred that R^{1P} is NH, R^{3P} is NH, R^{2P} is C, R^{4P} is the group —(CH₂)ₘ—, wherein m is 2, and R^{5P} is the group —(CH₂)ₙ—, wherein n is 2 or 4.

As regards R^{1S}, R^{2S} and R^{3S}, it is further preferred that R^{1S} and R^{2S} are both tert-butyl, and that R^{3S} is selected from (i) a phenylene group and the Si atom and X^{3A} are in a para-position on the phenylene group, and (ii) a benzyl group carrying the Si atom shown in the formula attached as a substituent to its aromatic ring, wherein the bond with X^{3A} is formed by the —CH₂— moiety at the benzyl group, and the Si atom and the —CH₂— moiety of the benzyl group are in a para-position to each other.

X^{1A}, X^{1B}, and X^{2A} are preferably independently selected from an ester bond and an amide bond, and it is further preferred that all of X^{1A}, X^{1B}, and X^{2A} are amide bonds.

X^{3A} is preferably selected from an amide bond, an ester bond, and a dialkyl ammonium group —NR₂⁺—, wherein the groups R are each an alkyl group. As alkyl groups, methyl groups are preferred. It is further preferred that X^{3A} is an amide bond.

Moreover, it is preferred that —X^{2B}-L² is absent, or that X^{2B} is selected from an ester bond and an amide bond, more preferably —X^{2B}-L² is absent, or X²B is an amide bond.

Likewise, it is preferred that —X^{3B}-L³ is absent, or that X^{3B} is selected from an ester bond and an amide bond, and it is more preferred that —X³B-L³ is absent, or X^{3B} is an amide bond.

A preferred chelating group R^{CH} comprises at least one of the following (i) and (ii).
(i) A macrocyclic ring structure with 8 to 20 ring atoms of which 2 or more, more preferably 3 or more, are selected from oxygen atoms or nitrogen atoms. Preferably, 6 or less ring atoms are selected from oxygen atoms or nitrogen atoms. Especially preferred is that 3 or 4 ring atoms are nitrogen atoms or oxygen atoms. Among the oxygen and nitrogen atoms, preference is given to the nitrogen atoms. In combination with the macrocyclic ring structure, the preferred chelating group may comprise 2 or more, such as 2 to 6, preferably 2 to 4, carboxy groups and/or hydroxy groups. Among the carboxy groups and the hydroxy groups, preference is given to the carboxy groups.
(ii) An acyclic, open chain chelating structure with 8 to 20 main chain (back bone) atoms of which 2 or more, more preferably 3 or more are heteroatoms selected from oxygen atoms or nitrogen atoms, Preferably, 6 or less back bone atoms are selected from oxygen atoms or nitrogen atoms. Among the oxygen and nitrogen atoms, preference is given to the nitrogen atoms. More preferably, the open chain chelating structure is a structure which comprises a combination of 2 or more, more preferably 3 or more heteroatoms selected from oxygen atoms or nitrogen atoms, and 2 or more, such as 2 to 6, preferably 2 to 4, carboxy groups and/or hydroxy groups. Among the carboxy groups and the hydroxy groups, preference is given to the carboxy groups.

It is more preferred that X^{2A} is an ester bond, an amide bond or a thiourea bond, still more preferably an amide bond, and the chelating group R^{CH} is a residue of a chelating agent selected from bis(carboxymethyl)-1,4,8,11-tetraazabicyclo[6.6.2]hexadecane (CBTE2a), cyclohexyl-1,2-diaminetetraacetic acid (CDTA), 4-(1,4,8,11-tetraazacyclotetradec-1-yl)-methylbenzoic acid (CPTA), N′-[5-[acetyl(hydroxy)amino]pentyl]-N-[5-[[4-[5-aminopentyl-(hydroxy)amino]-4-oxobutanoyl]amino]pentyl]-N-hydroxybutandiamide (DFO), 4,11-bis(carboxymethyl)-1,4,8,11-tetraazabicycle[6.6.2]hexadecan (DO2A) 1,4,7,10-tetraazacyclododecan-N,N′,N″,N″′-tetraacetic acid (DOTA), 2-[1,4,7,10-tetraazacyclododecane-4,7,10-triacetic acid]-pentanedioic acid (DOTAGA), N,N′-dipyridoxylethylendiamine-N,N′-diacetate-5,5′-bis(phosphat) (DPDP), diethylenetriaminepentaacetic acid (DTPA), ethylenediamine-N,N′-tetraacetic acid (EDTA), ethyleneglykol-O,O-bis(2-aminoethyl)-N,N,N′,N′-tetraacetic acid (EGTA), N,N-bis(hydroxybenzyl)-ethylenediamine-N,N′-diacetic acid (HBED), hydroxyethyldiaminetriacetic acid (HEDTA), 1-(p-nitrobenzyl)-1,4,7,10-tetraazacyclodecan-4,7,10-triacetate (HP-DOA3), 6-hydrazinyl-N-methylpyridine-3-carboxamide (HYNIC), 1,4,7-triazacyclononan-1-succinic acid-4,7-diacetic acid (NODASA), 1-(1-carboxy-3-carboxypropyl)-4,7-(carbooxy)-1,4,7-triazacyclononane (NODAGA), 1,4,7-triazacyclononanetriacetic acid (NOTA), 4,11-bis(carboxymethyl)-1,4,8,11-tetraazabicyclo[6.6.2]hexadecane (TE2A), 1,4,8,11-tetraazacyclododecane-1,4,8,11-tetraacetic acid (TETA), terpyridine-bis(methyleneamine) tetraacetic acid (TMT), 1,4,7,10-tetraazacyclotridecan-N,N′,N″,N″′-tetraacetic acid (TRITA), and triethylenetetraaminehexaacetic acid (TTHA), N,N′-bis[(6-carboxy-2-pyridil)methyl]-4,13-diaza-18-crown-6 (H₂macropa), 4-amino-4-{2-[(3-hydroxy-1,6-dimethyl-4-oxo-1,4-dihydro-pyridin-2-ylmethyl)-carbamoyl]-ethyl} heptanedioic acid bis-[(3-hydroxy-1,6-dimethyl-4-oxo-1,4-dihydro-pyridin-2-ylmethyl)-amide] (THP), 1,4,7-triazacyclononane-1,4,7-tris[methylene(2-carboxyethyl)phosphinic acid (TRAP), and 2-(4,7,10-tris(2-amino-2-oxoethyl)-1,4,7,10-tetraazacyclododecan-1-yl)acetic acid (DO3AM), 1,4,7,10-tetraazacyclododecane-1,4,7,10-tetrakis[methylene(2-carboxyethylphosphinic acid)] (DOTPI), and S-2-(4-isothiocyanatobenzyl)-1,4,7,10-tetraazacyclododecane tetraacetic acid. The chelating group optionally contains a chelated radioactive or non-radioactive cation.

Using these preferred chelating agents to provide the chelating group in the compound of formula (I), the bond X^{2A} can be formed using a functional group contained in the chelating agent. For example, if —R^{CH} is a residue of a chelating agent which contains a carboxy group, an ester bond or an amide bond X^{2A} can be conveniently provided by reacting the carboxy group, or an activated version thereof, with a suitable reaction partner, e.g. a hydroxy group or an amino group. Preferably, —R^{CH} is a residue of a chelating agent which contains a carboxy group, X^{2A} is an amide bond, and the amide bond X^{2A} is formed using the carboxy group contained in the chelating agent.

More preferably, the chelating agent is selected from DOTA, DOTAGA and TRAP, and still more preferably from DOTA and DOTAGA.

In line with the above, it is particularly preferred that —X^{2A}—R^{CH} is a group of the formula (XCH-1) or (XCH-2)

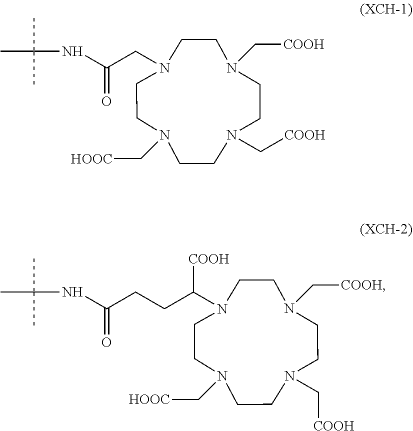

and is attached to the remainder of the compound via the bond marked by the dashed line. The chelating group optionally contains a chelated radioactive or non-radioactive cation.

The chelating group R^{CH} optionally contains a chelated radioactive or non-radioactive cation. The cation may be a metal cation or a non-metallic cation. Preferably, the cation is a radioactive cation. It is also preferred that the cation is a metal cation. Thus, radioactive metal cations are particularly preferred.

Exemplary cations which may be contained as chelated cations in the chelating group are selected from the cations of ⁴³Sc, ⁴⁴Sc, ⁴⁷Sc, ⁵¹Cr, ^{52m}Mn ⁵⁸Co, ⁵²Fe, ⁵⁶Ni, ⁵⁷Ni, ⁶²Cu, ⁶⁴Cu, ⁶⁷Cu, ⁶⁶Ga, ⁶⁸Ga, ⁶⁷Ga, ⁸⁹Zr, ⁹⁰Y, ⁸⁶Y, ⁹⁴mTc, ⁹⁹mTc, ⁹⁷Ru, ¹⁰⁵Rh, ¹⁰⁹Pd, ¹¹¹Ag, ^{110m}In, ¹¹¹In, ^{113m}In, ^{114m}In, ^{117m}Sn, ¹²¹Sn, ¹²⁷Te, ¹⁴²Pr, ¹⁴³Pr, ¹⁴⁷Nd, ¹⁴⁹Gd, ¹⁴⁹Pm, ¹⁵¹Pm, ¹⁴⁹Tb, ¹⁵²Tb, ¹⁵⁵Tb, ¹⁵³Sm, ¹⁵⁶Eu, ¹⁵⁷Gd, ¹⁶¹Tb, ¹⁶⁴Tb, ¹⁶¹Ho, ¹⁶⁶Ho, ¹⁵⁷Dy, ¹⁶⁵Dy, ¹⁶⁶Dy, ¹⁶⁰Er, ¹⁶⁵Er, ¹⁶⁹Er, ¹⁷¹Er, ¹⁶⁶Yb, ¹⁶⁹Yb, ¹⁷⁵Yb, ¹⁶⁷Tm, ¹⁷²Tm, ^{nat}Lu, ¹⁷⁷Lu, ¹⁸⁶Re, ¹⁸⁸Re, ¹⁸⁸W, ¹⁹¹Pt, ^{195m}Pt, ¹⁹⁴Ir, ¹⁹⁷Hg, ¹⁹⁸Au, ¹⁹⁹Au, ²¹²Pb, ²⁰³Pb, ²¹¹At, ²¹²Bi, ²¹³Bi, ²²³Ra, ²²⁴Ra, ²²⁵Ac, and ²²⁷Th, or a cationic molecule comprising ¹⁸F, such as 18F-[AIF]²⁺.

Preferred cations which may be contained as chelated cations in the chelating groups are Ga cations or Lu cations, e.g. ⁶⁸Ga or ¹⁷⁷Lu.

A particularly preferred example of a cation which may be contained as chelated cation in the chelating group is a cation of ¹⁷⁷Lu.

The divalent linking group L¹ preferably comprises two or more subunits which are bonded to each other to form a chain of subunits between X^{1A} and X^{1B}. The one or more bonds between the subunits in this chain of subunits are selected, independently for each occurrence if more than one bond is present, from an amide bond, an ether bond, a thioether bond, an ester bond, a thioester bond, a urea bond, a thiourea bond and an amine bond. As will be understood by the skilled person, the number of bonds depends on the number of subunits, i.e. if two subunits are present, only one bond is present between the subunits. As indicated by the reference to a chain of subunits, the subunits are typically arranged as a linear sequence of subunits extending from X^{1A} to X^{1B}. Preferably, the one or more bonds between the subunits in the chain of units are independently selected for each occurrence from an ether bond, an ester bond and an amide bond, and are more preferably an amide bond. For example, these subunits are provided by hydrocarbon groups which may be substituted by one or more substituents, e.g. substituents selected from —OH, —OCH₃, —COOH, —COOCH₃, —NH₂, —CONH₂, —NHC(O)NH₂, and —NHC(NH)NH₂.

Preferably L¹ comprises 2 to 20 subunits, more preferably 2 to 15 subunits, still more preferably 2 to 12 subunits.

Preferably, L¹ comprises 6 to 40 carbon atoms.

As for the type of subunits, it is preferred that the subunits are independently selected from an alkanediyl unit, a cycloalkanediyl unit, a phenylene unit, an alkanediyl-cycloalkanediyl unit, an alkanediyl-cycloalkanediyl-alkanediyl unit, an alkanediyl-phenylene unit, and an alkanediyl-phenylene-alkanediyl unit, and that the chain of subunits formed from these preferred subunits is optionally substituted by one or more substituents independently selected from —OH, —OCH₃, —COOH, —COOCH₃, —NH₂, —CONH₂, —NHC(O)NH₂, —NHC(NH)NH₂, and aryl. It is more preferred that the chain of subunits formed from these preferred subunits carries no substituent, or is substituted by one, two or three substituents independently selected from —OH, —OCH₃, —COOH, —COOCH₃, —NH₂, —CONH₂, —NHC(O)NH₂ and —NHC(NH)NH₂ and zero or one aryl substituent. An aryl group which may be present as a substituent is, for example, a phenyl or a naphthyl group.

As will be understood by the skilled person, an alkanediyl unit, a cycloalkanediyl unit, and a phenylene unit are divalent units. Moreover, combinations of two or three divalent groups which are directly bound to each other by carbon-carbon bonds, e.g. in an alkanediyl-cycloalkanediyl unit, an alkanediyl-cycloalkanediyl-alkanediyl unit, an alkanediyl-phenylene unit, and an alkanediyl-phenylene-alkanediyl unit, likewise provide divalent units, which are suitable to form a chain of units in line with the above. As will be appreciated, such a combination of two or three divalent groups bound to each other via carbon-carbon bonds, e.g. in an alkanediyl-cycloalkanediyl unit, an alkanediyl-cycloalkanediyl-alkanediyl unit, an alkanediyl-phenylene unit, and an alkanediyl-phenylene-alkanediyl unit, provides a single subunit since no bond selected from an amide bond, an ether bond, a thioether bond, an ester bond, a thioester bond, a urea bond, a thiourea bond and an amine bond is contained in the unit.

An alkanediyl unit of these preferred subunits preferably comprises 1 to 8 carbon atoms. A cycloalkanediyl unit of these preferred subunits preferably comprises 3 to 6 carbon atoms, more preferably 6 carbon atoms. An alkanediyl-cycloalkanediyl unit of these preferred subunits preferably comprises 4 to 10 carbon atoms, more preferably 7 or 8 carbon atoms. An alkanediyl-cycloalkanediyl-alkanediyl unit of these preferred subunits preferably comprises 5 to 10 carbon atoms, more preferably 8 to 10 carbon atoms. An alkanediyl-phenylene unit of these preferred subunits preferably comprises 7 to 10 carbon atoms, more preferably 7 or 8 carbon atoms. An alkanediyl-phenylene-alkanediyl unit of these preferred subunits preferably comprises 8 to 10 carbon atoms, more preferably 8 carbon atoms.

It is further preferred that the alkanediyl groups contained in the preferred subunits are linear (i.e. non-branched) alkanediyl groups. Thus, it is further preferred that the subunits are independently selected from an alkanediyl unit, a cycloalkanediyl unit, a phenylene unit, an alkanediyl-cycloalkanediyl unit, an alkanediyl-cycloalkanediyl-alkanediyl unit, an alkanediyl-phenylene unit, and an alkanediyl-phenylene-alkanediyl unit, wherein all of the alkanediyl groups (singly or in combination with phenylene or cycloalkanediyl) are linear alkanediyl groups, and wherein the chain of units is optionally substituted by one or more substituents independently selected from —OH, —OCH₃, —COOH, —COOCH₃, —NH₂, —CONH₂, —NHC(O)NH₂, —NHC(NH)NH₂, aralkyl and aryl. It is more preferred that the chain of subunits formed from these subunits carries no substituent, or is substituted by one, two or three substituents independently selected from —OH, —OCH₃, —COOH, —COOCH₃, —NH₂, —CONH₂, —NHC(O)NH₂ and —NHC(NH)NH₂ and zero or one substituent selected from aralkyl and aryl. An aryl group which may be present as a substituent is, for example, a phenyl or a naphthyl group. An aralkyl group which may be present as a substituent is, for example, a group —CH₂-phenyl or a group —CH₂-naphthyl. The preferred numbers of carbon atoms for the alkanediyl unit, the cycloalkanediyl unit, the alkanediyl-cycloalkanediyl unit, the alkanediyl-cycloalkanediyl-alkanediyl unit, the alkanediyl-phenylene unit, and the alkanediyl-phenylene-alkanediyl unit indicated above continue to apply.

If L¹ comprises a chain of subunits formed by the preferred subunits defined above, it is further preferred that L¹ comprises not more than one subunit selected from the cycloalkanediyl unit, the phenylene unit, the alkanediyl-cycloalkanediyl unit, the alkanediyl-cycloalkanediyl-alkanediyl unit, the alkanediyl-phenylene unit, and the alkanediyl-phenylene-alkanediyl unit.

It is preferred that the chain of subunits formed by the preferred subunits selected from an alkanediyl unit, a cycloalkanediyl unit, a phenylene unit, an alkanediyl-cycloalkanediyl unit, an alkanediyl-cycloalkanediyl-alkanediyl unit, an alkanediyl-phenylene unit, and an alkanediyl-phenylene-alkanediyl unit, comprises 6 to 40, more preferably 6 to 30 carbon atoms, without carbon atoms contained in the optional substituents. As will be understood, this applies also for the further preferred embodiment wherein the alkanediyl groups are linear alkanediyl groups.

In a more preferred embodiment, the group —X^{1A}-L¹-X^{1B}_in formula (I) is a group of any of the formulae (L-1) to (L-6):

*—NH—C(O)—R^{1L}—C(O)—NH—R^{2L}—NH—C(O)— (L-1)

*—C(O)—NH—R^{3L}—NH—C(O)—R^{4L}—C(O)—NH—R^{5L}—NH—C(O)— (L-2)

*—C(O)—NH—R^{6L}—NH—C(O)—R^{7L}—NH—C(O)—R^{8L}—NH—C(O)—R^{9L}—NH—C(O)— (L-3)

*—C(O)—NH—R^{10L}—NH—C(O)—R^{11L}—NH—C(O)— (L-4)

*—C(O)—NH—R^{12L}—NH—C(O)—R^{13L}—C(O)—NH—R^{14L}—NH—C(O)—R^{15L}—NH—C(O)— (L-5)

*—C(O)—NH—R^{16L}—C(O)—NH—R^{17L}—C(O)—NH—R^{18L}—C(O)—NH— (L-6)
wherein each of R^{1L} to R^{18L} is independently an alkanediyl group containing 1 to 8 carbon atoms, preferably a linear (i.e. non-branched) alkanediyl group containing 1 to 8 carbon atoms,
wherein each of R^{1L} to R^{18L} optionally carries one or more substituents independently selected from —OH, —OCH₃, —COOH, —COOCH₃, —NH₂, —CONH₂, —NHC(O)NH₂, and —NHC(NH)NH₂;
and wherein * marks the bond corresponding to the X^{1A} terminal bond of —X^{1A}-L¹-X^{1B}—.

In an alternative more preferred embodiment, the group —X^{1A}-L¹-X^{1B}— in formula (I) is a group of the formula (L-7):

*—NH—C(O)—R^{19L}—NH—C(O)—R^{20L}—NH—C(O)—R^{21L}—NH—C(O)— (L-7)
wherein R^{19L} is an alkanediyl group containing 1 to 8 carbon atoms, preferably a linear alkanediyl group containing 1 to 8 carbon atoms;
R^{20L} is an alkanediyl group containing 1 to 8 carbon atoms, a cycloalkanediyl group containing 3 to 6 carbon atoms or an alkanediyl-cycloalkanediyl group containing 5 to 8 carbon atoms, wherein each of R^{19L} an R^{20L} is optionally substituted by one or more substituents independently selected from —OH, —OCH₃, —COOH, —COOCH₃, —NH₂, —CONH₂, —NHC(O)NH₂, —NHC(NH)NH₂, aryl and aralkyl;
R^{21L} is an alkanediyl group containing 2 to 26 carbon atoms, preferably a linear alkanediyl group containing 2 to 26 carbon atoms, wherein one or more —CH₂— groups may be replaced by —O—;
and wherein * marks the bond corresponding to the X^{1A} terminal bond of —X^{1A}-L¹-X^{1B}—.

An aryl group which may be present as a substituent is, for example, a phenyl or a naphthyl group. An aralkyl group which may be present as a substituent is, for example, a group —CH₂-phenyl or a group —CH₂-naphthyl.

In formula (L-1), the total number of carbon atoms in R^{1L} and R^{2L} is preferably 6 to 16, without carbon atoms contained in optional substituents that may be carried by the alkanediyl groups.

In formula (L-2), the total number of carbon atoms in R^{3L} to R^{5L} is preferably 6 to 16, without carbon atoms contained in optional substituents.

As noted above, the group L¹ including its preferred embodiments may carry one or more substituents. It is generally preferred that the group L¹ carries one or more substituents, and that at least one of these substituents is a substituent selected from —OH, —OCH₃, —COOH, —COOCH₃, —NH₂, —CONH₂, —NHC(O)NH₂, and —NHC(NH)NH₂. Among these, further preference is given to —COOH. Thus, it is more preferred that the group L¹ carries one or more substituents, and that at least one of these substituents is —COOH.

In a still more preferred embodiment, the group —X^{1A}-L¹-X^{1B}— in formula (I) is a group of the formula (L-8) or (L-9):

*—NH—C(O)—R^{22L}—C(O)—NH—R^{23L}—CH(COOH)—NH—C(O)— (L-8)

*—C(O)—NH—CH(COOH)—R^{24L}—NH—C(O)—R^{25L}—C(O)—NH—R^{26L}—CH(COOH)—NH—C(O)— (L-9)
wherein each of R^{22L} to R^{26L} is independently an alkanediyl group containing 1 to 6 carbon atoms, preferably a linear (i.e. non-branched) alkanediyl group containing 1 to 6 carbon atoms, and wherein * marks the bond corresponding to the X^{1A} terminal bond of —X^{1A}-L¹-X^{1B}—.

In formula (L-8), the total number of carbon atoms in R^{22L} and R^{23L} is preferably 6 to 12, more preferably 8 to 10.

In formula (L-9), the total number of carbon atoms in R^{24L}, R^{25L} and R^{26L} is preferably 6 to 12, more preferably 8 to 10.

In formula (I), —X^{2B}-L² is preferably absent, or the group L² is an alkanediyl group, more preferably a linear alkanediyl group The alkanediyl group may be substituted by one or more substituents independently selected from —OH, —OCH₃, —COOH, —COOCH₃, —NH₂ and —NHC(NH)NH₂. Moreover, one or more covalent bonds between carbon atoms in the alkanediyl group may be independently replaced by a bond selected from an amide bond, an ether bond, a thioether bond, an ester bond, a thioester bond, a urea bond and an amine bond, preferably by a bond selected from an ether bond, an ester bond and an amide bond, and more preferably by an amide bond.

The alkanediyl group preferably contains 2 to 12 carbon atoms, more preferably 2 to 8 carbon atoms. The indicated number of carbon atoms does not include carbon atoms contained in optional substituents and in optional amide, ester, thioester, urea or amine bonds.

If L² is present in the compound of formula (I) as an alkanediyl group as defined above, it is preferred that L² carries no substituent, or is substituted by one or two substituents independently selected from —OH, —COOH, and —NH₂. Moreover, it is preferred that zero, one, or two covalent bonds between carbon atoms are independently replaced by a bond selected from an amide bond, an ether bond, a thioether bond, an ester bond, a thioester bond, a urea bond and an amine bond, preferably by a bond selected from an ether bond, an ester bond and an amide bond, and more preferably by an amide bond.

In formula (I), —X^{3B}-L³ is preferably absent, or the group L³ is an alkanediyl group, more preferably a linear alkanediyl group The alkanediyl group may be substituted by one or more substituents independently selected from —OH, —OCH₃, —COOH, —COOCH₃, —NH₂ and —NHC(NH)NH₂. Moreover, one or more covalent bonds between carbon atoms in the alkanediyl group may be independently replaced by a bond selected from an amide bond, an ether bond, a thioether bond, an ester bond, a thioester bond, a urea bond and an amine bond, preferably by a bond selected from an ether bond, an ester bond and an amide bond, and more preferably by an amide bond.

In one preferred embodiment, the alkanediyl group contains 2 to 12 carbon atoms, more preferably 2 to 8 carbon atoms. The indicated number of carbon atoms does not include carbon atoms contained in optional substituents and in optional amide, ester, thioester, urea or amine bonds.

If L³ is present in the compound of formula (I) as an alkanediyl group as defined above, it is preferred that L³ carries no substituent, or is substituted by one or two substituents independently selected from —OH, —COOH, and —NH₂. Moreover, it is preferred that zero, one, or two covalent bonds between carbon atoms are independently replaced by a bond selected from an amide bond, an ether bond, a thioether bond, an ester bond, a thioester bond, a urea bond and an amine bond, preferably by a bond selected from an ether bond, an ester bond and an amide bond, and more preferably by an amide bond.

In the PSMA ligand compound of formula (I), it is particularly preferred that —X^{2B}-L² is absent and —X^{3B}-L³ is absent.

In another preferred embodiment, L³ is an alkanediyl group of the formula —CH₂—. For example, if L³ is a group of formula —CH₂—, —X^{3B}-L³-X^{3A}— can be a group of the formula —NHC(O)—CH₂—N(CH₃)₂⁺—, and R^{3S} can be a benzyl group wherein the bond with X^{3A} is formed by the —CH₂— moiety at the benzyl group, and wherein the Si atom attached as a substituent to the aromatic ring of the benzyl group and the —CH₂— moiety of the benzyl group are in a para-position to each other.

Preferably, the Si-atom and the chelating group are separated by not more than 25 covalent bonds, more preferably not more than 20 covalent bonds and still more preferably not more than 15 covalent bonds.

In line with the above, R^{B} is preferably a group of the formula (B-1):

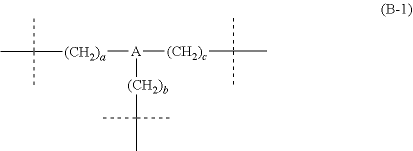

wherein
A is selected from N, CR^{B1} wherein R^{B1} is H or C₁-C₆ alkyl, and a 5 to 7 membered carbocyclic or heterocyclic group; preferably A is selected from N and CH, and more preferably A is CH;
the bond marked by the dashed line at (CH₂)ₐ is formed with X^{1B}, and a is an integer of 0 to 4, preferably 0 or 1, and most preferably 0;
the bond marked by the dashed line at (CH₂)_{b} is formed with X^{3B}, if X^{3B} is present, and otherwise with X^{3A}, and b is an integer of 0 to 4, preferably of 0 to 2, and more preferably 0 or 1;
and the bond marked by the dashed line at (CH₂)_{c} is formed with X^{2B}, if X^{2B} is present, and otherwise with X^{2A}, and c is an integer of 0 to 4, preferably of 0 to 2, and more preferably 0 or 1.

In line with the above, it is further preferred that A is CH, and that a is 0 or 1, b is 0 or 1 and c is 0 or 1, and that a+b+c is 1 or 2.

More preferably, R^{B} in formula (I) is a group of the formula (B-2):

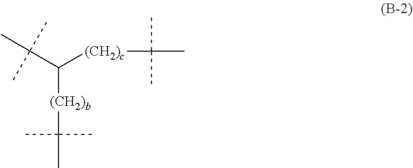

wherein
the bond marked by the dashed line at (CH₂)_{b} is formed with X^{3B}, if present, and otherwise with X^{3A}, and b is an integer of 0 to 4, preferably of 0 to 2, and more preferably 0 or 1;
the bond marked by the dashed line at (CH₂)_{c} is formed with X^{2B}, if present, and otherwise with X^{2A}, and c is an integer of 0 to 4, preferably of 0 to 2, and more preferably 0 or land the remaining bond marked by the dashed line is formed with X^{1B}.

In line with the above, it is preferred that the ligand compound of formula (I) has the following structure (Ia) or the following structure (Ib):

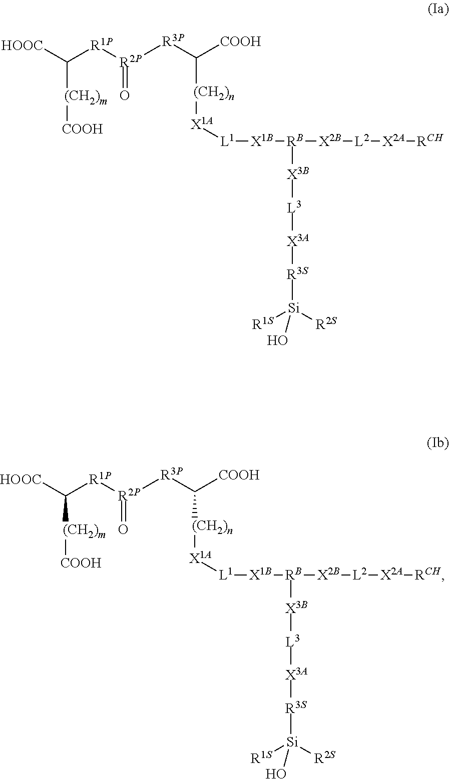

wherein all variables are defined as above, including any preferred variants thereof.

In a further preferred embodiment in line with the above explanations, the ligand compound in accordance with the invention is a compound of formula (II) or a pharmaceutically acceptable salt thereof:

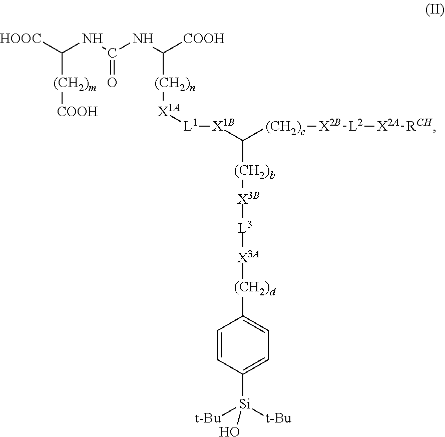

wherein, in formula (II), m, n, b, c, X^{1A}, L¹, X^{1B}, X^{2B}, L², X^{2A}, R^{CH} X^{3A}, L³ and X^{3B} are defined as above, including any preferred variants thereof, and d is 0 or 1.

In a still further preferred embodiment in line with the above explanations, the ligand compound in accordance with the invention is a compound of formula (III) or a pharmaceutically acceptable salt thereof:

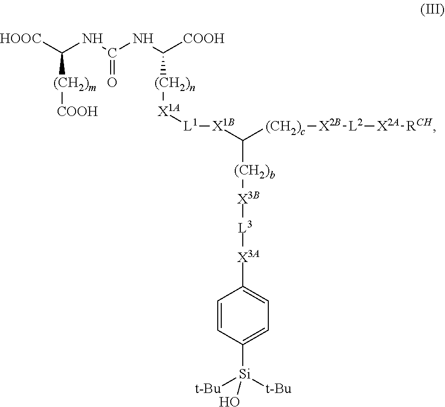

wherein m, n, b, c, X^{1A}, L¹, X^{1B}, X^{2B}, L², X^{2A}, R^{CH} X^{3A}, L³ and X^{3B} are defined as above, including any preferred variants thereof.

In a still more preferred embodiment in line with the above explanations, the ligand compound in accordance with the invention is a compound of formula (IV) or a pharmaceutically acceptable salt thereof

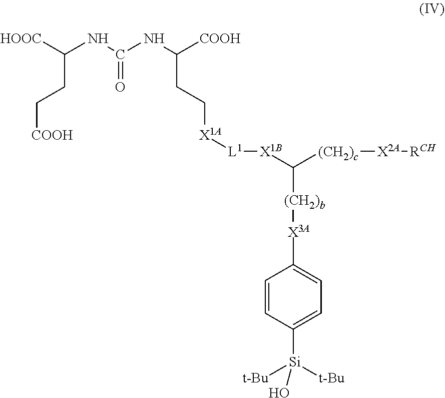

wherein, in formula (IV), b, c, X^{1A}, L¹, X^{1B}, X^{2A}, R^{CH} and X^{3A} are defined as above, including any preferred variants thereof.

More preferably, the PSMA ligand compound of formula (IV) has the following structure (IVa):

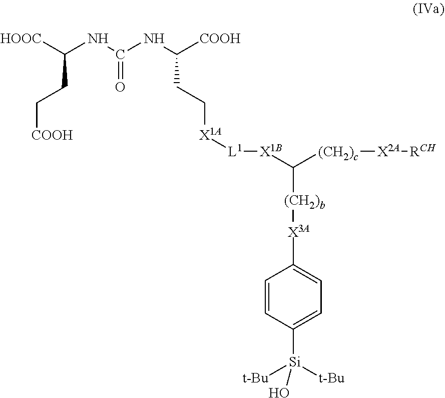

wherein, in formula (IVa), b, c, X^{1A}, L¹, X^{1B}, X^{2A}, R^{CH} and X^{3A} are defined as above, including any preferred variants thereof.

Further preferred ligand compounds in accordance with the invention are represented by any one of the following structures:

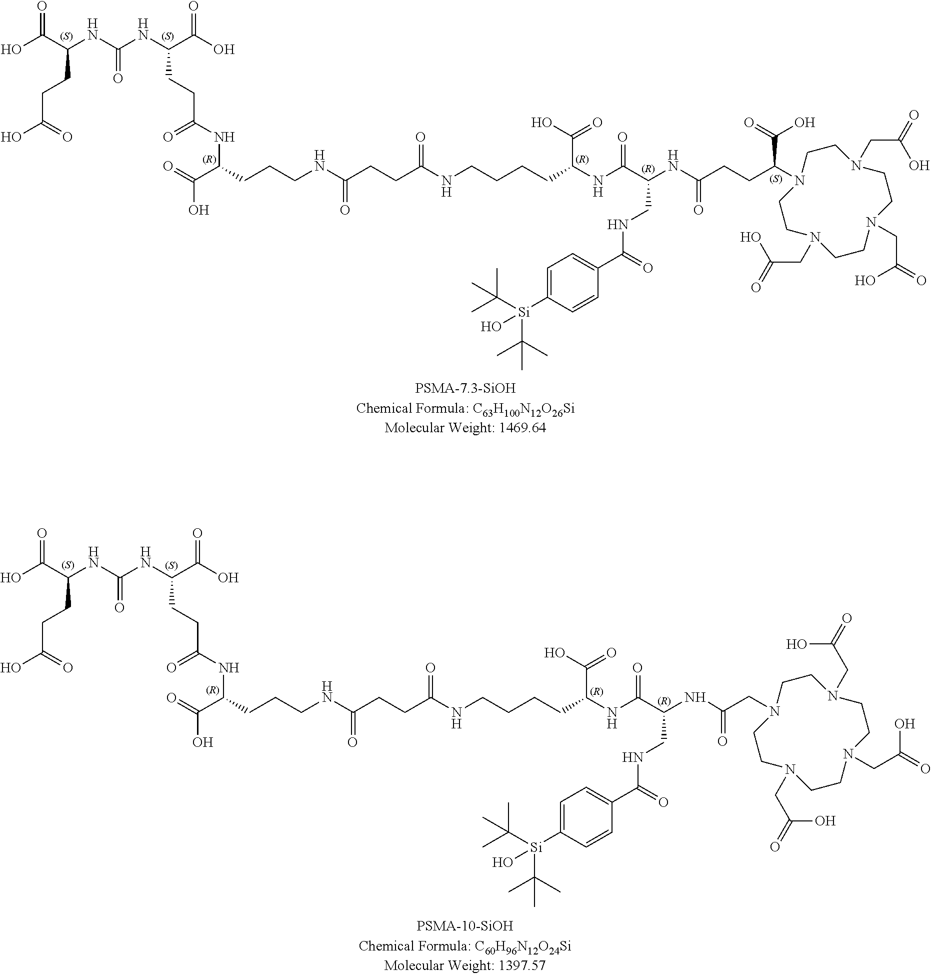

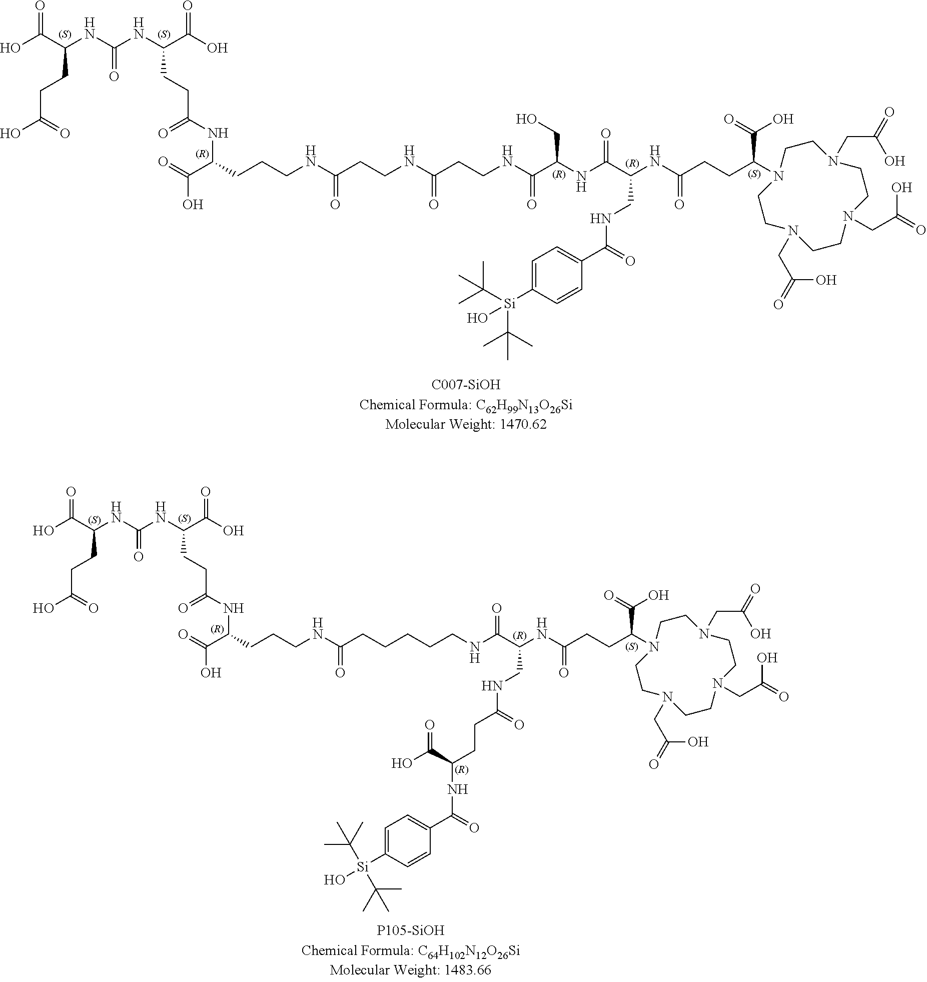

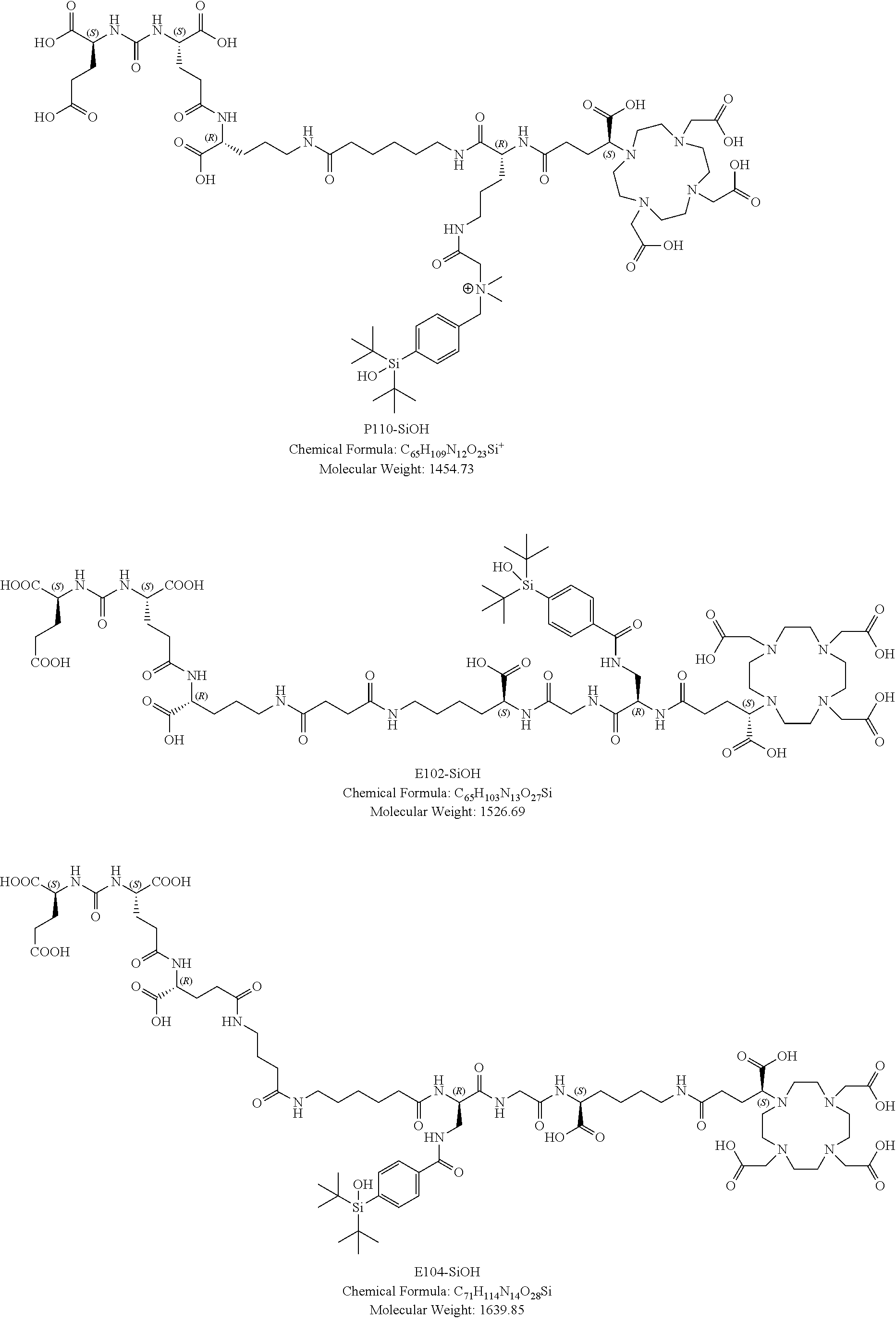

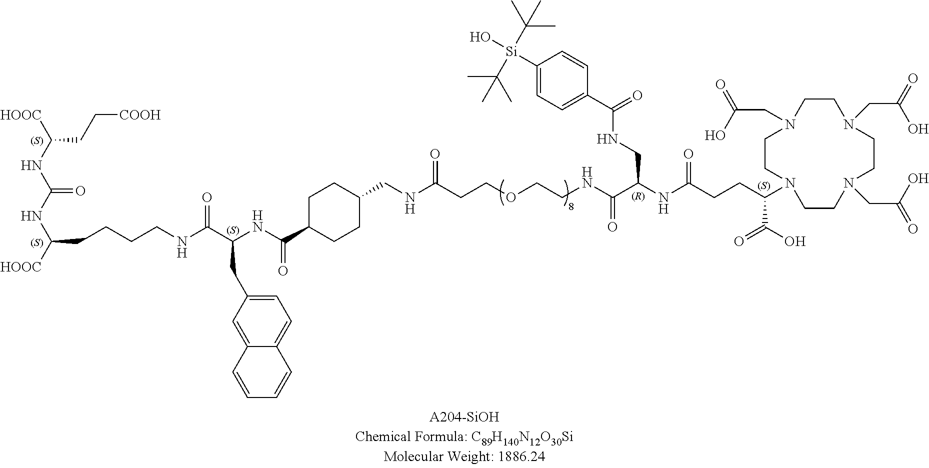

or by a pharmaceutically acceptable salt thereof, wherein the chelating group indicated by the N-heterocyclic moieties in the above formulae optionally contains a chelated radioactive or non-radioactive cation, such as a Ga cation or a Lu cation.

As noted above, the ligand compounds in accordance with the invention may be pharmaceutically acceptable salts. Such salts may be formed, e.g., by protonation of an atom carrying an electron lone pair which is susceptible to protonation, such as a nitrogen atom in an amino group, with an inorganic or organic acid, or as a salt of an organic acid group, e.g. a carboxylic acid group, with a physiologically acceptable cation as they are well known in the art. Exemplary acid addition salts comprise, for example, mineral acid salts such as hydrochloride, hydrobromide, hydroiodide, sulfate salts, nitrate salts, phosphate salts (such as, e.g., phosphate, hydrogenphosphate, or dihydrogenphosphate salts), carbonate salts, hydrogencarbonate salts or perchlorate salts; organic acid salts such as acetate, trifluoroacetate, propionate, butyrate, pentanoate, hexanoate, heptanoate, octanoate, cyclopentanepropionate, undecanoate, lactate, maleate, oxalate, fumarate, tartrate, malate, citrate, nicotinate, benzoate, salicylate or ascorbate salts; sulfonate salts such as methanesulfonate, ethanesulfonate, 2-hydroxyethanesulfonate, benzenesulfonate, p-toluenesulfonate (tosylate), 2-naphthalenesulfonate, 3-phenylsulfonate, or camphorsulfonate salts; and acidic amino acid salts such as aspartate or glutamate salts. Since trifluoroacetic acid is frequently used during the synthesis of peptides, trifluoroacetate salts are typical salts which are provided if a compound comprising a peptide structure is formed. Such trifluoroacetate salts may be converted, e.g., to acetate salts during their workup. Exemplary salts of an organic acid group comprise, for example, alkali metal salts such as sodium or potassium salts; alkaline-earth metal salts such as calcium or magnesium salts; ammonium salts; aliphatic amine salts such as trimethylamine, triethylamine, dicyclohexylamine, ethanolamine, diethanolamine, triethanolamine, procaine salts, meglumine salts, diethanol amine salts or ethylenediamine salts; aralkyl amine salts such as N,N-dibenzylethylenediamine salts, benetamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts or lysine salts.

In terms of lipophilicity/hydrophilicity, the log P value (sometimes also referred to as log D value) is an art-established measure.

The term “lipophilicity” relates to the strength of being dissolved in, or be absorbed in lipid solutions, or being adsorbed at a lipid-like surface or matrix. It denotes a preference for lipids (literal meaning) or for organic or apolar liquids or for liquids, solutions or surfaces with a small dipole moment as compared to water. The term “hydrophobicity” is used with equivalent meaning herein. The adjectives lipophilic and hydrophobic are used with corresponding meaning to the substantives described above.

The mass flux of a molecule at the interface of two immiscible or substantially immiscible solvents is governed by its lipophilicity. The more lipophilic a molecule is, the more soluble it is in the lipophilic organic phase. The partition coefficient of a molecule that is observed between water and n-octanol has been adopted as the standard measure of lipophilicity. The partition coefficient P of a species A is defined as the ratio P=[A]_{n-octanol}/[A]_{water}. A figure commonly reported is the log P value, which is the logarithm of the partition coefficient. In case a molecule is ionizable, a plurality of distinct microspecies (ionized and not ionized forms of the molecule) will in principle be present in both phases. The quantity describing the overall lipophilicity of an ionizable species is the distribution coefficient D, defined as the ratio D=[sum of the concentrations of all microspecies]_{n-octanol}/[sum of the concentrations of all microspecies]_{water}. Analogous to log P, frequently the logarithm of the distribution coefficient, log D, is reported. Often, a buffer system, such as phosphate buffered saline is used as alternative to water in the above described determination of log P.

If the lipophilic character of a substituent on a first molecule is to be assessed and/or to be determined quantitatively, one may assess a second molecule corresponding to that substituent, wherein said second molecule is obtained, for example, by breaking the bond connecting said substituent to the remainder of the first molecule and connecting (the) free valence(s) obtained thereby to hydrogen(s).

Alternatively, the contribution of the substituent to the log P of a molecule may be determined. The contribution πxₓ of a substituent X to the log P of a molecule R—X is defined as πxₓ=log P_{R-X}−log P_{R-H}, wherein R—H is the unsubstituted parent compound.

Values of P and D greater than one as well as log P, log D and πxₓ values greater than zero indicate lipophilic/hydrophobic character, whereas values of P and D smaller than one as well as log P, log D and πxₓ values smaller than zero indicate hydrophilic character of the respective molecules or substituents.

The above described parameters characterizing the lipophilicity of the lipophilic group or the entire molecule according to the invention can be determined by experimental means and/or predicted by computational methods known in the art (see for example Sangster, Octanol-water Partition Coefficients: fundamentals and physical chemistry, John Wiley & Sons, Chichester. (1997)).

In a preferred embodiment, the log P value of the ligand compounds of the invention is between −5 and −1.5. It is particularly preferred that the log P value is between −4.5 and −2.0.

In a further aspect, the present invention provides a therapeutic composition comprising or consisting of one or more ligand compounds in accordance with the invention as disclosed herein above. In a related aspect, the ligand compounds in accordance with the invention are provided for use in a therapeutic method. Preferably, the ligand compound comprises a chelated radioactive cation, such as a ¹⁷⁷Lu cation, and is provided for use in a radiotherapeutic method. Thus, the ligand compound of the invention can be used in a therapeutic method, which method may comprise administering the ligand compound to subject. The subject may be a human or an animal.

In another aspect, the present invention provides a diagnostic composition comprising or consisting of one or more ligand compounds in accordance with the invention as disclosed herein above. In a related aspect, the ligand compounds in accordance with the invention are provided for use in a method of diagnosis, e.g. a method of diagnosis in vivo or in vitro of a disease or disorder. Preferably, the ligand compound comprises a chelated radioactive cation, such as a ⁶⁸Ga cation, and is provided for use in a radiodiagnostic method. Thus, the ligand compound of the invention can be used in a diagnostic method, which method may comprise administering the ligand compound to subject. The subject may be a human or an animal.

As will be understood, the ligand compounds in accordance with the invention for use in medicine, in particular in a therapeutic or diagnostic method generally retain their Si—OH functional moiety during this use. In particular, the use or the method generally does not involve an exchange of the —OH group in the Si—OH functional moiety by a fluorine atom.

The therapeutic or diagnostic composition may further comprise pharmaceutically acceptable carriers, excipients and/or diluents. Examples of suitable pharmaceutical carriers, excipients and/or diluents are well known in the art and include phosphate buffered saline solutions, water, emulsions, such as oil/water emulsions, various types of wetting agents, sterile solutions etc. Compositions comprising such carriers can be formulated by well-known conventional methods. These compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration. It is particularly preferred that said administration is carried out by injection and/or delivery. The compositions may be administered directly to the target site.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Pharmaceutically active matter may be present e.g. in amounts between 0.1 ng and 10 mg/kg body weight per dose; however, doses below or above this exemplary range are envisioned, especially considering the aforementioned factors.

The therapeutic compositions and the therapeutic uses as described herein have to be held distinct from the diagnostic compositions and the diagnostic uses as described herein.

The therapeutic compositions and the therapeutic uses are for a curative purpose; i.e. the treatment or prevention of a disease such as a tumor. The diagnostic compositions and the diagnostic uses are for a diagnostic purpose; i.e. the identification of a disease in a person.

Preferably, the ligand compounds for use in therapy or diagnosis comprise a chelated radioactive cation, more preferably a chelated radioactive metal ion. On the other hand, ligand compounds in accordance with the invention not comprising a chelated radioactive cation can be suitably used, e.g., as precursor compounds for such compounds.

For instance, for diagnosis the chelated radioactive cation is preferably a positron emitter since they are particularly suitable for diagnostics, e.g. via positron emission tomography imaging or single photon emission computerised tomography imaging. Examples of positron emitters are ⁶⁴CU, ⁶⁸Ga, ⁸⁶Y and ^{99m}Tc.

For therapy the chelated radioactive cation is preferably a gamma or beta emitter since they may emit a radiation dose in the target area that weakens or destroys particular targeted cells. Examples of gamma or beta emitters are ¹⁷⁷Lu, ⁸⁹Zr and ¹⁸⁶Re.

As noted above, the present invention provides the ligand compounds of the invention as disclosed herein above for use in medicine.

Preferred uses in medicine are in nuclear medicine such as nuclear diagnostic imaging, also named nuclear molecular imaging, and/or targeted radiotherapy of diseases associated with an overexpression of a target (e.g. a tumor antigen, such as PSMA) on the diseased tissue. The diagnostic imaging is preferably positron emission tomography imaging or single photon emission computerised tomography imaging.

Prostate cancer is not the only cancer to express PSMA. Nonprostate cancers to demonstrate PSMA expression include salivary gland, breast, lung, colorectal, and renal cell carcinoma.

Thus, in a further aspect, the present invention provides a ligand compound of the invention as defined herein above for use in a method of diagnosis in vivo of cancer, preferably prostate cancer.

As used herein, tumor diagnosis comprises detecting the presence or absence of a tumor in a body of a subject and/or staging cancer, i.e. finding out how much tumor is present in the body of a subject, and/or where the tumor is located.

In still a further aspect, the present invention provides a ligand compound of the invention as defined herein above for use in a method of treating cancer, preferably prostate cancer.

In yet a further aspect, the present invention provides a ligand compound of the invention as defined herein above for use in a method for treating or diagnosing neoangiogenisis.

Preferred indications are the detection and/or staging or the treatment of cancer, such as, but not limited high grade gliomas, lung cancer and especially prostate cancer and metastasized prostate cancer, the detection or treatment of metastatic disease in patients with primary prostate cancer of intermediate-risk to high-risk, and the detection or treatment of metastatic sites, even at low serum PSA values in patients with biochemically recurrent prostate cancer. Another preferred indication is the imaging and visualization of neoangiogensis.

Together with the radiohybrid compounds containing a SiFA group, the ligand compounds in accordance with the invention provide a broad selection of structurally related compounds from which single ligand compounds or combinations of ligand compounds can be chosen which are suitable for diverse applications in medicine.

E.g. by relying on the pair of a [18F]rh-ligand compound containing a chelated non-radioactive metal and a Si—OH-ligand compound containing a chelated radioactive metal, the skilled person can provide two molecules with optimized excretion kinetics for the respective diagnostic/therapeutic application, while otherwise the structural variation of the molecules is kept at a minimum.

Exemplary applications include Si—OH ligand compounds containing a chelated radioactive cation such as ¹⁷⁷Lu or ¹¹¹In which can be administered in diagnostic dosages for the radionuclide mediated resection of prostate carcinoma metastases (radioguided surgery). In comparison with the therapeutic use of [¹⁷⁷Lu]rh ligand compounds, this requires faster extraction kinetics, such that tracers administered prior to the operation can be accurately localized during surgery with a manual probe, and the metastases can be identified at a sufficient signal strength.

Moreover, it may be an advantage due to local constraints regarding the production of ¹⁸F if alternatives to [¹⁸F] radiohybrid ligand compounds can be provided which rely e.g. on ⁶⁸Ga as a radioactive marker, and which do not require significant structural changes of the molecular structure compared to the rh ligand. Here, e.g. [Ga-68]-SiOH-ligand compounds provide a suitable alternative since they allow the moderate excretion kinetics of [F-18] rh-ligand compounds to be accelerated and the ⁶⁸Ga to be applied in optimum dosages.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The following examples further illustrate the invention without limiting it to the illustrated embodiments.

### EXAMPLES

### 1. Materials and Methods

### 1.1 General Information

The protected amino acid analogs were purchased from Bachem (Bubendorf, Switzerland), Carbolution Chemicals (St. Ingbert, Germany) or Iris Biotech (Marktredwitz, Germany). The tritylchloride polystyrene (TCP) resin was obtained from Sigma-Aldrich (Steinheim, Germany). Chematech (Dijon, France) delivered the chelators DOTA, DOTA-GA and derivatives thereof. All necessary solvents and other organic reagents were purchased from either, Alfa Aesar (Karlsruhe, Germany), Sigma-Aldrich (Steinheim, Germany), Fluorochem (Hadfield, United Kingdom) or VWR (Darmstadt, Germany). Solid phase synthesis of the peptides was carried out by manual operation using an Intelli-Mixer syringe shaker (Neolab, Heidelberg, Germany). Analytical and preparative reversed-phase high-pressure chromatography (RP-HPLC) was performed using Shimadzu gradient systems (Shimadzu, Neufahrn, Germany), each equipped with a SPD-20A UV/Vis detector (220 nm, 254 nm). A Nucleosil 100 C18 (125×4.6 mm, 5 μm particle size) column (CS Chromatographie Service, Langerwehe, Germany) was used for analytical measurements at a flow rate of 1 mL/min. Both specific gradients and the corresponding retention times t_{R} are cited in the text. Preparative RP-HPLC purification was done with a Multospher 100 RP 18 (250×10 mm, 5 μm particle size) column (CS Chromatographie Service, Langerwehe, Germany) at a constant flow rate of 5 mL/min. Analytical and preparative radio-RP-HPLC was performed using a Nucleosil 100 C18 (125×4.0 mm, 5 μm particle size) column (CS Chromatographie Service, Langerwehe, Germany). Eluents for all HPLC operations were water (solvent A) and acetonitrile (solvent B), both containing 0.1% trifluoroacetic acid. Electrospray ionization-mass spectra for characterization of the substances were acquired on an expression^{L} CMS mass spectrometer (Advion, Harlow, United Kingdom). Radioactivity was detected through connection of the outlet of the UV-photometer to a HERM LB 500 NaI detector (Berthold Technologies, Bad Wildbad, Germany). NMR spectra were recorded on Bruker (Billerica, United States) AVHD-300 or AVHD-400 spectrometers at 300 K. pH values were measured with a SevenEasy pH-meter (Mettler Toledo, Gießen, Germany). Activity quantification was performed using a 2480 WIZARD² automatic gamma counter (PerkinElmer, Waltham, United States). Radio-thin layer chromatography (TLC) was carried out with a Scan-RAM detector (LabLogic Systems, Sheffield, United Kingdom).

### 1.2 Solid Phase Peptide Synthesis

### 1.2.1 TCP-Resin Loading (General Procedure 1 (GP1))

Loading of the tritylchloride polystyrene (TCP) resin with a Fmoc-protected amino acid (AA) was carried out by stirring a solution of the TCP-resin (1.60 mmol/g) and Fmoc-AA-OH (1.5 eq.) in anhydrous DCM with DIPEA (3.8 eq.) at room temperature (rt) for 2 h. Remaining tritylchloride was capped by the addition of methanol (2 mL/g resin) for 15 min. Subsequently the resin was filtered and washed with DCM (2×5 mL/g resin), DMF (2×5 mL/g resin), methanol (5 mL/g resin) and dried in vacuo. Final loading/of Fmoc-AA-OH was determined by the following equation:

$$\begin{matrix} & {m}_{2} = mass of loaded resin [ g ] \\ l [ \frac{m mol}{g} ] = \frac{( {m}_{2} - {m}_{1} ) \times 1000}{( {M}_{W} - {M}_{HCl} ) {m}_{2}} & {m}_{1} = mass of unloaded resin [ g ] \\ & {M}_{W} = molecular weight of AA [ g / mol ] \\ & {M}_{HCl} = molecular weight of HCl [ g / mol ]\end{matrix}$$

### 1.2.2 On-Resin Amide Bond Formation (GP2)

For conjugation of a building block to the resin-bound peptide, a mixture of TBTU with HOBt or HOAt is used for pre-activation of the carboxylic with DIPEA or 2,4,6-trimethylpyridine as a base in DMF (10 mL/g resin). After 5 min at rt, the solution was added to the swollen resin. The exact stoichiometry and reaction time for each conjugation step is given in the respective synthesis protocols. After reaction, the resin was washed with DMF (6×5 mL/g resin).

### 1.2.3 On-Resin Fmoc-Deprotection (GP3)

The resin-bound Fmoc-peptide was treated with 20% piperidine in DMF (v/v, 8 mL/g resin) for 5 min and subsequently for 15 min. Afterwards, the resin was washed thoroughly with DMF (8×5 mL/g resin).

### 1.2.4 On-Resin Dde-Deprotection (GP4)

The Dde-protected peptide was dissolved in a solution of 2% hydrazine monohydrate in DMF (v/v, 5 mL/g resin) and shaken for 20 min (GP4a). In the case of present Fmoc-groups, Dde-deprotection was performed by adding a solution of imidazole (0.92 g/g resin), hydroxylamine hydrochloride (1.26 g/g resin) in NMP (5.0 mL/g resin) and DMF (1.0 mL/g resin) for 3 h at room temperature (GP4b). After deprotection the resin was washed with DMF (8×5 mL/g resin).

1.2.5 Peptide Cleavage from the Resin (GP5)

The fully protected resin-bound peptide was dissolved in a mixture of TFA/TIPS/water (v/v/v; 95/2.5/2.5) and shaken for 30 min. The solution was filtered off and the resin was treated in the same way for another 30 min. Both filtrates were combined, stirred for additional 1-24 h at rt. Product formation was monitored by HPLC. After removing TFA under a stream of nitrogen, the residue was dissolved in a mixture of tert-butanol and water and freeze-dried.

### 1.3 Synthesis of Functional Building Blocks

1.3.1 (S)-5-(tert-Butoxy)-4-(3-((S)-1,5-di-tert-butoxy-1,5-dioxopentan-2-yl)ureido)-5-oxopentanoic acid ((tBuO)EuE(OtBu)₂) (prepared according to WO 2019/020831):

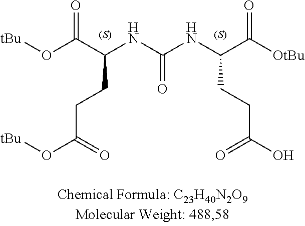

1.3.2 4-(Di-tert-butylfluorosilyl)benzoic acid (SiFA-BA) (prepared according to WO 2019/020831):

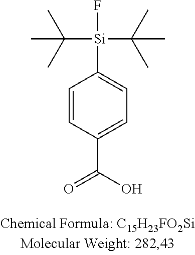 [SMILES: CC(C)(C)[Si](F)(c1ccc(C(=O)O)cc1)C(C)(C)C]

1.3.3 4-(Di-tert-butyl(hydroxy)silyl)benzoic acid (SiOH)

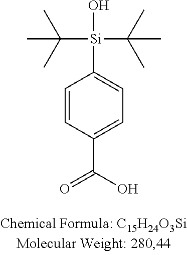 [SMILES: CC(C)(C)[Si](O)(c1ccc(C(=O)O)cc1)C(C)(C)C]

Synthesis of 4-(Di-tert-butylfluorosilyl)benzoic acid was performed according to WO 2019/020831. For hydrolysis, 4-(Di-tert-butylfluorosilyl)benzoic acid (50 mg, 0.177 mmol, 1.0 eq.) was dissolved in a 4:1 mixture (v/v) of DMF and water, whereupon KOH (100 mg, 1.79 mmol, 10 eq.) was added. After stirring the reaction mixture for 4 h at 40° C., the reaction mixture was neutralized by the addition of 1 M aq. HCl. and extracted with Et₂O (5×50 mL). The combined organic fractions were dried, filtered and concentrated in vacuo to give the crude product (95%), which was used without further purification. HPLC (50 to 100% B in 15 min): t_{R}=6.0 min. Calculated monoisotopic mass (C₁₅H₂₄O₃Si): 280.2 found: m/z=281.6 [M+H]⁺.

1.3.4 (4-(Bromomethyl)phenyl)di-tert-butylsilanol

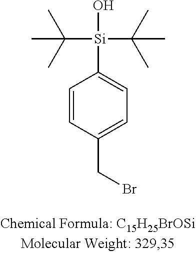 [SMILES: CC(C)(C)[Si](O)(c1ccc(CBr)cc1)C(C)(C)C]

Synthesis of (4-(bromomethyl)phenyl)di-tert-butylsilanol was performed in analogy to a previously published procedure (Kostikov A P, Iovkova L, Chin J, et al., J Fluor Chem. 2011; 132:27-34). HPLC (50 to 100% B in 15 min): t_{R}=11.2 min. Calculated monoisotopic mass (C₁₅H₂₄BrFSi): 328.09 found: m/z=not detectable.

### 1.4 Reference Ligand Compounds

1.4.1 rhPSMA-7.3 (Prepared According to WO 2019/020831):

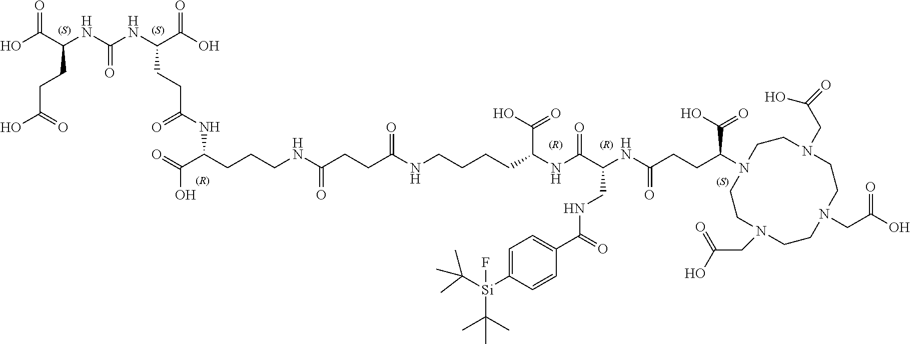

1.4.2 rhPSMA-10 (Prepared According to WO 2019/020831):

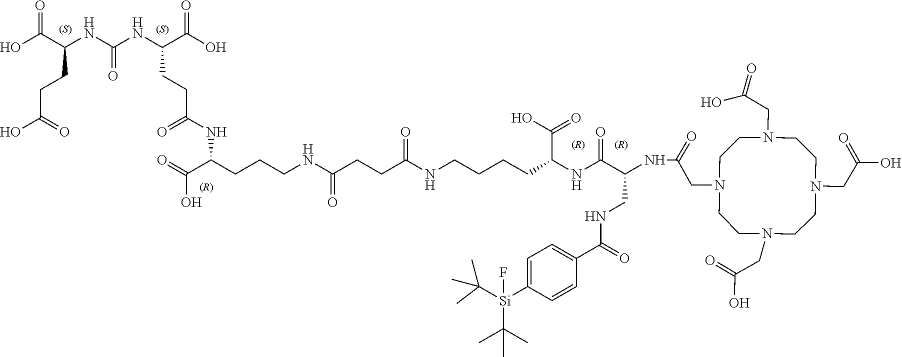

### 1.4.3 PSMA I&T (Weineisen et al.; Journal of Nuclear Medicine 55, 1083-1083 (2014):

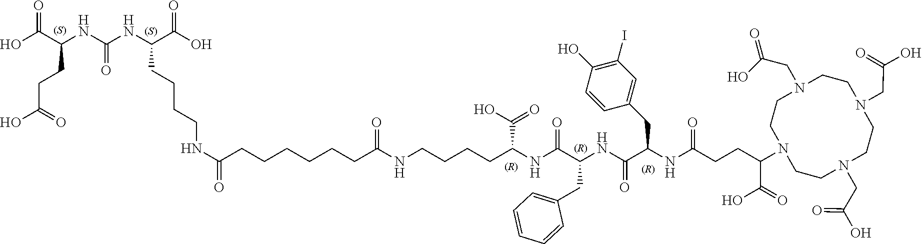

### 1.4.4 PSMA-617 (Benešová et al., Journal of Nuclear Medicine 56, 914-920 (2015)):

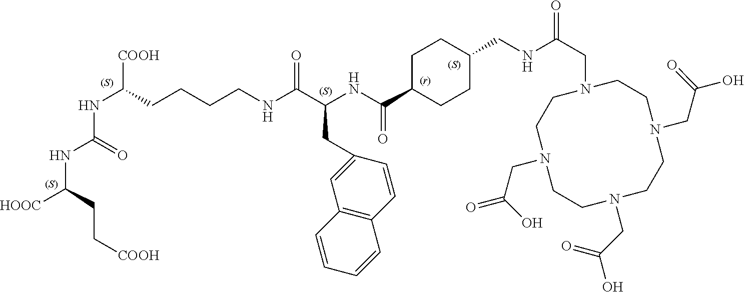

### 1.5 Synthesis of PSMA-SiOH Ligand Compounds

### 1.5.1 PSMA-7.3-SiOH

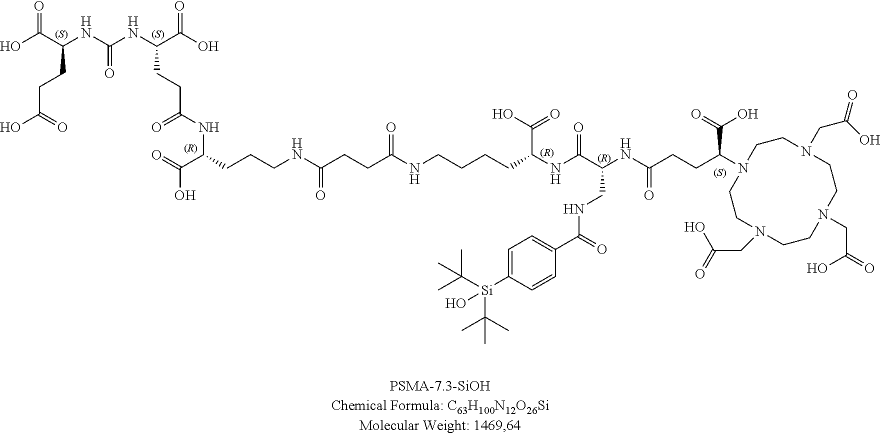

The compound was synthesized, applying the standard Fmoc-SPPS protocol described above, starting from resin bound Fmoc-D-Orn(Dde)-OH. After cleavage of the Fmoc group with piperidine in DMF (GP3), (tBuO)EuE(OtBu)₂ (2.0 eq.) was conjugated with HOAt (2.0 eq.), TBTU (2.0 eq.) and DIPEA (6.0 eq.) in DMF for 4.5 h (GP2). After cleavage of the Dde-group with a mixture hydrazine in DMF (GP4a), a solution of succinic anhydride (7.0 eq.) and DIPEA (7.0 eq.) in DMF was added and left to react for 2.5 h (GP2). Subsequently, the conjugated succinic acid was pre-activated, by adding a mixture of HOAt (2.0 eq.), TBTU (2.0 eq.) and DIPEA (6.0 eq.) in DMF. After 20 min, Fmoc-D-Lys(OtBu)-HCl (2.0 eq.) dissolved in DMF was added and left to react for 2.5 h (GP2). Subsequent cleavage of the Fmoc-group was performed, by adding a mixture piperidine in DMF (GP3). Fmoc-D-Dap(Dde)-OH (2.0 eq.) was pre-activated in a mixture of HOAt (2.0 eq.), TBTU (2.0 eq.) and 2,4,6-trimethylpyridine (6.7 eq.) in DMF and added to the resin-bound peptide for 2.5 h (GP2). Following orthogonal Dde-deprotection was done using imidazole and hydroxylamine hydrochloride dissolved in a mixture of NMP and DMF for 3 h (GP4b). SiOH (1.5 eq.) was reacted with the free amine of the side chain with HOAt (1.5 eq.), TBTU (1.5 eq.) and DIPEA (4.5 eq.), as activation reagents in DMF for 2 h (GP2). After Fmoc-deprotection with piperidine (GP3), (S)-DOTA-GA(tBu)₄ (2.0 eq.) was conjugated with HOAt (2.0 eq.), TBTU (2.0 eq.) and 2,4,6-trimethylpyridine (6.7 eq.) in DMF for 2.5 h (GP2). Cleavage from the resin with simultaneous deprotection of acid labile protecting groups was performed in TFA for 6 h (GP5). After HPLC-based purification PSMA-7.3-SiOH was obtained as a colorless solid (35%). HPLC (10 to 70% B in 15 min): t_{R}=8.6 min. Calculated monoisotopic mass (C₆₃H₁₀₀N₁₂O₂₆Si): 1468.7; found: m/z=1470.0 [M+H]⁺, 735.4 [M+2H]²⁺.

### 1.5.2 PSMA-10-SiOH

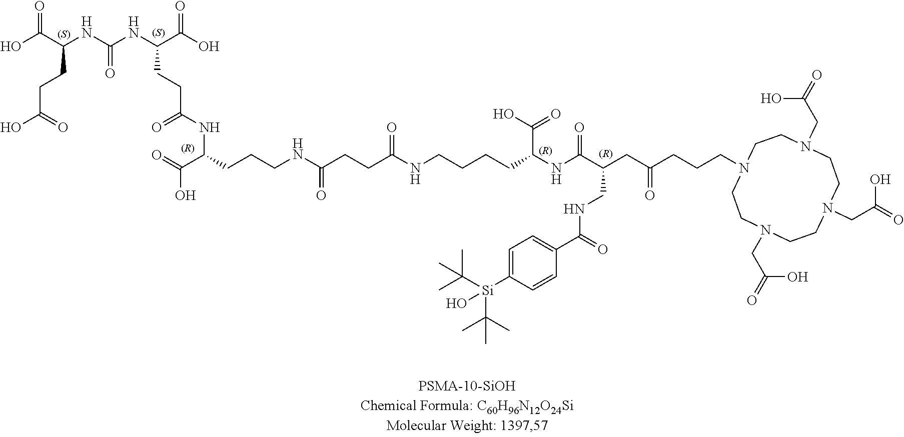

PSMA-10-SiOH was synthesized in analogy to PSMA-7.3-SiOH, by using DOTA instead of DOTA-GA. The tert-butyl protected chelator, DOTA(tBu)₃ was conjugated to the free N-terminus with a mixture of HOAt (2.0 eq.), TBTU (2.0 eq.) and 2,4,6-trimethylpyridine (6.7 eq.) for 2 h in DMF (GP2). Cleavage from the resin and deprotection of acid labile protecting groups was performed in TFA for 6 h (GP5). After HPLC-based purification, PSMA-10-SiOH (34%) was obtained as a colorless solid. HPLC (10 to 70% B in 15 min): t_{R}=8.1 min. Calculated monoisotopic mass (C₆₀H₉₆FN₁₂O₂₄Si): 1396.4; found: m/z=1398.0 [M+H]⁺, 699.5 [M+2H]²⁺.

### 1.5.3 C007-SiOH

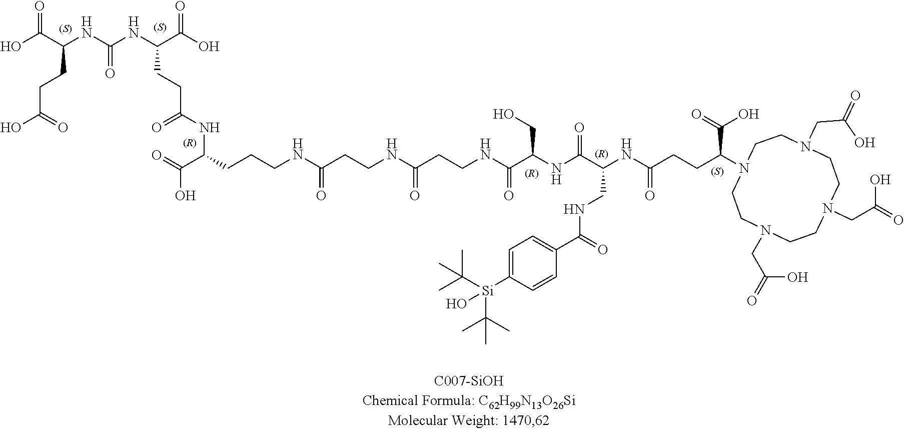

C007-SiOH was synthesized applying the standard Fmoc-SPPS protocol described above, starting from resin bound Fmoc-D-Orn(Dde)-OH. After cleavage of the Fmoc group with piperidine in DMF (GP3), (tBuO)EuE(OtBu)₂ (2.0 eq.) was conjugated with HOAt (2.0 eq.), TBTU (2.0 eq.) and DIPEA (6.0 eq.) in DMF for 4.5 h (GP2) and the Dde-group was subsequently cleaved with a mixture of hydrazine in DMF (GP4a). Fmoc-β-Ala-OH (2.0 eq.), Fmoc-β-Ala-OH (2.0 eq.) and Fmoc-D-Ser(tBu)-OH (2.0 eq.) were then conjugated to the resin-bound peptide. Each coupling was performed for 2.0 h (GP2) after pre-activating the respective amino acid with HOAt (2.0 eq.), TBTU (2.0 eq.) and DIPEA (6.0 eq.) in DMF, each coupling was followed by Fmoc-removal with piperidine (GP3). Fmoc-D-Dap(Dde)-OH (2.0 eq.) was pre-activated in a mixture of HOAt (2.0 eq.), TBTU (2.0 eq.) and 2,4,6-trimethylpyridine (6.7 eq.) in DMF and added to the resin-bound peptide for 2.5 h (GP2). Following orthogonal Dde-deprotection was done using imidazole and hydroxylamine hydrochloride dissolved in a mixture of NMP and DMF for 3 h (GP4b). SiOH (1.5 eq.) was reacted with the free amine of the side chain with HOAt (1.5 eq.), TBTU (1.5 eq.) and DIPEA (4.5 eq.), as activation reagents in DMF for 2 h (GP2). After Fmoc-deprotection with piperidine (GP3), (S)-DOTA-GA(tBu)₄ (2.0 eq.) was conjugated with HOAt (2.0 eq.), TBTU (2.0 eq.) and 2,4,6-trimethylpyridine (6.7 eq.) in DMF for 2.5 h (GP2). Cleavage from the resin with simultaneous deprotection of acid labile protecting groups was performed in TFA for 23 h (GP5). After HPLC-based purification C007-SiOH was obtained as a colorless solid (7%). HPLC (10 to 70% B in 15 min): t_{R}=8.6 min. Calculated monoisotopic mass (C₆₂H₉₉N₁₃O₂₆Si): 1469.7; found: m/z=1470.4 [M+H]⁺, 736.0 [M+2H]²⁺.

### 1.5.4 P105-SiOH

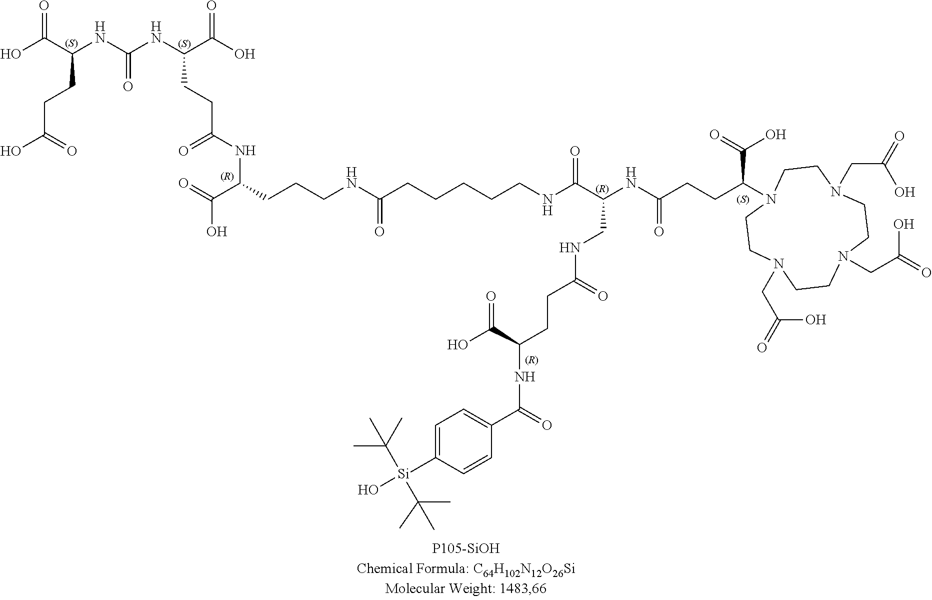

P105-SiOH was synthesized, applying the standard Fmoc-SPPS protocol described above, starting from resin bound Fmoc-D-Orn(Dde)-OH. After cleavage of the Fmoc group with piperidine in DMF (GP3), (tBuO)EuE(OtBu)₂ (2.0 eq.) was conjugated with HOAt (2.0 eq.), TBTU (2.0 eq.) and DIPEA (6.0 eq.) in DMF for 4.5 h (GP2). After cleavage of the Dde-group with a mixture of hydrazine in DMF (GP4a), a solution of Fmoc-6-Ahx-OH (2.0 eq.) in DMF pre-activated for 5 min in a mixture of HOAt (2.0 eq.), TBTU (2.0 eq.) and DIPEA (6.0 eq.) was added to the resin-bound peptide for 2.5 h. After Fmoc-deprotection with piperidine (GP3), Dde-D-Dap(Fmoc)-OH (2.0 eq.) was conjugated with HOAt (2.0 eq.), TBTU (2.0 eq.) and 2,4,6-trimethylpyridine (6.7 eq.) in DMF for 2.5 h (GP2). Following orthogonal Dde-deprotection was done using imidazole and hydroxylamine hydrochloride dissolved in a mixture of NMP and DMF for 3 h (GP4b). (S)-DOTA-GA(tBu)₄ (2.0 eq.) was conjugated with HOAt (2.0 eq.), TBTU (2.0 eq.) and 2,4,6-trimethylpyridine (6.7 eq.) in DMF for 2.5 h (GP2). After subsequent Fmoc-deprotection with piperidine (GP3), Fmoc-D-Glu-OtBu (2.0 eq.) was conjugated with HOAt (2.0 eq.), TBTU (2.0 eq.) and DIPEA (6.0 eq.) for 2.5 h (GP2). Final Fmoc-deprotection was again done with piperidine (GP3) and SiOH (1.5 eq.) was conjugated with HOAt (1.5 eq.), TBTU (1.5 eq.) and DIPEA (4.5 eq.) for 2.5 h (GP2). P105-SiOH was cleaved from the resin within 18 h with simultaneous deprotection of acid labile protecting groups using TFA (GP5). After HPLC-based purification P105-SiOH was obtained as a colorless solid (16%). HPLC (10 to 70% B in min): t_{R}=9.2 min. Calculated monoisotopic mass (C₆₄H₁₀₂N₁₂O₂₆Si): 1482.7; found: m/z=1483.4 [M+H]⁺, 742.3 [M+2H]²⁺.

### 1.5.5 P110-SiOH

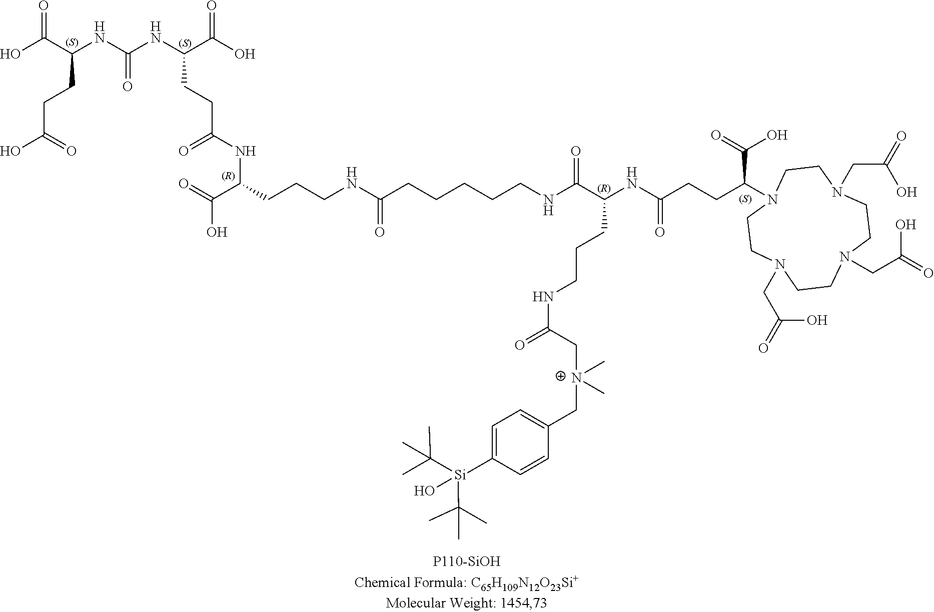

P110-SiOH was synthesized, applying the standard Fmoc-SPPS protocol described above, starting from resin bound Fmoc-D-Orn(Dde)-OH. After cleavage of the Fmoc group with piperidine in DMF (GP3), (tBuO)EuE(OtBu)₂ (2.0 eq.) was conjugated with HOAt (2.0 eq.), TBTU (2.0 eq.) and DIPEA (6.0 eq.) in DMF for 4.5 h (GP2). After cleavage of the Dde-group with a mixture of hydrazine in DMF (GP4a), a solution of Fmoc-6-Ahx-OH (2.0 eq.) in DMF pre-activated for 5 min in a mixture of HOAt (2.0 eq.), TBTU (2.0 eq.) and DIPEA (6.0 eq.) was added to the resin-bound peptide for 2.5 h. After Fmoc-deprotection with piperidine (GP3), Fmoc-D-Orn(Dde)-OH (2.0 eq.) was conjugated with HOAt (2.0 eq.), TBTU (2.0 eq.) and DIPEA (6.0 eq.) in DMF for 2.0 h (GP2). Following orthogonal Fmoc-deprotection was done using piperidine (GP3) and (S)-DOTA-GA(tBu)₄ (2.0 eq.) was conjugated with HOAt (2.0 eq.), TBTU (2.0 eq.) and 2,4,6-trimethylpyridine (6.7 eq.) in DMF for 2.5 h (GP2). After subsequent Dde-deprotection using imidazole and hydroxylamine hydrochloride dissolved in a mixture of NMP and DMF for 3 h (GP4b), N,N-Dimethylglycine (2.0 eq.) was conjugated with HOAt (2.0 eq.), TBTU (2.0 eq.) and DIPEA (6.0 eq.) for 2.5 h (GP2). After final coupling of (4-(bromomethyl)phenyl)di-tert-butylsilanol (3.0 eq.) with 2,4,6-trimethylpyridine (6.0 eq.) in DCM for 18.5 h, P110-SiOH was cleaved from the resin within 21.5 h with simultaneous deprotection of acid labile protecting groups using TFA (GP5). After HPLC-based purification P110-SiOH was obtained as a colorless solid (32%). HPLC (10 to 70% B in 15 min): t_{R}=8.9 min. Calculated monoisotopic mass (C₆₅H₁₀₉N₁₂O₂₃Si⁺): 1453.8; found: m/z=1455.3 [M+H]⁺, 728.0 [M+2H]²⁺.

### 1.5.6 E102-SiOH

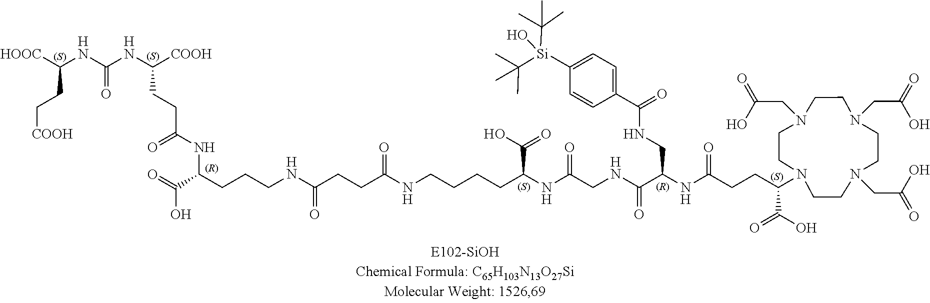

The compound was synthesized, applying the standard Fmoc-SPPS protocol described above, starting from resin bound Fmoc-D-Orn(Dde)-OH. After cleavage of the Fmoc-group with piperidine in DMF (GP3), (tBuO)EuE(OtBu)₂ (2.0 eq.) was conjugated with HOAt (2.0 eq.), TBTU (2.0 eq.) and DIPEA (6.0 eq.) in DMF for 4.5 h (GP2). After cleavage of the Dde-group with a mixture hydrazine in DMF (GP4a), a solution of succinic anhydride (7.0 eq.) and DIPEA (7.0 eq.) in DMF was added and left to react for 2.5 h (GP2). Subsequently, the conjugated succinic acid was pre-activated, by adding a mixture of HOAt (2.0 eq.), TBTU (2.0 eq.) and DIPEA (6.0 eq.) in DMF. After 20 min, Fmoc-L-Lys(OtBu)-HCl (2.0 eq.) dissolved in DMF was added and left to react for 2.5 h (GP2). Subsequent cleavage of the Fmoc-group was performed, by adding a mixture of piperidine in DMF (GP3). Fmoc-Gly-OH (2.0 eq.) was pre-activated in a mixture of HOAt (2.0 eq.), TBTU (2.0 eq.) and DIPEA (6.0 eq.) in DMF and added to the resin-bound peptide for 2 h (GP2). The Fmoc-group was cleaved by adding a mixture of piperidine in DMF (GP3). Fmoc-D-Dap(Dde)-OH (2.0 eq.) was pre-activated in a mixture of HOAt (2.0 eq.), TBTU (2.0 eq.) and 2,4,6-trimethylpyridine (6.7 eq.) in DMF and added to the resin-bound peptide for 2.5 h (GP2). Following orthogonal Dde-deprotection was done using imidazole and hydroxylamine hydrochloride dissolved in a mixture of NMP and DMF for 3 h (GP4b). SiOH (1.5 eq.) was reacted with the free amine of the side chain with HOAt (1.5 eq.), TBTU (1.5 eq.) and DIPEA (4.5 eq.), as activation reagents in DMF for 2 h (GP2). After Fmoc-deprotection with piperidine (GP3), (S)-DOTA-GA(tBu)₄ (2.0 eq.) was conjugated with HOAt (2.0 eq.), TBTU (2.0 eq.) and 2,4,6-trimethylpyridine (6.7 eq.) in DMF for 2.5 h (GP2). Cleavage from the resin with simultaneous deprotection of acid labile protecting groups was performed in TFA for 6 h (GP5). After HPLC-based purification the fluoride-containing ligand E102-SiOH was obtained as a colorless solid (19%). HPLC (10 to 90% B in 15 min): t_{R}=7.4 min. Calculated monoisotopic mass (C₆₅H₁₀₃N₁₃O₂₇Si): 1525.7; found: m/z=1526.9 [M+H]⁺, 763.9 [M+2H]²⁺.

### 1.5.7 E104-SiOH

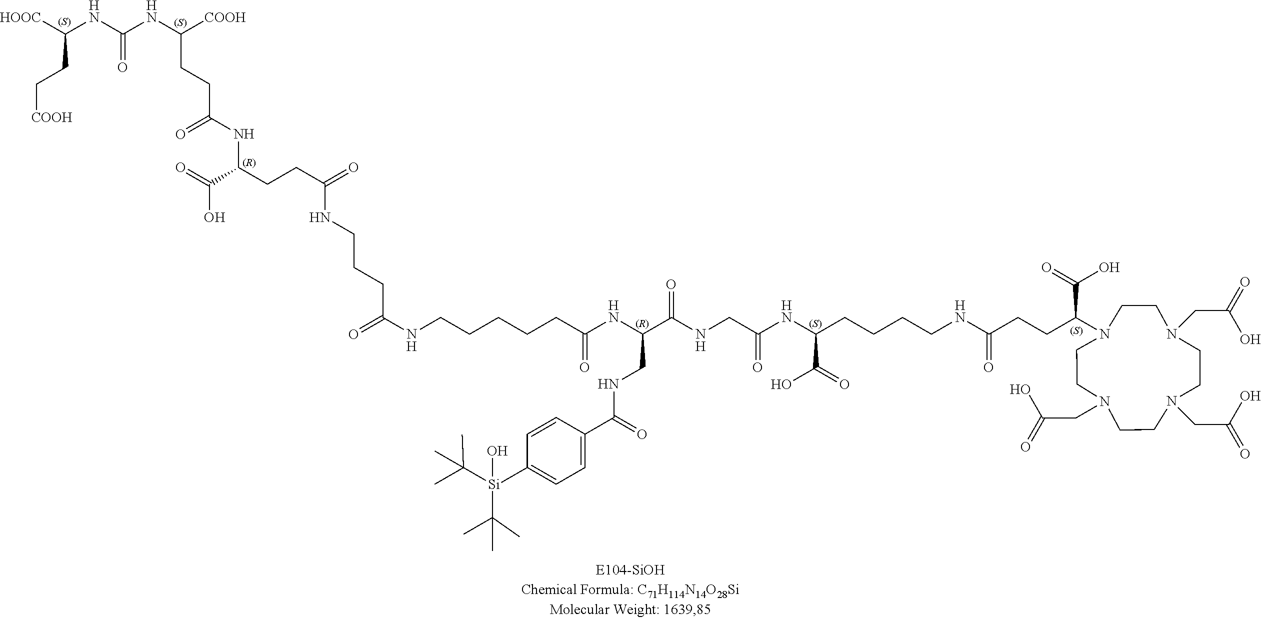

The compound was synthesized, applying the standard Fmoc-SPPS protocol described above, starting from resin bound Fmoc-L-Lys(Dde)-OH. The Dde-group was cleaved by adding imidazole and hydroxylamine hydrochloride dissolved in a mixture of NMP and DMF for 3 h in DMF (GP4b). (S)-DOTA-GA(tBu)₄ (2.0 eq.) was conjugated with HOAt (2.0 eq.), TBTU (2.0 eq.) and 2,4,6-trimethylpyridine (6.7 eq.) in DMF for 2.5 h (GP2). After removal of the Fmoc-group (GP3), Fmoc-Gly-OH (2.0 eq.) was coupled by pre-activation in a mixture of HOAt (2.0 eq.), TBTU (2.0 eq.) and DIPEA (6.0 eq.) in DMF and addition to the resin-bound peptide for 2 h (GP2). The Fmoc-group was cleaved by adding a mixture of piperidine in DMF (GP3). Fmoc-D-Dap(Dde)-OH (2.0 eq.) was pre-activated in a mixture of HOAt (2.0 eq.), TBTU (2.0 eq.) and 2,4,6-trimethylpyridine (6.7 eq.) in DMF and added to the resin-bound peptide for 2.5 h (GP2). Following orthogonal Dde-deprotection was done using imidazole and hydroxylamine hydrochloride dissolved in a mixture of NMP and DMF for 3 h (GP4b). SiOH (1.5 eq.) was reacted with the free amine of the side chain with HOAt (1.5 eq.), TBTU (1.5 eq.) and DIPEA (4.5 eq.), as activation reagents in DMF for 2 h (GP2). After Fmoc-deprotection with piperidine (GP3), Fmoc-Ahx-OH (1.5 eq.) was activated with HOAt (1.5 eq.), TBTU (1.5 eq.) and DIPEA (4.5 eq.) and added to the resin-bound peptide. After Fmoc-removal (GP3), Fmoc-L-Glu-OtBu (1.5 eq.) was pre-activated with HOAt (1.5 eq.), TBTU (1.5 eq.) and DIPEA (4.5 eq.) and coupled to the resin-bound peptide. The Fmoc-group was removed with piperidine in DMF (GP3) and (tBuO)EuE(OtBu)₂ (2.0 eq.) was conjugated with HOAt (2.0 eq.), TBTU (2.0 eq.) and DIPEA (6.0 eq.) in DMF for 4.5 h (GP2). Cleavage from the resin with simultaneous deprotection of acid labile protecting groups was performed in TFA for 6 h (GP5). After HPLC-based purification the fluoride-containing ligand E104-SiOH was obtained as a colorless solid (16%). HPLC (10 to 90% B in 15 min): t_{R}=7.6 min. Calculated monoisotopic mass (C₇₁H₁₁₄N₁₄O₂₈Si): 1638.8; found: m/z=1639.6 [M+H]⁺, 820.8 [M+2H]²⁺.

### 1.5.8 A204-SiOH

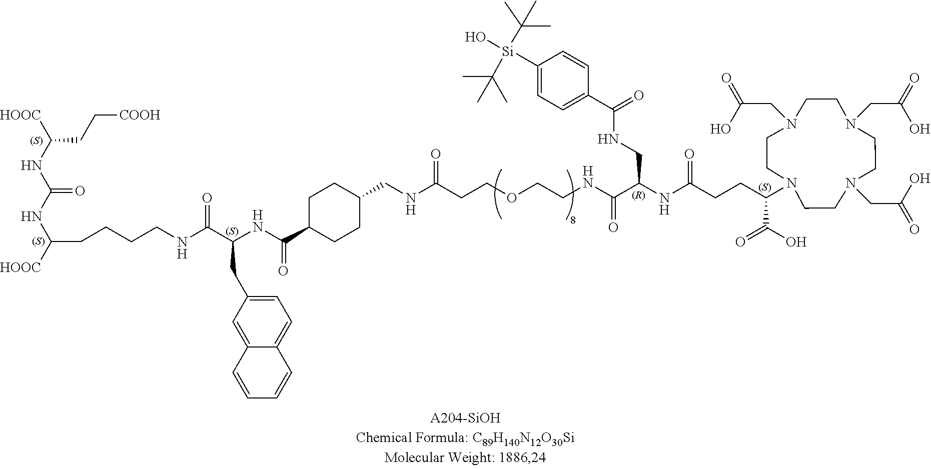

The compound was synthesized, applying the standard Fmoc-SPPS protocol described above, starting from resin bound Fmoc-L-Lys(Dde)-OH. The Fmoc-group was cleaved by adding piperidine in DMF (GP3). Di-tert-butyl (1H-imidazole-1-carbonyl)-L-glutamate (2.0 eq.) was coupled to the resin-bound amino acid, similar to the synthesis of (tBuO)EuE(OtBu)₂, in DCE with TEA (3.0 eq.) at 40° C. for 16 h. Following orthogonal Dde-deprotection was done using imidazole and hydroxylamine hydrochloride dissolved in a mixture of NMP and DMF for 3 h (GP4b). Fmoc-L-2-NaI—OH (2.0 eq.) was coupled after pre-activation with HOAt (2.0 eq.), TBTU (2.0 eq.) and DIPEA (6.0 eq.) in DMF for 2 h. After the removal of the Fmoc-group (GP3), Fmoc-Txa-OH (2.0 eq.) was pre-activated with HOAt (2.0 eq.), TBTU (2.0 eq.) and DIPEA (6.0 eq.) in DMF and coupled to the resin-bound peptide for 2 h. The Fmoc-group was removed according to GP3, Fmoc-NH-PEG₈-COOH (2.0 eq.) was pre-activated with HOAt (2.0 eq.), TBTU (2.0 eq.) and DIPEA (6.0 eq.) in DMF and coupled for 3 h. Fmoc-D-Dap(Dde)-OH (2.0 eq.) was pre-activated in a mixture of HOAt (2.0 eq.), TBTU (2.0 eq.) and 2,4,6-trimethylpyridine (6.7 eq.) in DMF and added to the resin-bound peptide for 2.5 h (GP2). Following orthogonal Dde-deprotection was done using imidazole and hydroxylamine hydrochloride dissolved in a mixture of NMP and DMF for 3 h (GP4b). SiOH (1.5 eq.) was reacted with the free amine of the side chain with HOAt (1.5 eq.), TBTU (1.5 eq.) and DIPEA (4.5 eq.), as activation reagents in DMF for 2 h (GP2). After Fmoc-deprotection with piperidine (GP3), (S)-DOTA-GA(tBu)₄ (2.0 eq.) was conjugated with HOAt (2.0 eq.), TBTU (2.0 eq.) and 2,4,6-trimethylpyridine (6.7 eq.) in DMF for 2.5 h (GP2). Cleavage from the resin with simultaneous deprotection of acid labile protecting groups was performed in TFA for 6 h (GP5). After HPLC-based purification the fluoride-containing ligand A204-SiOH was obtained as a colorless solid (12%). HPLC (10 to 90% B in 15 min): t_{R}=9.0 min. Calculated monoisotopic mass (C₈₉H₁₄₀N₁₂O₃₀Si): 1885.0; found: m/z=1886.6 [M+H]⁺, 944.5 [M+2H]²⁺.

1.6 Synthesis of ^{nat}Lu-PSMA Ligand Compounds

The corresponding ^{nat}Lu-complexes were prepared from a 2 mM aqueous solution of the indicated PSMA ligand compound (1.0 eq.) with a 20 mM solution of LuCl₃ (2.5 eq.), heated to 95° C. for 30 min. After cooling, the ^{nat}Lu-chelate formation was confirmed using HPLC and MS. If required, the complexed compound was purified by RP-HPLC.

^{nat}Lu-PSMA-7.3-SiOH: HPLC (10 to 70% B in 15 min): t_{R}=8.3 min. Calculated monoisotopic mass (C₆₃H₉₇LuN₁₂O₂₆Si): 1640.6; found: m/z=821.8 [M+2H]²⁺.

^{nat}Lu-PSMA-10-SiOH: HPLC (10 to 70% B in 15 min): t_{R}=8.6 min. Calculated monoisotopic mass (C₆₀H₉₃LuN₁₂O₂₄Si): 1568.6; found: m/z=786.2 [M+2H]²⁺.

^{nat}Lu-C007-SiOH: HPLC (10 to 70% B in 15 min): t_{R}=8.3 min. Calculated monoisotopic mass (C₆₂H₉₆LuN₁₃O₂₆Si): 1641.6; found: m/z=821.8 [M+2H]²⁺.

^{nat}Lu-P105-SiOH: HPLC (10 to 70% B in 15 min): t_{R}=8.9 min. Calculated monoisotopic mass (C₆₄H₉₉LuN₁₂O₂₆Si): 1654.6; found: m/z=1655.5 [M+H]⁺, 828.2 [M+2H]²⁺.

^{nat}Lu-P110-SiOH: HPLC (10 to 70% B in 15 min): t_{R}=9.1 min. Calculated monoisotopic mass (C₆₅H₁₀₆LuN₁₂O₂₃Si⁺): 1625.7; found: m/z=1626.2 [M]F, 813.2 [M+H]²⁺.

^{nat}Lu-E102-SiOH: HPLC (10 to 90% B in 15 min): t_{R}=7.2 min. Calculated monoisotopic mass (C₆₅H₁₀₀LuN₁₃O₂₇Si): 1697.6; found: m/z=850.4 [M+2H]²⁺.

^{nat}Lu-E104-SiOH: HPLC (10 to 90% B in 15 min): t_{R}=t.b.d. min. Calculated monoisotopic mass (C₇₁H₁₁₁LuN₁₄O₂₈Si): 1810.7; found: m/z=t.b.d. [M+2H]²⁺.

^{nat}Lu-A204-SiOH: HPLC (10 to 90% B in 15 min): t_{R}=9.0 min. Calculated monoisotopic mass (C₈₉H₁₃₇LuN₁₂O₃₀Si): 2056.9; found: m/z=1030.3 [M+2H]²⁺.

### 1.7 ¹⁷⁷Lu-Labelling of PSMA-SiOH Ligand Compounds

For ¹⁷⁷Lu-labelling a previously published procedure was applied with minor modifications (Sosabowski, J. K.; Mather, S. J., Conjugation of DOTA-like chelating agents to peptides and radiolabeling with trivalent metallic isotopes. Nat Protoc. 2006, 1: 972-976). The labelling precursor (1.0 nmol, 10 μL, 1 mM in DMSO) was added to 10 μL of 1.0 M aq. NH₄OAc buffer (pH 5.9). Subsequently, 20 to 110 MBq ¹⁷⁷LuCl₃ (Specific Activity (S_{A})>3000 GBq/mg, 740 MBq/mL, 0.04 M HCl, ITG, Garching, Germany) were added and the mixture was filled up to 100 μL with water (Tracepur®, Merck, Darmstadt, Germany). The reaction mixture was heated for 30 min at 95° C. and the radiochemical purity was determined using radio-HPLC and radio-TLC (Silica gel 60 RP-18 F₂₅₄s, 3:2 mixture (v/v) of MeCN in H₂O, supplemented with 10% of 2 M NaOAc solution and 1% of TFA).

### 1.8 ¹²⁵I-Labelling

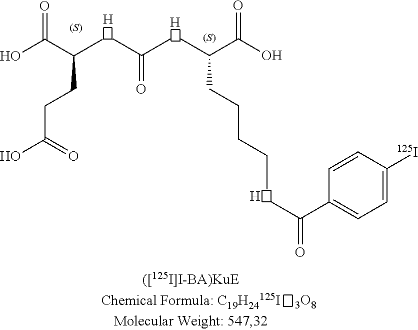

[¹²⁵I]NaI as a basic solution (74 TBq/mmol, 3.1 GBq/mL, 40 mM NaOH) was purchased from Hartmann Analytic (Braunschweig, Germany). The reference ligand for in vitro studies ([¹²⁵I]I-BA)KuE was prepared according to a previously published procedure (Weineisen, M.; Simecek, J.; Schottelius, M.; Schwaiger, M.; Wester, H. J., Synthesis and preclinical evaluation of DOTAGA-conjugated PSMA ligands for functional imaging and endoradiotherapy of prostate cancer. EJNMMI Res 2014, 4; 63; Vaidyanathan, G.; Zalutsky, M. R., Preparation of N-succinimidyl 3-[*I]iodobenzoate: an agent for the indirect radioiodination of proteins. Nat Protoc. 2006, 1, 707-713). Briefly, approximately 0.1 mg of the stannylated precursor SnBu₃-BA-(OtBu)KuE(OtBu)₂ (Vaidyanathan, G. et al, loc. cit.) was dissolved in a mixture of 20 μL peracetic acid, 5.0 μL [¹²⁵I]NaI (40 mM in NaOH, 15±5 MBq, 74 TBq/mmol), 20 μL MeCN and 10 μL acetic acid. The reaction solution was incubated for 10 min at rt, and loaded on a Sep-Pak C18 Plus Short cartridge (360 mg, 55-105 μm, Waters), which was initially preconditioned with 10 mL MeOH followed by 10 mL water). After purging with 10 mL of water, the cartridge was eluted with 2.0 mL of a 1:1 mixture (v/v) of EtOH and MeCN. The eluate was evaporated to dryness under a gentle nitrogen stream at 70° C. and treated with 500 μL TFA for 30 min. After removing TFA in a stream of nitrogen, the crude product was purified by RP-HPLC, yielding ([¹²⁵I]I-BA)KuE (5±2 MBq). HPLC (20% to 40% B in 20 min): t_{R}=13.0 min.

### 1.9 In Vitro-Experiments

### 1.9.1 Cell Culture

PSMA-positive LNCAP cells (300265; Cell Lines Service, Eppelheim, Germany) were cultivated in Dulbecco modified Eagle medium/Nutrition Mixture F-12 with Glutamax (1:1) (DMEM-F12, Biochrom, Berlin, Germany) supplemented with fetal bovine serum (10%, FBS Zellkultur, Berlin, Germany) and kept at 37° C. in a humidified CO₂ atmosphere (5%). A mixture of trypsin and EDTA (0.05%, 0.02%) in PBS (Biochrom) was used in order to harvest cells. Cells were counted with a Neubauer hemocytometer (Paul Marienfeld, Lauda-Königshofen, Germany).

### 1.9.2 Affinity Determinations (IC₅₀)

For PSMA affinity (IC₅₀) determinations, the respective ligand was diluted (serial dilution 10⁻⁴ to 10⁻¹⁰) in Hank's balanced salt solution (HBSS, Biochrom). In the case of metal-complexed ligands, the crude reaction mixture was diluted analogously, without further purification. Cells were harvested 24±2 hours prior to the experiment and seeded in 24-well plates (1.5×10⁵ cells in 1 mL/well). After removal of the culture medium, the cells were carefully washed with 500 μL of HBSS, supplemented with 1% bovine serum albumin (BSA, Biowest, Nuaillé, France) and left 15 min on ice for equilibration in 200 μL HBSS (1% BSA). Next, 25 μL per well of solutions, containing either HBSS (1% BSA, control) or the respective ligand in increasing concentration (10⁻¹-10⁻⁴ M in HBSS) were added with subsequent addition of 25 μL of [¹²⁵I]-BA-KuE (2.0 nM) in HBSS (1% BSA). After incubation on ice for 60 min, the experiment was terminated by removal of the medium and consecutive rinsing with 200 μL of HBSS (1% BSA). The media of both steps were combined in one fraction and represent the amount of free radioligand. Afterwards, the cells were lysed with 250 μL of 1 M aqueous NaOH for at least 10 min. After a washing step (250 μL of 1 M NaOH), both fractions, representing the amount of bound ligand, were united. Quantification of all collected fractions was accomplished in a γ-counter. PSMA-affinity determinations were carried out at least three times per ligand.

### 1.9.3 Internalization Studies

For internalization studies, LNCaP cells were harvested 24±2 hours before the experiment and seeded in poly-L-lysine coated 24-well plates (1.25×105 cells in 1 mL/well, Greiner Bio-One, Kremsmünster, Austria). After removal of the culture medium, the cells were washed once with 500 μL DMEM-F12 (5% BSA) and left to equilibrate for at least 15 min at 37° C. in 200 μL DMEM-F12 (5% BSA). Each well was treated with either 25 μL of either DMEM-F12 (5% BSA, control) or 25 μL of a 100 μM PMPA (2-(Phosphonomethyl)-pentandioic acid, Tocris Bioscience, Bristol, UK) solution in PBS, for blockade. Next, 25 μL of the radioactive-labelled PSMA inhibitor (10.0 nM in PBS) was added and the cells were incubated at 37° C. for 60 min. The experiment was terminated by placing the 24-well plate on ice for 3 min and consecutive removal of the medium. Each well was carefully washed with 250 μL of ice-cold HBSS. Both fractions from the first steps, representing the amount of free radioligand, were combined. Removal of surface bound activity was accomplished by incubation of the cells with 250 μL of ice-cold PMPA (10 μM in PBS) solution for 5 min and rinsed again with another 250 μL of ice-cold PBS. The internalized activity was determined by incubation of the cells in 250 μL 1 M aqueous NaOH for at least 10 min. The obtained fractions were combined with those of the subsequent wash step with 250 μL 1 M aqueous NaOH. Each experiment (control and blockade) was performed in triplicate. Free, surface bound and internalized activity was quantified in a γ-counter. All internalization studies were accompanied by external reference studies, using ([¹²⁵I]l-BA)KuE (c=0.2 nM), which were performed analogously. Data were corrected for non-specific binding and normalized to the specific-internalization observed for the radioiodinated reference compound.

### 1.9.4 Octanol-Water Partition Coefficient

Approximately 1 MBq of the labelled tracer was dissolved in 1 mL of a 1:1 mixture (v/v) of PBS (pH 7.4) and n-octanol in a reaction vial (1.5 mL). After vigorous mixing of the suspension for 3 minutes at room temperature, the vial was centrifuged at 15000 g for 3 minutes (Biofuge 15, Heraeus Sepatech, Osterode, Germany) and 100 μL aliquots of both layers were measured in a gamma counter. The experiment was repeated at least six times.

### 1.9.5 Determination of Human Serum Albumin (HSA) Binding by High Performance Affinity Chromatography (HiPAC)

HSA binding of the PSMA-addressing ligands was determined according to a previously published procedure via HPLC (Valko, K.; Nunhuck, S.; Bevan, C.; Abraham, M. H.; Reynolds, D. P., Fast gradient HPLC method to determine compounds binding to human serum albumin. Relationships with octanol/water and immobilized artificial membrane lipophilicity. J Pharm Sci. 2003, 92, 2236-2248). A Chiralpak HSA column (50×3 mm, 5 μm, H13H-2433, Daicel, Tokyo, Japan) was used at a constant flow rate of 0.5 mL/min at rt. Mobile phase A was a freshly prepared 50 mM aqueous solution of NH₄OAc (pH 6.9) and mobile phase B was isopropanol (HPLC grade, VWR). The applied gradient for all experiments was 100% A (0 to 3 min), followed by 80% A (3 to 40 min). Prior to the experiment, the column was calibrated using nine reference substances with a HSA binding, known from literature, in the range of 13 to 99% (Valko, K.; Nunhuck, S.; Bevan, C.; Abraham, M. H.; Reynolds, D. P., J Pharm Sci. 2003, 92, 2236-2248; Yamazaki, K.; Kanaoka, M., Computational prediction of the plasma protein-binding percent of diverse pharmaceutical compounds. J Pharm Sci. 2004, 93, 1480-1494). All substances, including the examined PSMA ligands, were dissolved in a 1:1 mixture (v/v) of isopropanol and a 50 mM aqueous solution of NH₄OAc (pH 6.9) with a final concentration of 0.5 mg/mL. Non-linear regression was established with the OriginPro 2016G software (Northampton, United States).

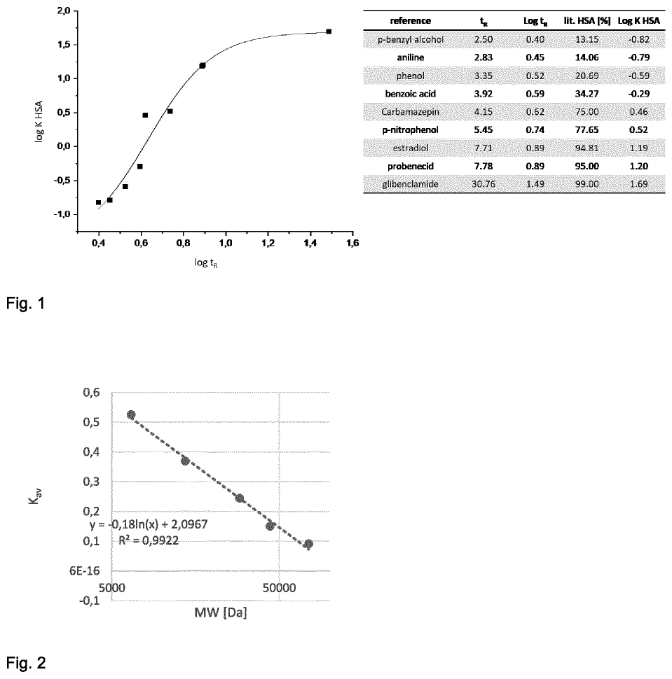

FIG. 1 is an exemplary sigmoidal plot, showing the correlation between human serum albumin (HSA) binding of selected reference substances and retention time (t_{R}). The underlying values of HSA binding also shown in the corresponding table and were obtained from literature (Valko, K.; Nunhuck, S.; Bevan, C.; Abraham, M. H.; Reynolds, D. P., J Pharm Sci. 2003, 92, 2236-2248; Yamazaki, K.; Kanaoka, M., J Pharm Sci. 2004, 93, 1480-1494). Log t_{R}: logarithmic value of experimentally determined retention time. Log K HSA: logarithmic value of HSA binding values.

### 1.9.6 Determination of Human Serum Albumin (HSA) Binding by Radio Inversed Affinity Chromatography (RIAC)

A gel filtration column Superdex 75 Increase 10/300 GL (GE Healthcare, Uppsala, Sweden) was beforehand calibrated following the producer's recommendations with a commercially available gel filtration calibration kit (GE Healthcare, Buckinghamshire, UK) comprising conalbumin (MW: 75 kDa), ovalbumin (44 kDa), carbonic anhydrase (29 kDa), ribonuclease A (13.7 kDa) and aprotinin (6.5 kDa) as reference proteins of known molecular weight. RIAC experiments were conducted using a constant flow rate of 0.8 mL/min at rt. A solution of HSA in PBS at physiological concentration (700 μM) was used as the mobile phase. PSMA ligands were labelled as described with molar activities of 10-20 GBq/μmol. Probes of 1.0 MBq of the radioligand were injected directly from the labelling solution. HSA binding was expressed as an apparent molecular weight MW calculated from the retention time of the radioligand using the determined calibration curve.

FIG. 2 shows a calibration plot of Superdex 75 Increase gel filtration column using a low molecular weight gel filtration calibration kit. The following table summarizes the underlying data. MW: molecular weight. t_{R}: experimentally determined retention time. V: elution volume. Kₐᵥ: partition coefficient.

| reference | MW | t_{R} | V | Kₐᵥ |
|---|---|---|---|---|
| blue dextran | | 10.034 | 8.027 | |
| 2000 | | | | |
| conalbumin | 75000 | 11.859 | 9.487 | 0.0914 |
| ovalbumin | 44000 | 13.034 | 10.427 | 0.1503 |
| carbonic | 29000 | 14.912 | 11.930 | 0.2443 |
| anhydrase | | | | |
| ribonuklease A | 13700 | 17.398 | 13.918 | 0.3688 |
| aprotinin | 6500 | 20.531 | 16.425 | 0.5257 |

The RIAC method is based on the Hummel-Dreyer-method, which displays ligand-protein-binding in a gel filtration chromatographic experiment using protein containing samples and a ligand containing mobile phase (Soltes L. The Hummel-Dreyer method: impact in pharmacology. Biomed Chromatogr. 2004; 18:259-271). RIAC, in an inversed way, comprises ligand samples (radiolabeled PSMA ligands) and a protein (HSA) containing mobile phase. Ligand-specific retention is caused by interaction of the ligand probe and HSA in the mobile phase. Unbound species, due to a molecular weight beneath the molecular cut off of the gel filtration column, are maximally retained while bound species are eluted in accordance to the higher molecular weight of the ligand-HSA-complex (MW_{HSA}: 66.47 kDa). Experiments show a single radiopeak indicating a dynamic process of formation and dissociation of the ligand-HSA-complex throughout the passage through the column bed. Accordingly, the absolute retention time is the result of the ligand's mean time of abidance at HSA which is directly defined by the ligand's binding kinetics and affinity toward HSA. For evaluation, experimentally determined retention times t_{R} are first converted into elution volumes Vₑ by multiplying with the flow rate and thereafter converted into partition coefficients Kₐᵥ following the equation

$${K}_{av} = \frac{{V}_{e} - {V}_{0}}{{V}_{c} - {V}_{0}}$$

where V₀ is the column void volume (8.027 mL) and V_{c} is the geometric column volume (24 mL). Using the equation given by the trend line plot of the column calibration

Kₐᵥ=−0.18 ln(MW)+2.0967

the apparent molecular weight MW is calculated as

$$MW = {e}^{\frac{2.0967 - {K}_{av}}{0.18}}$$

The retention time of HSA determined via UV-detection with PBS as mobile phase and a HSA-containing protein sample was found to be t_{R}(HSA)=11.792 min, the retention time of [¹⁸F]fluoride under standard experimental conditions serving as a non-binding sample was found to be t_{R}([¹⁸F]fluoride)=24.351 min opening a detection window of 70.2 kDa (apparent MW of HSA) to 2.3 kDa (apparent MW of [¹⁸F]fluoride; beneath molecular cut-off of the gel filtration column) defining maximal and minimal HSA-binding of evaluated PSMA-ligands.

### 1.10 In Vivo Experiments

### 1.10.1 General Information

All animal experiments were conducted in accordance with general animal welfare regulations in Germany (German animal protection act, as amended on 18 May 2018, Art. 141 G v. 29.3.2017|626, approval no. 55.2-1-54-2532-71-13) and the institutional guidelines for the care and use of animals. To establish tumor xenografts, LNCaP cells (approx. 10⁷ cells) were suspended in 200 μL of a 1:1 mixture (v/v) of DMEM F-12 and Matrigel (BD Biosciences, Germany), and inoculated subcutaneously onto the right shoulder of 6-8 weeks old CB17-SCID mice (Charles River, Sulzfeld, Germany). Mice were used for experiments when tumors had grown to a diameter of 5-10 mm (3-6 weeks after inoculation).

### 1.10.1 Biodistribution Studies

Approximately 5-10 MBq (0.1-0.2 nmol) of the radioactive-labelled PSMA inhibitors were injected into the tail vein of LNCaP tumor-bearing male CB-17 SCID mice and sacrificed after 24 h post injection. Selected organs were removed, weighted and measured in a γ-counter.

### 2. Results

### 2.1 Affinity

### 2.1.1 Half Maximal Inhibitory Concentration (IC₅₀)

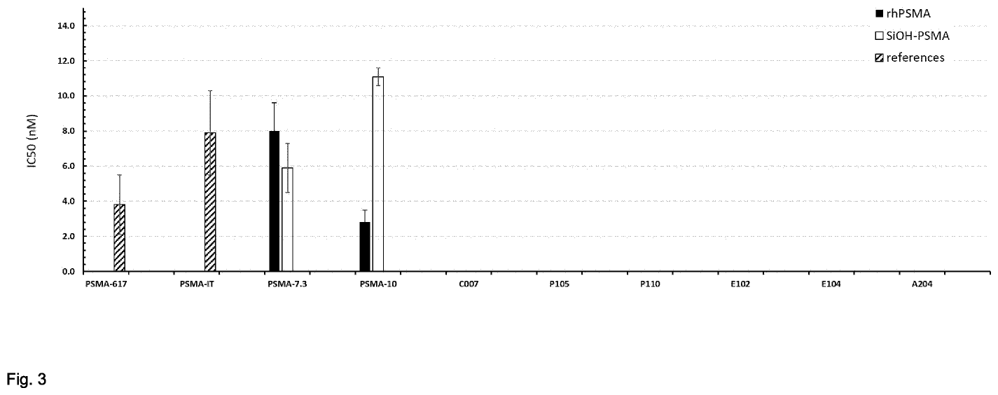

FIG. 3 and the following table shows the binding affinities (IC₅₀ in nM) of novel ligand compounds and references to PSMA. Affinities were determined using LNCaP cells (150000 cells/well) and ([¹²⁵I]I-BA)KuE (c=0.2 nM) as the radioligand (1 h, 4° C., HBSS+1% BSA). Data are expressed as mean±SD (n=3).

| compound | IC₅₀ |
|---|---|
| [^{nat}Lu] - x | PSMA-SiOH |
| 7.3-SiOH | 5.9 ± 1.4 |
| 10-SiOH | 11.1 ± 0.5 |
| C007-SiOH | 7.1 ± 0.9 |
| P105-SiOH | 9.8 ± 0.4 |
| P110-SiOH | 42.2 ± 6.2 |
| E102-SiOH | 8.4 ± 2.4 |
| E104-SiOH | 9.0 ± 2.2 |
| A204-SiOH | 13.5 ± 3.2 |
| PSMA-617 | 3.8 ± 1.7 |
| PSMA I&T | 7.9 ± 2.4 |
| rhPSMA-7.3 | 8.0 ± 1.6 |
| rhPSMA-10 | 2.8 ± 0.7 |

### 2.1.2 Discussion of Results

In the present investigation, the substitution of a fluoride by a hydroxide at the SiFA-benzoyl unit had little impact on the affinity comparing 7.3-SiOH and 10-SiOH with their radiohybride (rh) counterparts, respectively. The determined IC50 values of the majority of the SiOH-ligands were in a one-digit region or only slightly worse. Putting these findings into a context of ongoing clinical trials with the PSMA ligand DCFPyL (18F-DCFPyL Positron Emission Tomography (PET) in Intermediate or High Risk Prostate Cancer, https://clinicaltrials.gov/ct2/show/NCT04727736), exhibiting an IC50 value of 12.3±1.2 nM in the same assay (Robu et al., EJNMMI (2018) 8:30), PSMA affinity in this scope proofs to be sufficient.

### 2.2 Internalization

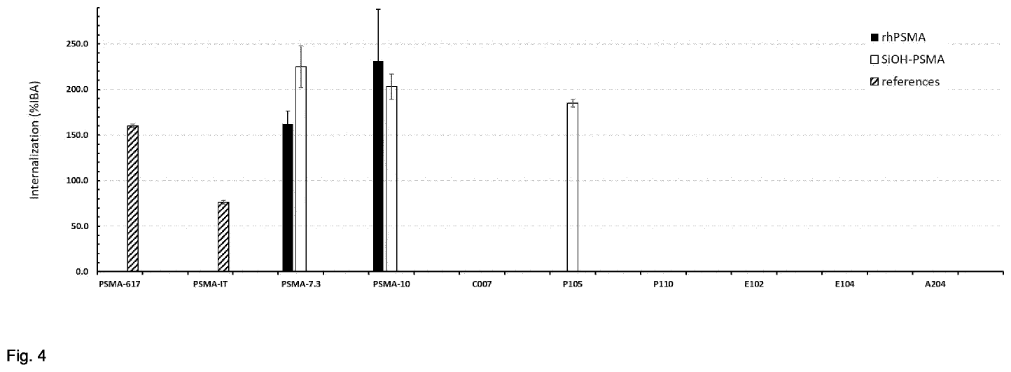

FIG. 4 and the following table shows a summary of the internalized activity (c=1.0 nM) at 1 hour as % of the reference ligand ([¹²⁵I]I-BA)KuE (c=0.2 nM), determined on LNCaP cells (37° C., DMEM F12+5% BSA, 125000 cells/well). Data is corrected for non-specific binding (10 μmol PMPA) and expressed as mean±SD (n=3-6):

| compound | Internalization (% IBA) |
|---|---|
| [¹⁷⁷Lu] - x | PSMA-SiOH |
| 7.3-SiOH | 225 ± 23 |
| 10-SiOH | 203 ± 14 |
| C007-SiOH | 185 ± 15 |
| P105-SiOH | 185 ± 4 |
| P110-SiOH | 46 ± 2 |
| E102-SiOH | 203 ± 15 |
| E104-SiOH | 103 ± 2 |
| A204-SiOH | 85 ± 5 |
| PSMA-617 | 160 ± 2 |
| PSMA I&T | 76 ± 2 |
| rhPSMA-7.3 | 162 ± 14 |
| rhPSMA-10 | 231 ± 57 |

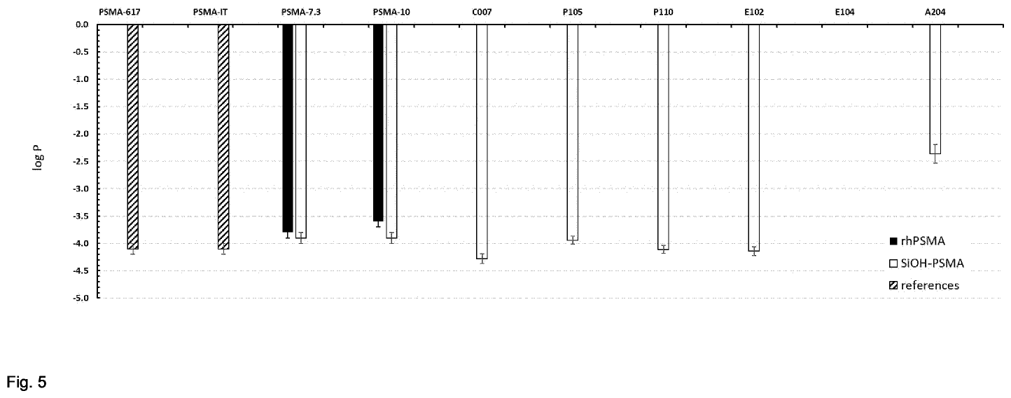

FIG. 5 and the following table summarize log P_{O/W} values of the synthesized radiolabeled ¹⁷⁷Lu-PSMA ligand compounds and ¹⁷⁷Lu-references (n=6).

| compound | Log P_{O/W} |
|---|---|
| [¹⁷⁷Lu] - x | PSMA-SiOH |
| 7.3-SiOH | −3.9 ± 0.1 |
| 10-SiOH | −3.8 ± 0.1 |
| C007-SiOH | −4.28 ± 0.09 |
| P105-SiOH | −3.94 ± 0.07 |
| P110-SiOH | −4.11 ± 0.07 |
| E102-SiOH | −4.14 ± 0.08 |
| E104-SiOH | −3.77 ± 0.16 |
| A204-SiOH | −2.36 ± 0.17 |
| PSMA-617 | −4.1 ± 0.1 |
| PSMA I&T | −4.1 ± 0.1 |
| rhPSMA-7.3 | −3.8 ± 0.1 |
| rhPSMA-10 | −3.6 ± 0.1 |

### 2.4 Binding to Human Serum Albumin (HSA)

### 2.4.1 HSA Binding by High-Performance Affinity Chromatography (HiPAC)

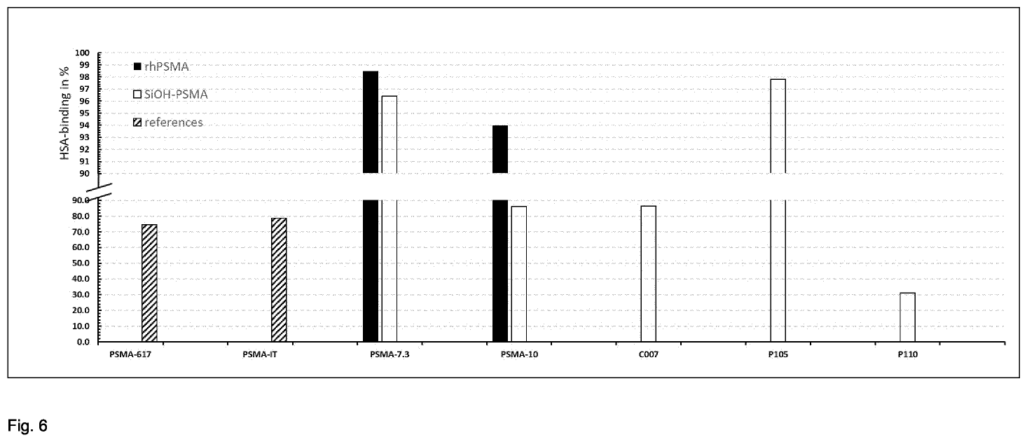

FIG. 6 and the table in 2.4.2 below shows the HSA-binding of lutetium-complexed PSMA-ligand compounds and reference compounds determined by HiPAC on a Chiralpak HSA column (50×3 mm, 5 μm, H13H-2433).

### 2.4.2 HSA Binding by Radio Inversed Affinity Chromatography (RIAC)

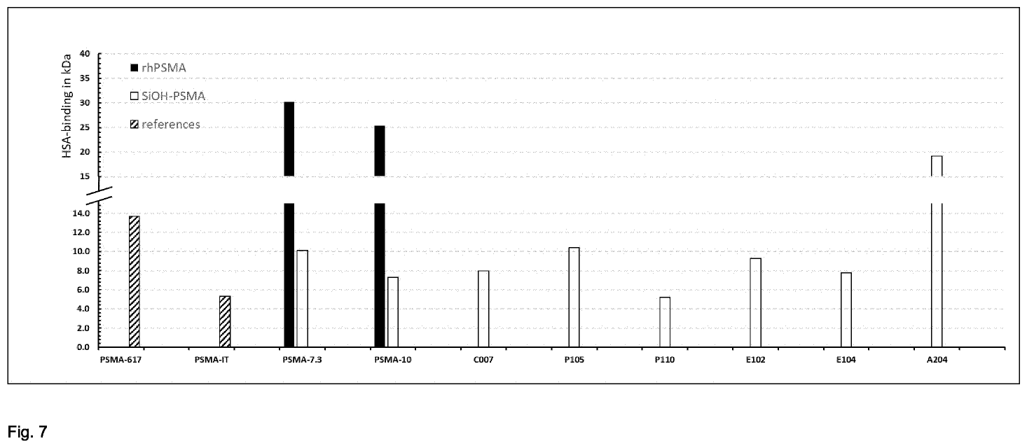

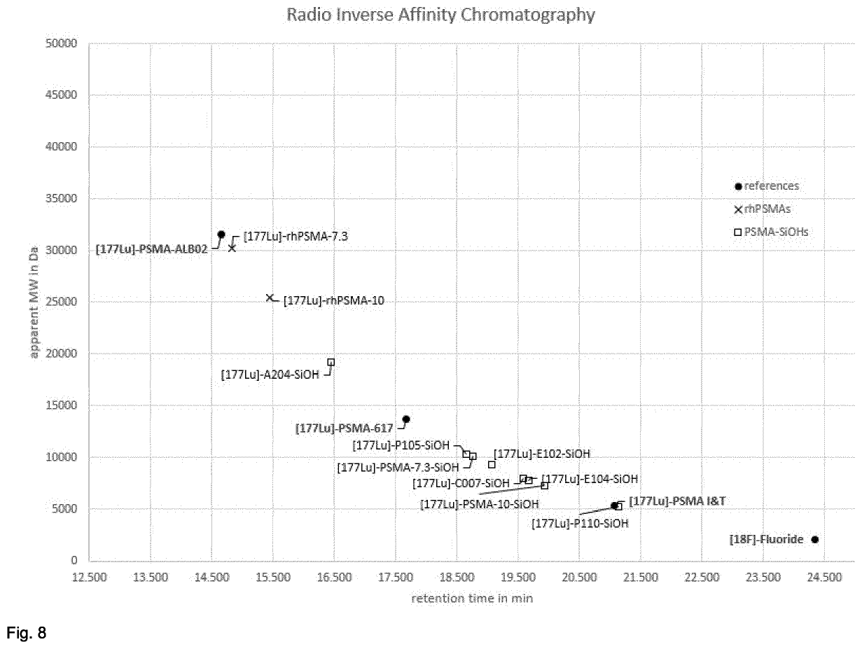

FIG. 7 and the table below show the results HSA-binding of lutetium-complexed PSMA-ligands and reference compounds determined by RIAC on a Superdex 75 Increase 10/300 GL column 700 μM HSA in PBS as solvent using a constant flow rate of 0.8 mL/min (plotted data, extracted from FIG. 8).

FIG. 8 illustrates the determination of HSA binding via radio inverse affinity chromatography (RIAC). Correlation of apparent molecular weight (MW) in Dalton (Da) and retention time of ¹⁷⁷Lu-labeled samples, determined on a Superdex 75 Increase 10/300 GL with 700 μM HSA in PBS as solvent using a constant flow rate of 0.8 mL/min.

| | | HSA-binding |
|---|---|---|
| compound | HSA-binding | RIAC [kDa] |
| [Lu]PSMA | HiPAC [%] | (see FIG. 7) |
| 7.3-SiOH | 96.4 | 10.1 |
| 10-SiOH | 86.0 | 7.3 |
| P105-SiOH | 97.8 | 10.4 |
| P110-SiOH | 31.3 | 5.2 |
| C007-SiOH | 86.3 | 8.0 |
| E102-SiOH | 95.1 | 9.3 |
| E104-SiOH | 85.8 | 7.8 |
| A204-SiOH | 87.3 | 19.2 |
| PSMA I&T | 78.6 | 5.3 |
| PSMA-617 | 74.7 | 13.7 |
| Alb-02 (10) | 97.7 | 31.6 |
| rhPSMA-7.3 | 98.5 | 30.2 |
| rhPSMA-10 | 94.0 | 25.4 |

### 2.4.3 Discussion of Results

The analyzed SiOH-PSMA ligands mostly show high HSA-binding of above 85% when determined by High Performance HSA Affinity Chromatography (HiPAC), indicating stronger interaction with HSA than for PSMA-617 and PSMA-I&T (74.7% and 78.6%, respectively). Ligand P110-SiOH constitutes the only exception (31.3%), which can be attributed to a positive charge within the SiFA/in-group probably hampering the ligands interaction with the basic drug binding sites I and II of HSA (Ghuman, 2005, doi: 10.1016/j.jmb.2005.07.075). Comparison of compounds PSMA-7.3-SiOH and PSMA-10-SiOH with their respective radiohybrid counterparts reveals nearly identical (96.4% vs 98.5%) and slightly decreased (86.0% vs 94.0%) HSA-binding for PSMA-7.3-SiOH and PSMA-10-SiOH, respectively. At first glance, these results seem to reflect the high similarity in structure and lipophilicity of radiohybrid compounds and their respective SiOH-analogs. However, in contrast to these findings, surprising differences in HSA-binding of rhPSMA and SiOH-PSMA ligands were found when measured with Radio Inverse Affinity Chromatography (also named AMSEC for Albumin Mediated Size Exclusion Chromatography). While high apparent Molecular Weights of 30.2 and 25.4 kDa were determined for rhPSMA-7.3 and rhPSMA-10, respectively, MWₐₚₚ obtained for the analog SiOH-PSMA ligands was only 10.1 and 7.3 kDa, respectively. Thus, instead of showing similarity to their radiohybrid analogs, these apparent Molecular Weights are significantly lower. This trend was further corroborated by MWₐₚₚ of approximately 5-10 kDa found for the remaining SiOH-PSMA compounds discussed herein. It becomes apparent, that the HSA-binding capacity of rh/SiOH-PSMA ligands in terms of apparent Molecular Weight is predominantly governed by the differing HSA-binding properties of the SiFA-group and the SiOH-group.

While both methods, HiPAC and RIAC, suggest different strength of HSA-binding for SiOH-PSMA ligands compared to rhPSMA and reference compounds, several inherent features of RIAC might qualify this methodology to predict in vivo albumin-binding more precisely. Firstly, protein-ligand-interactions in RIAC take place in solution and at physiologic pH (pH(PBS)=7.4) and HSA-concentration (700 μM), while HiPAC is based on interaction of ligands with immobilized HSA in an isopropanol containing eluent. Additionally, the output dimension of Molecular Weight [kDa] in RIAC might allow for estimation of implications on plasma half-life and excretion in vivo. In a similar fashion, apparent molecular size of PASylated proteins with expanded hydrodynamic volume was analyzed by Schlapschy et al. (Schlapschy, 2013, doi: 10.1093/protein/gzt023) Apparent molecular sizes determined in SEC experiments exceeded actual molecular sizes of the PASylated proteins and were correlated to significantly increased plasma half-life in vivo (compared to un-PASylated proteins). We therefore concluded that the surprising effect of the SiOH-group on the in vitro HSA-binding of SiOH-PSMA ligands can be exploited to create SiOH-PSMA ligands with more rapid and complete excretion kinetics (decreased residual binding to circulating HSA) compared to analog rhPSMA compounds.

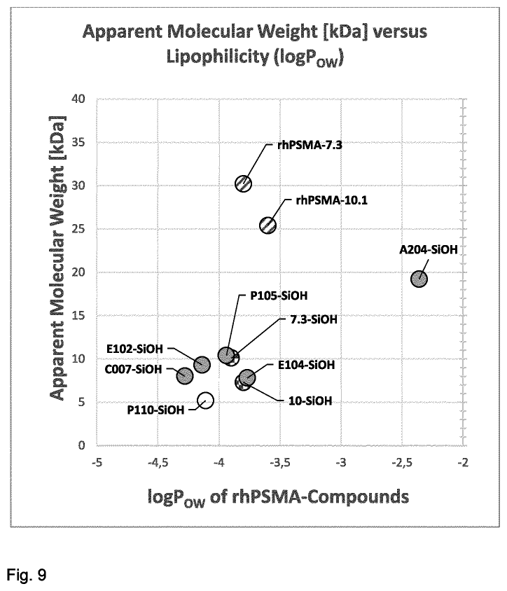

FIG. 9 summarizes the Apparent Molecular Weight [kDa] as measured by Radio Inverse Affinity Chromaotography (RIAC) versus lipophilicity (log P_{OW}=octanol/water partition coefficient) of the Lu-177 labeled SiOH-ligands and the Lu-177 labeled rhPSMA ligands [Lu-177]rhPSMA7-3 and [Lu-177]rhPSMA-10

Comparative biodistribution studies of [¹⁷⁷Lu]-rhPSMA-7.3 vs [¹⁷⁷Lu]-PSMA-7.3-SiOH and [¹⁷⁷Lu]-rhPSMA-10 vs [¹⁷⁷Lu]-PSMA-10-SiOH 24h p.i. confirm the hypothesis of more rapid and complete excretion kinetics for SiOH-PSMA ligands compared to their rhPSMA counterparts

### 2.5 Biodistribution in LNCaP-Tumor-Bearing Mice (24 h Post Injection)

2.5.1 Biodistribution: rhPSMA-7.3 Vs. PSMA-7.3-SiOH

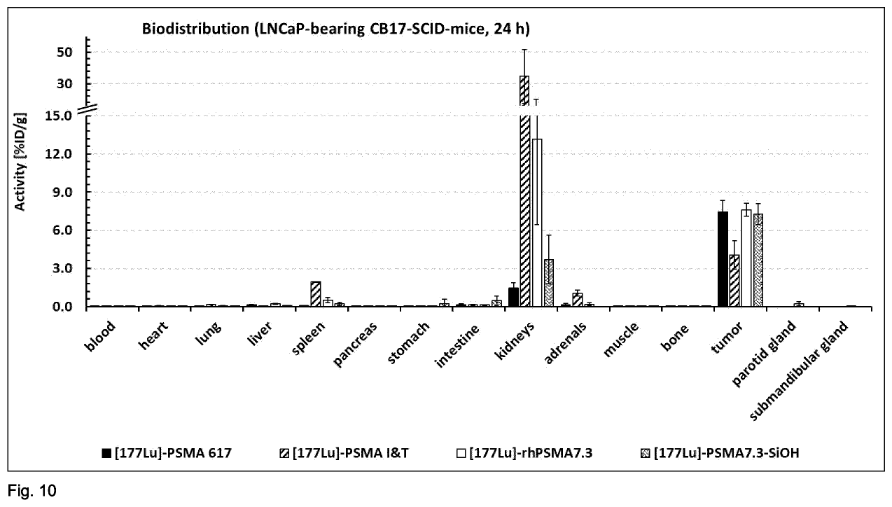

FIG. 10 and the following table show the biodistribution data 24 h p.i. of ¹⁷⁷Lu-labeled compounds in LNCaP tumor-bearing CB17-SCID mice. Data are expressed as mean±SD (n=4-5).

| | [¹⁷⁷Lu] | [¹⁷⁷Lu] | [¹⁷⁷Lu] | [¹⁷⁷Lu] |
|---|---|---|---|---|
| organs | PSMA I&T | PSMA-617 | rh7.3 | 7.3-SiOH |
| blood | 0.012 ± 0.01 | 0.006 ± 0.01 | 0.004 ± 0.002 | 0.002 ± 0.001 |
| heart | 0.05 ± 0.03 | 0.01 ± 0.01 | 0.03 ± 0.01 | 0.01 ± 0.01 |
| lung | 0.16 ± 0.03 | 0.04 ± 0.01 | 0.06 ± 0.02 | 0.03 ± 0.01 |
| liver | 0.05 ± 0.01 | 0.12 ± 0.06 | 0.22 ± 0.03 | 0.08 ± 0.03 |
| spleen | 1.94 ± 1.01 | 0.08 ± 0.01 | 0.51 ± 0.22 | 0.21 ± 0.11 |
| pancreas | 0.05 ± 0.02 | 0.01 ± 0.01 | 0.02 ± 0.01 | 0.01 ± 0.01 |
| stomach | 0.05 ± 0.02 | 0.02 ± 0.01 | 0.05 ± 0.01 | 0.24 ± 0.36 |
| intestine | 0.12 ± 0.06 | 0.12 ± 0.08 | 0.12 ± 0.03 | 0.45 ± 0.39 |
| kidneys | 34.7 ± 17.2 | 1.4 ± 0.4 | 13.2 ± 6.7 | 3.7 ± 1.9 |
| adrenals | 1.06 ± 0.24 | 0.13 ± 0.12 | — | — |
| muscle | 0.01 ± 0.01 | 0.01 ± 0.01 | 0.012 ± 0.004 | 0.006 ± 0.005 |
| bone | 0.01 ± 0.01 | 0.03 ± 0.01 | 0.02 ± 0.01 | 0.01 ± 0.01 |
| tumor | 4.1 ± 1.1 | 7.5 ± 0.9 | 7.6 ± 0.5 | 7.3 ± 0.8 |
| parotis | | | 0.22 ± 0.16 | |
| submandibularis | | | 0.05 ± 0.02 | |

2.5.2 Biodistribution: rhPSMA-10 vs. PSMA-10-SiOH

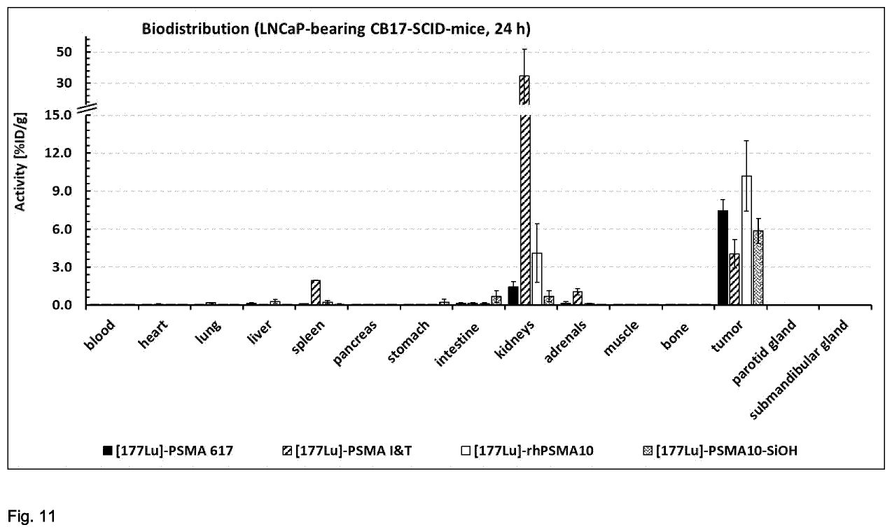

FIG. 11 and the following table show the biodistribution data 24 h p.i. of ¹⁷⁷Lu-labeled compounds in LNCaP tumor-bearing CB17-SCID mice. Data are expressed as mean±SD (n=4-5).

| | [¹⁷⁷Lu] | [¹⁷⁷Lu] | [¹⁷⁷Lu] | [¹⁷⁷Lu] |
|---|---|---|---|---|
| organs | PSMA I&T | PSMA-617 | rh10 | 10-SiOH |
| blood | 0.012 ± 0.01 | 0.006 ± 0.01 | 0.004 ± 0.001 | 0.002 ± 0.002 |
| heart | 0.05 ± 0.03 | 0.01 ± 0.01 | 0.03 ± 0.01 | 0.01 ± 0.01 |
| lung | 0.16 ± 0.03 | 0.04 ± 0.01 | 0.05 ± 0.01 | 0.02 ± 0.01 |
| liver | 0.05 ± 0.01 | 0.12 ± 0.06 | 0.28 ± 0.13 | 0.05 ± 0.01 |
| spleen | 1.94 ± 1.01 | 0.08 ± 0.01 | 0.22 ± 0.11 | 0.07 ± 0.04 |
| pancreas | 0.05 ± 0.02 | 0.01 ± 0.01 | 0.02 ± 0.01 | 0.01 ± 0.01 |
| stomach | 0.05 ± 0.02 | 0.02 ± 0.01 | 0.04 ± 0.01 | 0.21 ± 0.25 |
| intestine | 0.12 ± 0.06 | 0.12 ± 0.08 | 0.11 ± 0.06 | 0.66 ± 0.48 |
| kidneys | 34.7 ± 17.2 | 1.4 ± 0.4 | 4.1 ± 2.3 | 0.69 ± 0.44 |
| adrenals | 1.06 ± 0.24 | 0.13 ± 0.12 | 0.09 ± 0.06 | 0.01 ± 0.01 |
| muscle | 0.01 ± 0.01 | 0.01 ± 0.01 | 0.010 ± 0.003 | 0.005 ± 0.002 |
| bone | 0.01 ± 0.01 | 0.03 ± 0.01 | 0.02 ± 0.01 | 0.02 ± 0.01 |
| tumor | 4.1 ± 1.1 | 7.5 ± 0.9 | 10.2 ± 2.8 | 5.9 ± 1.0 |

### 2.5.3 Biodistribution: C007-SiOH

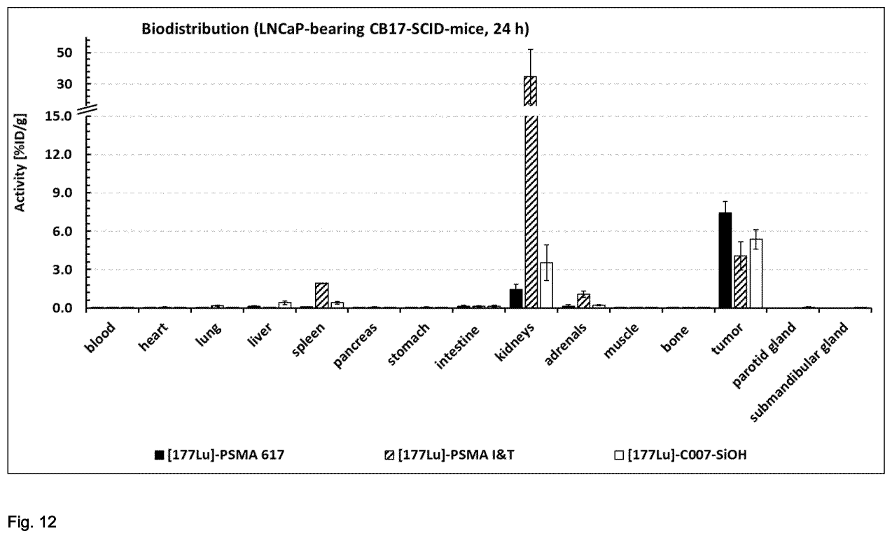

FIG. 12 and the following table show the biodistribution data 24 h p.i. of ¹⁷⁷Lu-labeled compounds in LNCaP tumor-bearing CB17-SCID mice. Data are expressed as mean±SD (n=4-5).

| | [¹⁷⁷Lu] | [¹⁷⁷Lu] | [¹⁷⁷Lu] |
|---|---|---|---|
| organs | PSMA I&T | PSMA-617 | C007-SiOH |
| blood | 0.012 ± 0.01 | 0.006 ± 0.01 | 0.003 ± 0.001 |
| heart | 0.05 ± 0.03 | 0.01 ± 0.01 | 0.02 ± 0.01 |
| lung | 0.16 ± 0.03 | 0.04 ± 0.01 | 0.04 ± 0.01 |
| liver | 0.05 ± 0.01 | 0.12 ± 0.06 | 0.40 ± 0.13 |
| spleen | 1.94 ± 1.01 | 0.08 ± 0.01 | 0.40 ± 0.10 |
| pancreas | 0.05 ± 0.02 | 0.01 ± 0.01 | 0.01 ± 0.01 |
| stomach | 0.05 ± 0.02 | 0.02 ± 0.01 | 0.04 ± 0.01 |
| intestine | 0.12 ± 0.06 | 0.12 ± 0.08 | 0.13 ± 0.08 |
| kidneys | 34.7 ± 17.2 | 1.4 ± 0.4 | 3.5 ± 1.4 |
| adrenals | 1.06 ± 0.24 | 0.13 ± 0.12 | 0.20 ± 0.01 |
| muscle | 0.01 ± 0.01 | 0.01 ± 0.01 | 0.004 ± 0.001 |
| bone | 0.01 ± 0.01 | 0.03 ± 0.01 | 0.02 ± 0.01 |
| tumor | 4.1 ± 1.1 | 7.5 ± 0.9 | 5.4 ± 0.8 |
| parotis | | | 0.06 ± 0.03 |
| submandibularis | | | 0.03 ± 0.01 |

### 2.5.4 Biodistribution P105-SiOH

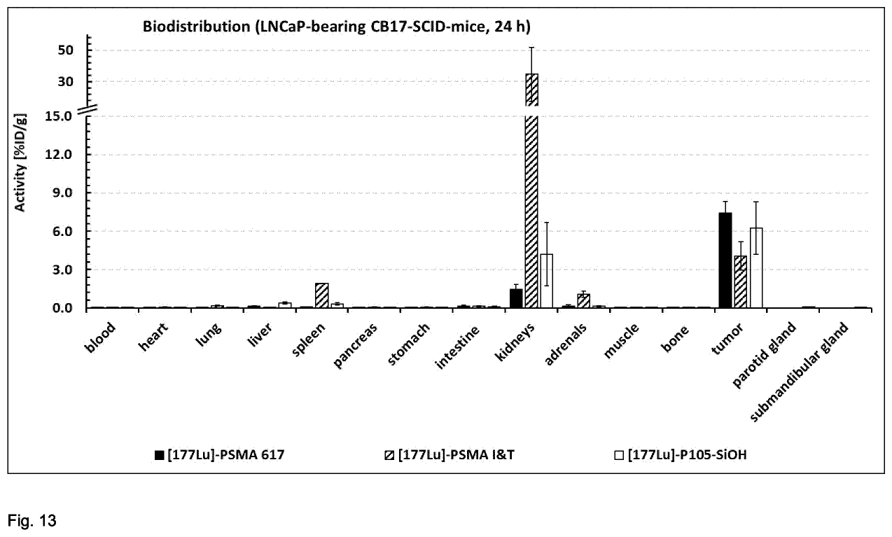

FIG. 13 and the following table show the biodistribution data 24 h p.i. of ¹⁷⁷Lu-labeled compounds in LNCaP tumor-bearing CB17-SCID mice. Data are expressed as mean±SD (n=4-5).

| | [¹⁷⁷Lu] | [¹⁷⁷Lu] | [¹⁷⁷Lu] |
|---|---|---|---|
| organs | PSMA I&T | PSMA-617 | P105-SiOH |
| blood | 0.012 ± 0.01 | 0.006 ± 0.01 | 0.004 ± 0.003 |
| heart | 0.05 ± 0.03 | 0.01 ± 0.01 | 0.01 ± 0.01 |
| lung | 0.16 ± 0.03 | 0.04 ± 0.01 | 0.04 ± 0.01 |
| liver | 0.05 ± 0.01 | 0.12 ± 0.06 | 0.37 ± 0.07 |
| spleen | 1.94 ± 1.01 | 0.08 ± 0.01 | 0.31 ± 0.10 |
| pancreas | 0.05 ± 0.02 | 0.01 ± 0.01 | 0.01 ± 0.01 |
| stomach | 0.05 ± 0.02 | 0.02 ± 0.01 | 0.02 ± 0.01 |
| intestine | 0.12 ± 0.06 | 0.12 ± 0.08 | 0.08 ± 0.05 |
| kidneys | 34.7 ± 17.2 | 1.4 ± 0.4 | 4.2 ± 2.5 |
| adrenals | 1.06 ± 0.24 | 0.13 ± 0.12 | 0.12 ± 0.01 |
| muscle | 0.01 ± 0.01 | 0.01 ± 0.01 | 0.004 ± 0.001 |
| bone | 0.01 ± 0.01 | 0.03 ± 0.01 | 0.02 ± 0.01 |
| tumor | 4.1 ± 1.1 | 7.5 ± 0.9 | 6.3 ± 2.1 |
| parotis | | | 0.07 ± 0.03 |
| submandibularis | | | 0.03 ± 0.01 |

### 2.5.5 Biodistribution: E102-SiOH

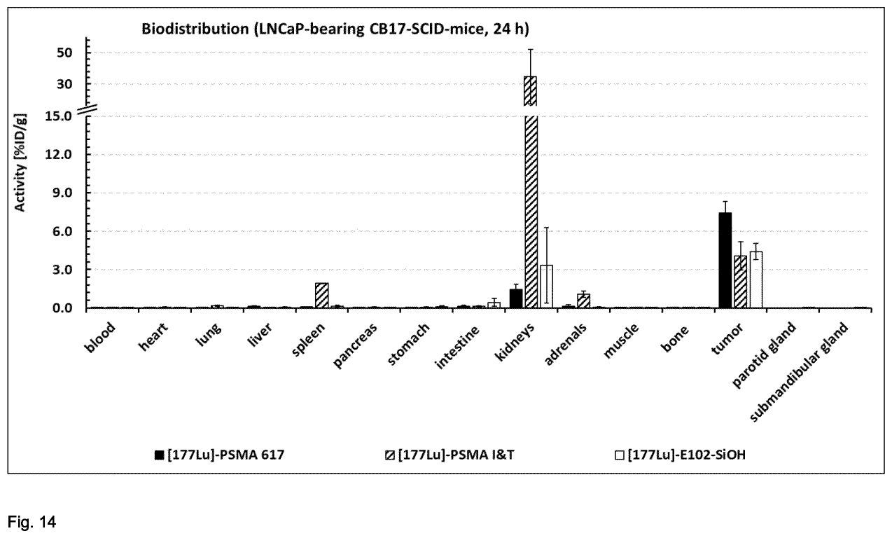

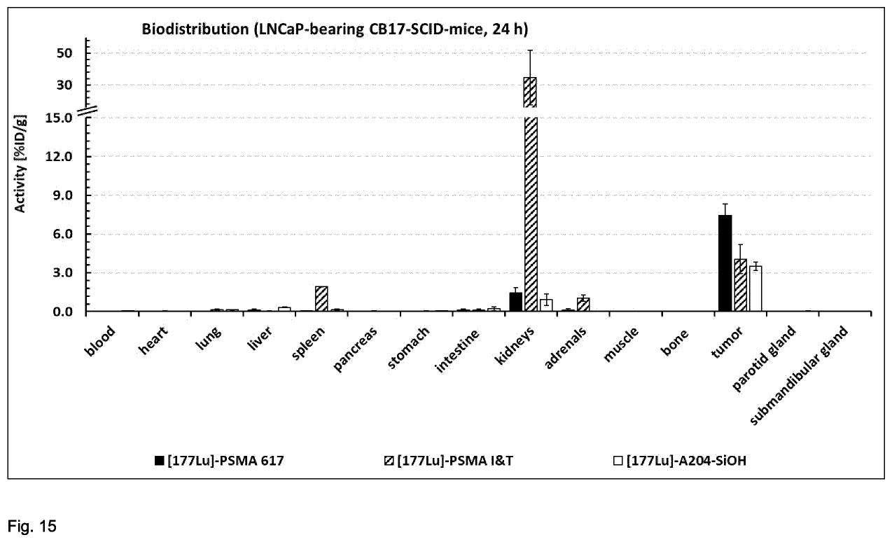

FIG. 14 and the following table show the biodistribution data 24 h p.i. of ¹⁷⁷Lu-labeled compounds in LNCaP tumor-bearing CB17-SCID mice. Data are expressed as mean±SD (n=4-5).

| | [¹⁷⁷Lu] | [¹⁷⁷Lu] | [¹⁷⁷Lu] |
|---|---|---|---|
| organs | PSMA I&T | PSMA-617 | E102-SiOH |
| blood | 0.012 ± 0.01 | 0.006 ± 0.01 | 0.002 ± 0.001 |
| heart | 0.05 ± 0.03 | 0.01 ± 0.01 | 0.02 ± 0.02 |
| lung | 0.16 ± 0.03 | 0.04 ± 0.01 | 0.04 ± 0.02 |
| liver | 0.05 ± 0.01 | 0.12 ± 0.06 | 0.05 ± 0.02 |
| spleen | 1.94 ± 1.01 | 0.08 ± 0.01 | 0.13 ± 0.10 |
| pancreas | 0.05 ± 0.02 | 0.01 ± 0.01 | 0.01 ± 0.01 |
| stomach | 0.05 ± 0.02 | 0.02 ± 0.01 | 0.08 ± 0.07 |
| intestine | 0.12 ± 0.06 | 0.12 ± 0.08 | 0.43 ± 0.31 |
| kidneys | 34.7 ± 17.2 | 1.4 ± 0.4 | 3.3 ± 2.9 |
| adrenals | 1.06 ± 0.24 | 0.13 ± 0.12 | 0.05 ± 0.03 |
| muscle | 0.01 ± 0.01 | 0.01 ± 0.01 | 0.001 ± 0.001 |
| bone | 0.01 ± 0.01 | 0.03 ± 0.01 | 0.03 ± 0.01 |
| tumor | 4.1 ± 1.1 | 7.5 ± 0.9 | 4.4 ± 0.6 |
| parotis | — | — | 0.02 ± 0.01 |
| submandibularis | — | — | 0.01 ± 0.01 |

### 2.5.6 Biodistribution A204-SiOH

FIG. 15 and the following table show the biodistribution data 24 h p.i. of ¹⁷⁷Lu-labeled compounds in LNCaP tumor-bearing CB17-SCID mice. Data are expressed as mean±SD (n=4-5).

| | [¹⁷⁷Lu] | [¹⁷⁷Lu] | [¹⁷⁷Lu] |
|---|---|---|---|
| organs | PSMA I&T | PSMA-617 | A204-SiOH |
| blood | 0.012 ± 0.01 | 0.006 ± 0.01 | 0.061 ± 0.027 |
| heart | 0.05 ± 0.03 | 0.01 ± 0.01 | 0.04 ± 0.01 |
| lung | 0.16 ± 0.03 | 0.04 ± 0.01 | 0.15 ± 0.01 |
| liver | 0.05 ± 0.01 | 0.12 ± 0.06 | 0.34 ± 0.03 |
| spleen | 1.94 ± 1.01 | 0.08 ± 0.01 | 0.15 ± 0.04 |
| pancreas | 0.05 ± 0.02 | 0.01 ± 0.01 | 0.01 ± 0.01 |
| stomach | 0.05 ± 0.02 | 0.02 ± 0.01 | 0.06 ± 0.02 |
| intestine | 0.12 ± 0.06 | 0.12 ± 0.08 | 0.22 ± 0.15 |
| kidneys | 34.7 ± 17.2 | 1.4 ± 0.4 | 0.92 ± 0.45 |
| adrenals | 1.06 ± 0.24 | 0.13 ± 0.12 | 0.0 ± 0.0 |
| muscle | 0.01 ± 0.01 | 0.01 ± 0.01 | 0.008 ± 0.003 |
| bone | 0.01 ± 0.01 | 0.03 ± 0.01 | 0.02 ± 0.01 |
| tumor | 4.1 ± 1.1 | 7.5 ± 0.9 | 3.5 ± 0.3 |
| parotis | | | 0.06 ± 0.01 |
| submandibularis | | | 0.04 ± 0.01 |

### 2.5.7 Discussion of Results

The examined ¹⁷⁷Lu-labelled inhibitor compounds showed the typical uptake pattern of PSMA-addressing ligands in mice 24 h p.i. with high uptake in PSMA-expressing tissues like kidneys and tumor but also in spleen and adrenal glands. The ¹⁷⁷Lu—SiOH-based ligand compounds showed lower accumulation in most of the analysed tissues and blood pool compared to the ¹⁷⁷Lu-rhPSMA compounds These results indicate a substantially decreased HSA binding and confirm the results of the RIAC-based determinations in which SiOH-PSMAs demonstrated significantly lower HSA binding compared to rhPSMAs. The tumor uptakes of the novel SiOH-based ligand compounds were lower compared to ¹⁷⁷Lu-rhPSMA-7.3 and -10 l, which can most probably be attributed to the decreased plasma protein binding and the resulting faster excretion observed for SiOH-PSMAs. Nevertheless, tumor uptakes were still in the same range or higher as determined for the state-of-the-art reference compound ¹⁷⁷Lu-PSMA I&T. Unexpectedly, all SiOH-PSMA ligands showed very low accumulation in kidneys 24 h post injection (<5% ID/g), confirming fast excretion kinetics.

1. A ligand compound, comprising:
(a) a targeting group,
(b) one or more chelating groups, optionally containing a chelated radioactive or non-radioactive cation, and
(c) a group carrying an Si—OH functional moiety.

2. The ligand compound in accordance with claim 1, wherein the group carrying an Si—OH functional moiety is a group of formula (S-1) 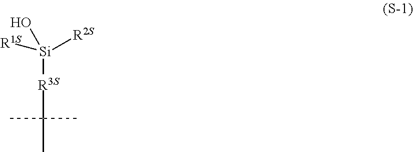
wherein
R^{1S} and R^{2S} are independently a linear or branched C3 to C10 alkyl group;
R^{3S} is a C1 to C20 hydrocarbon group which comprises one or more aromatic and/or aliphatic units, and which optionally comprises up to 3 heteroatoms independently selected from O, N and S;
and wherein the group carrying an Si—OH functional moiety of formula (S-1) is attached to the remainder of the compound via the bond marked by the dashed line.

3. The ligand compound in accordance with claim 1 or 2, wherein the targeting group is a PSMA binding group of formula (P-1) or a pharmaceutically acceptable salt thereof 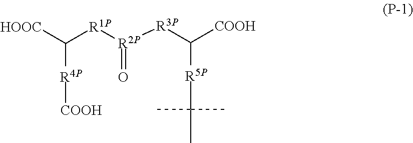
wherein:
R^{1P} is CH₂, NH or O;
R^{3P} is CH₂, NH or O;
R^{2P} is C or P(OH);
R^{4P} is selected from
a group —(CH₂)ₘ—, wherein m is an integer of 2 to 6, and
a group *—(CH₂)ₚ—NH—C(O)—, wherein p is an integer of 1 to 5, and the bond marked with * faces upwards from R^{4P} in formula (P-1);
R^{5P} is selected from
a group —(CH₂)ₙ—, wherein n is an integer of 1 to 6, and
a group *—(CH₂)_{q}—NH—C(O)—, wherein q is an integer of 1 to 5, and the bond marked with * faces upwards from R^{5P} in formula (P-1);
and wherein the PSMA binding group is attached to the remainder of the compound via the bond marked by the dashed line.

4. The ligand compound in accordance with any of claims 1 to 3, wherein the chelating group comprises at least one of
(i) a macrocyclic ring structure with 8 to 20 ring atoms of which 2 or more, preferably 3 or more, are selected from oxygen atoms and nitrogen atoms; and
(ii) an acyclic, open chain chelating structure with 8 to 20 main chain atoms of which 2 or more, preferably 3 or more are heteroatoms selected from oxygen atoms and nitrogen atoms.

5. The ligand compound in accordance with any of claims 1 to 4, wherein the targeting group is a PSMA binding group of formula (P-2) or a pharmaceutically acceptable salt thereof 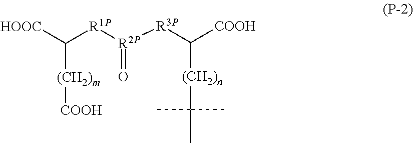
wherein:
m is an integer of 2 to 6, preferably 2 to 4, more preferably 2;
n is an integer of 1 to 6, preferably 2 to 4, more preferably 2 or 4;
R^{1P} is CH₂, NH or O, preferably NH;
R^{3P} is CH₂, NH or O, preferably NH;
R^{2P} is C or P(OH), preferably C;
wherein the PSMA binding group is attached to the remainder of the compound via the bond marked by the dashed line;
wherein the chelating group is selected from a group of the formula (CH-1) or (CH-2), or a pharmaceutically acceptable salt thereof 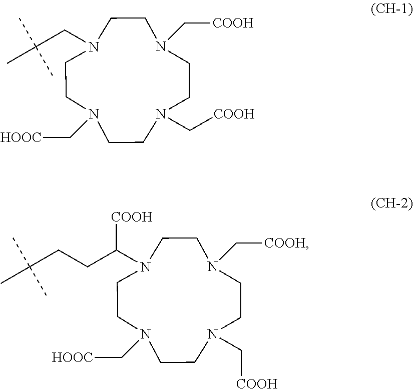
which chelating group is attached by the bond marked by the dashed line to the remainder of the compound via an ester bond or an amide bond, preferably via an amide bond, and wherein the chelating group optionally contains a chelated radioactive or non-radioactive cation, and
wherein the group carrying an Si—OH functional moiety is a group of formula (S-2) or (S-3) 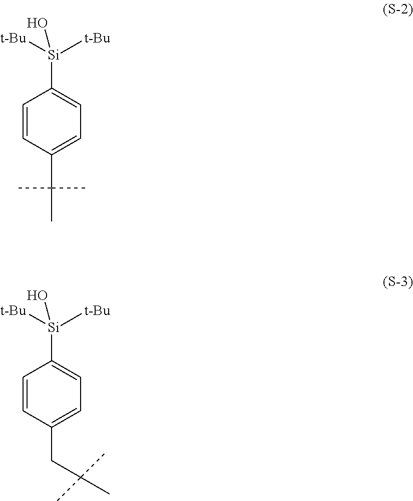
wherein t-Bu indicates a tert-butyl group,
and wherein the group carrying an Si—OH functional moiety of formula (S-2) and (S-3) is attached to the remainder of the compound via the bond marked by the dashed line.

6. The ligand compound in accordance with claim 1, which is a PSMA ligand compound of formula (I) or a pharmaceutically acceptable salt thereof 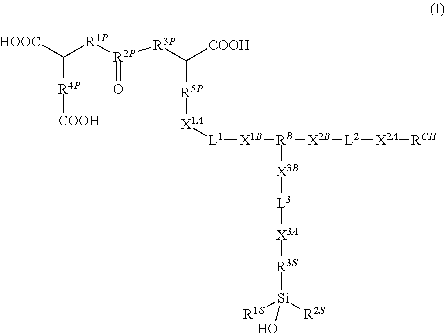
wherein, in formula (I),
R^{1P} is CH₂, NH or O;
R^{3P} is CH₂, NH or O;
R^{2P} is C or P(OH);
R^{4P} is selected from
a group —(CH₂)ₘ—, wherein m is an integer of 2 to 6, and
a group *—(CH₂)ₚ—NH—C(O)—, wherein p is an integer of 1 to 5, and the bond marked with * faces upwards from R^{4P} in formula (I);
R^{5P} is selected from
a group —(CH₂)ₙ—, wherein n is an integer of 1 to 6, and
a group *—(CH₂)_{q}—NH—C(O)—, wherein q is an integer of 1 to 5, and the bond marked with * faces upwards from R^{5P} in formula (I);
X^{1A} is selected from a covalent bond, an amide bond, an ether bond, a thioether bond, an ester bond, a thioester bond, a urea bond, a thiourea bond, and an amine bond;
L¹ is a divalent linking group;
X^{1B} is selected from a covalent bond, an amide bond, an ether bond, a thioether bond, an ester bond, a thioester bond, a urea bond, a thiourea bond, and an amine bond;
R^{B} is a trivalent linking group;
X^{2A} is selected from a covalent bond, an amide bond, an ether bond, a thioether bond, an ester bond, a thioester bond, a urea bond, a thiourea bond, and an amine bond;
L² is a divalent linking group;
X^{2B} is selected from a covalent bond, an amide bond, an ether bond, a thioether bond, an ester bond, a thioester bond, a urea bond, a thiourea bond, and an amine bond;
or —X^{2B}-L² is absent, such that X^{2A} is directly linked to R^{B}
R^{CH} is a chelating group, optionally containing a chelated radioactive or non-radioactive cation;
X^{3A} is selected from a covalent bond, an amide bond, an ether bond, a thioether bond, an ester bond, a thioester bond, a urea bond, a thiourea bond, an amine bond, and a group —NR₂⁺—, wherein the groups R are each an alkyl group, preferably a methyl group;
L³ is a divalent linking group;
X^{3B} is selected from a covalent bond, an amide bond, an ether bond, a thioether bond, an ester bond, a thioester bond, a urea bond and an amine bond;
or —X^{3B}-L³ is absent, such that X^{3A} is directly linked to R^{B};
R^{1S} and R^{2S} are independently a linear or branched C3 to C10 alkyl group; and
R^{3S} is a C1 to C20 hydrocarbon group which comprises one or more aromatic and/or aliphatic units, and which optionally comprises up to 3 heteroatoms selected from O, N and S.

7. The ligand compound in accordance with claim 6, wherein X^{2A} is an ester bond, an amide bond, or a thiourea bond, wherein the chelating group R^{CH} is a residue of a chelating agent selected from bis(carboxymethyl)-1,4,8,11-tetraazabicyclo[6.6.2]-hexadecane (CBTE2a), cyclohexyl-1,2-diaminetetraacetic acid (CDTA), 4-(1,4,8,11-tetraazacyclotetradec-1-yl)-methylbenzoic acid (CPTA), N′-[5-[acetyl(hydroxy)amino]-pentyl]-N-[5-[[4-[5-aminopentyl-(hydroxy)amino]-4-oxobutanoyl]amino]pentyl]-N-hydroxybutandiamide (DFO), 4,11-bis(carboxymethyl)-1,4,8,11-tetraazabicycle-[6.6.2]hexadecan (DO2A) 1,4,7,10-tetraazacyclododecan-N,N′,N″,N″′-tetraacetic acid (DOTA), 2-[1,4,7,10-tetraazacyclododecane-4,7,10-triacetic acid]-pentanedioic acid (DOTAGA), N,N′-dipyridoxylethylendiamine-N,N′-diacetate-5,5′-bis(phosphat) (DPDP), diethylenetriaminepentaacetic acid (DTPA), ethylenediamine-N,N′-tetraacetic acid (EDTA), ethyleneglykol-O,O-bis(2-aminoethyl)-N,N,N′,N′-tetraacetic acid (EGTA), N,N-bis(hydroxybenzyl)-ethylenediamine-N,N′-diacetic acid (HBED), hydroxyethyldiaminetriacetic acid (HEDTA), 1-(p-nitrobenzyl)-1,4,7,10-tetraazacyclo-decan-4,7,10-triacetate (HP-DOA3), 6-hydrazinyl-N-methylpyridine-3-carboxamide (HYNIC), 1,4,7-triazacyclononan-1-succinic acid-4,7-diacetic acid (NODASA), 1-(1-carboxy-3-carboxypropyl)-4,7-(carbooxy)-1,4,7-triazacyclononane (NODAGA), 1,4,7-triazacyclononanetriacetic acid (NOTA), 4,11-bis(carboxymethyl)-1,4,8,11-tetraaza-bicyclo[6.6.2]hexadecane (TE2A), 1,4,8,11-tetraazacyclododecane-1,4,8,11-tetra-acetic acid (TETA), terpyridine-bis(methyleneamine) tetraacetic acid (TMT), 1,4,7,10-tetraazacyclotridecan-N,N′,N″,N″′-tetraacetic acid (TRITA), and triethylenetetra-aminehexaacetic acid (TTHA), N,N′-bis[(6-carboxy-2-pyridil)methyl]-4,13-diaza-18-crown-6 (H₂macropa), 4-amino-4-{2-[(3-hydroxy-1,6-dimethyl-4-oxo-1,4-dihydro-pyridin-2-ylmethyl)-carbamoyl]-ethyl} heptanedioic acid bis-[(3-hydroxy-1,6-dimethyl-4-oxo-1,4-dihydro-pyridin-2-ylmethyl)-amide] (THP), and 1,4,7-triazacyclononane-1,4,7-tris[methylene(2-carboxyethyl)phosphinic acid (TRAP), 2-(4,7,10-tris(2-amino-2-oxoethyl)-1,4,7,10-tetraazacyclododecan-1-yl)acetic acid (DO3AM), 1,4,7,10-tetraazacyclododecane-1,4,7,10-tetrakis[methylene(2-carboxyethylphosphinic acid)](DOTPI), and S-2-(4-isothiocyanatobenzyl)-1,4,7,10-tetraazacyclododecane tetraacetic acid,
and the bond X^{2A} is formed using a functional group contained in the chelating agent, and wherein the chelating group R^{CH} optionally contains a chelated radioactive or non-radioactive cation.

8. The ligand compound in accordance with claim 6 or 7, wherein the chelating group contains a chelated cation selected from the cations of ⁴³Sc, ⁴⁴Sc, ⁴⁷Sc, ⁵¹Cr, ^{52m}Mn, ⁵⁸Co, ⁵²Fe, ⁵⁶Ni, ⁵⁷Ni, ⁶²Cu, ⁶⁴Cu, ⁶⁷Cu, ⁶⁶Ga, ⁶⁸Ga, ⁶⁷Ga, ⁸⁹Zr, ⁹⁰Y, ⁸⁶Y, ⁹⁴mTc, ⁹⁹mTc, ⁹⁷Ru, ¹⁰⁵Rh, ¹⁰⁹Pd, ¹¹¹Ag, ^{110m}In, ¹¹¹In, ^{113m}In, ^{114m}In, ^{117m}Sn, ¹²¹Sn, ¹²⁷Te, ¹⁴²Pr, ¹⁴³Pr, ¹⁴⁷Nd, ¹⁴⁹Gd, ¹⁴⁹Pm, ¹⁵¹Pm, ¹⁴⁹Tb, ¹⁵²Tb, ¹⁵⁵Tb, ¹⁵³Sm, ¹⁵⁶Eu, ¹⁵⁷Gd, ¹⁶¹Tb, ¹⁶⁴Tb, ¹⁶¹Ho, ¹⁶⁶Ho, ¹⁵⁷Dy, ¹⁶⁵Dy, ¹⁶⁶Dy, ¹⁶⁰Er, ¹⁶⁵Er, ¹⁶⁹Er, ¹⁷¹Er, ¹⁶⁶Yb, ¹⁶⁹Yb, ¹⁷⁵Yb, ¹⁶⁷Tm, ¹⁷²Tm, ^{nat}Lu, ¹⁷⁷Lu, ¹⁸⁶Re, ¹⁸⁸Re, ¹⁸⁸W, ¹⁹¹Pt, ^{195m}Pt, ¹⁹⁴Ir, ¹⁹⁷Hg, ¹⁹⁸Au, ¹⁹⁹Au, ²¹²Pb, ²⁰³Pb, ²¹¹At, ²¹²Bi, ²¹³Bi, ²²³Ra, ²²⁴Ra, ²²⁵Ac, and ²²⁷Th, or a cationic molecule comprising ¹⁸F, such as ¹⁸F-[AIF]²⁺.

9. The ligand compound in accordance with any of claims 6 to 8, wherein L¹ comprises two or more subunits which form a chain of subunits between X^{1A} and X^{1B},
wherein the bond(s) between the subunits in the chain of subunits is (are) independently selected for each occurrence from an amide bond, an ether bond, a thioether bond, an ester bond, a thioester bond, a urea bond, a thiourea bond and an amine bond.

10. The ligand compound in accordance with any of claims 6 to 9,
wherein —X^{2B}-L² is absent, or wherein the group L² is an alkanediyl group, preferably a linear alkanediyl group,
which may be substituted by one or more substituents independently selected from —OH, —OCH₃, —COOH, —COOCH₃, —NH₂, —CONH₂, —NHC(O)NH₂, and —NHC(NH)NH₂;
and wherein one or more covalent bonds between carbon atoms in the alkanediyl group may be independently replaced by a bond selected from an amide bond, an ether bond, a thioether bond, an ester bond, a thioester bond, a urea bond, a thiourea bond, and an amine bond.

11. The ligand compound in accordance with any of claims 6 to 10, wherein —X^{3B}-L³ is absent, or wherein the group L³ is an alkanediyl group, preferably a linear alkanediyl group,
which may be substituted by one or more substituents independently selected from —OH, —OCH₃, —COOH, —COOCH₃, —NH₂, —CONH₂, —NHC(O)NH₂, and —NHC(NH)NH₂;
and wherein one or more covalent bonds between carbon atoms in the alkanediyl group may be independently replaced by a bond selected from an amide bond, an ether bond, a thioether bond, an ester bond, a thioester bond, a urea bond, a thiourea bond, and an amine bond.

12. The ligand compound in accordance with any of claims 6 to 11, wherein R^{B} in formula (I) is a group of the formula (B-1): 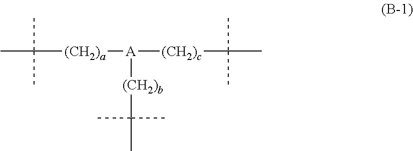
wherein
A is selected from N, CR^{B1} wherein R^{B1} is H or C₁-C₆ alkyl, and a 5 to 7 membered carbocyclic or heterocyclic group;
the bond marked by the dashed line at (CH₂)ₐ is formed with X^{1B}, and a is an integer of 0 to 4;
the bond marked by the dashed line at (CH₂)_{b} is formed with X^{3B}, if present, and otherwise with X^{3A}, and b is an integer of 0 to 4;
and the bond marked by the dashed line at (CH₂)_{c} is formed with X^{2B}, if present, and otherwise with X^{2A}, and c is an integer of 0 to 4.

13. The ligand compound in accordance with any of claims 6 to 12, which is a compound of formula (II) or a pharmaceutically acceptable salt thereof 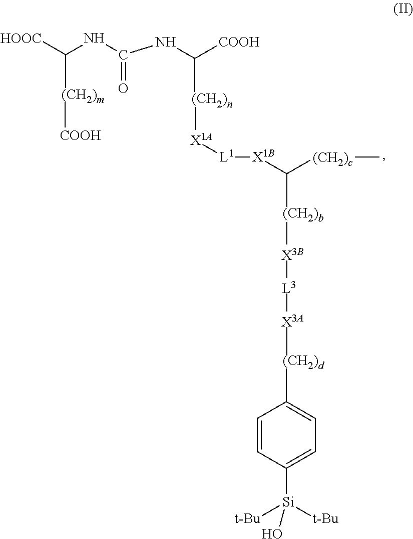
wherein, in formula (II), m, n, b, c, X^{1A}, L¹, X^{1B}, X^{2B}, L², X^{2A}, R^{CH}, X^{3A}, L³ and X^{3B} are defined as in the preceding claims 6 to 12, and d is 0 or 1.

14. The ligand compound in accordance with any of claims 6 to 13, wherein —X^{2A}—R^{CH} is a group of the formula (XCH-1) or (XCH-2) 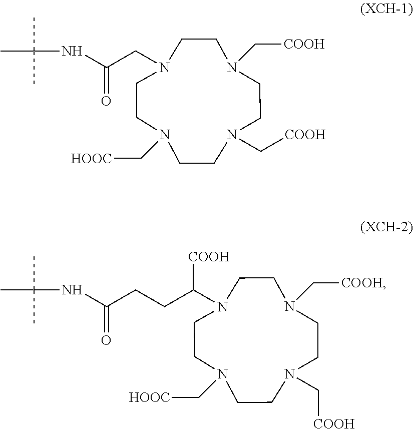
which is attached to the remainder of the compound via the bond marked by the dashed line, and wherein the chelating group R^{CH} optionally contains a chelated radioactive or non-radioactive cation.

15. The ligand compound in accordance with any of claims 6 to 14, wherein the group —X^{1A}-L¹-X^{1B}— in formula (I) is a group of any of the formulae (L-1) to (L-6):
*—NH—C(O)—R^{1L}—C(O)—NH—R^{2L}—NH—C(O)— (L-1)
*—C(O)—NH—R^{3L}—NH—C(O)—R^{4L}—C(O)—NH—R^{5L}—NH—C(O)— (L-2)
*—C(O)—NH—R^{6L}—NH—C(O)—R^{7L}—NH—C(O)—R^{8L}—NH—C(O)—R^{9L}—NH—C(O)— (L-3)
*—C(O)—NH—R^{10L}—NH—C(O)—R^{11L}—NH—C(O)— (L-4)
*—C(O)—NH—R^{12L}—NH—C(O)—R^{13L}—C(O)—NH—R^{14L}—NH—C(O)—R^{15L}—NH—C(O)— (L-5)
*—C(O)—NH—R^{16L}—C(O)—NH—R^{17L}—C(O)—NH—R^{18L}—C(O)—NH— (L-6)
wherein each of R^{1L} to R^{18L} is independently an alkanediyl group containing 1 to 8 carbon atoms, preferably a linear alkanediyl group containing 1 to 8 carbon atoms,
wherein each of R^{1L} to R^{18L} may be substituted by one or more substitutents independently selected from —OH, —OCH₃, —COOH, —COOCH₃, —NH₂, —CONH₂, —NHC(O)NH₂, and —NHC(NH)NH₂;
and wherein * marks the bond corresponding to the X^{1A} terminal bond of —X^{1A}-L¹-X^{1B}—.

16. The ligand compound in accordance with any of claims 6 to 14, wherein the group —X^{1A}-L¹-X^{1B}— in formula (I) is a group of the formula (L-7):
—NH—C(O)—R^{19L}—NH—C(O)—R^{20L}—NH—C(O)—R^{21L}—NH—C(O)— (L-7)
wherein R^{19L} is an alkanediyl group containing 1 to 8 carbon atoms, preferably a linear alkanediyl group containing 1 to 8 carbon atoms;
R^{20L} is an alkanediyl group containing 1 to 8 carbon atoms, a cycloalkanediyl group containing 3 to 6 carbon atoms or an alkanediyl-cycloalkanediyl group containing 5 to 8 carbon atoms,
wherein each of R^{19L} an R^{20L} may be substituted by one or more substituents independently selected from —OH, —OCH₃, —COOH, —COOCH₃, —NH₂, —CONH₂, —NHC(O)NH₂, —NHC(NH)NH₂, aryl and aralkyl;
R^{21L} is an alkanediyl group containing 2 to 26 carbon atoms, preferably a linear alkanediyl group containing 2 to 26 carbon atoms, wherein one or more —CH₂— groups may be replaced by —O—;
and wherein * marks the bond corresponding to the X^{1A} terminal bond of —X^{1A}-L¹-X^{1B}—.

17. A ligand compound in accordance with any one of claims 1 to 16 for use in a therapeutic or diagnostic method.

18. A ligand compound in accordance with any one of claims 1 to 16 for use in a method of treating or diagnosing cancer.

19. A ligand compound for use in accordance with claim 18, wherein the cancer is prostate cancer.
